SEQUENCE LISTING (24 sequences)

SEQ ID NO: 1 - DNA, 16 bp, Artificial Sequence
  Description of Artifical Sequence Synthetic oligonucleotide
ctcaatccatggcagc

SEQ ID NO: 2 - DNA, 21 bp, Artificial Sequence
  Description of Combined DNA/RNA Molecule Synthetic oligonucleotide
gcaugcggccucuguuugatt

SEQ ID NO: 3 - DNA, 21 bp, Artificial Sequence
  Description of Combined DNA/RNA Molecule Synthetic oligonucleotide
ucaaacagaggccgcaugctt

SEQ ID NO: 4 - DNA, 19 bp, Artificial Sequence
  Description of Artifical Sequence Synthetic oligonucleotide
tctcccagcgtgcgcccat

SEQ ID NO: 5 - DNA, 16 bp, Artificial Sequence
  Description of Artifical Sequence Synthetic oligonucleotide
tggcaagcatcctgta

SEQ ID NO: 6 - DNA, 16 bp, Artificial Sequence
  Description of Artifical Sequence Synthetic oligonucleotide
tagcctgtcacttctc

SEQ ID NO: 7 - DNA, 16 bp, Artificial Sequence
  Description of Artifical Sequence Synthetic oligonucleotide
gctccagacatcactc

SEQ ID NO: 8 - DNA, 16 bp, Artificial Sequence
  Description of Artifical Sequence Synthetic oligonucleotide
agccattcattccacc

SEQ ID NO: 9 - DNA, 16 bp, Artificial Sequence
  Description of Artifical Sequence Synthetic oligonucleotide
ttattgtgcatctcag

SEQ ID NO: 10 - DNA, 16 bp, Artificial Sequence
  Description of Artifical Sequence Synthetic oligonucleotide
cgtgtatttccgcgtg

SEQ ID NO: 11 - DNA, 16 bp, Artificial Sequence
  Description of Artifical Sequence Synthetic oligonucleotide
ggcacagccagtggcg

SEQ ID NO: 12 - DNA, 16 bp, Artificial Sequence
  Description of Artifical Sequence Synthetic oligonucleotide
cccaaggcactgcaga

SEQ ID NO: 13 - DNA, 16 bp, Artificial Sequence
  Description of Artifical Sequence Synthetic oligonucleotide
accaagtttcttcagc

SEQ ID NO: 14 - DNA, 16 bp, Artificial Sequence
  Description of Artifical Sequence Synthetic oligonucleotide
ctccttggtgcagtct

SEQ ID NO: 15 - DNA, 15 bp, Artificial Sequence
  Description of Artifical Sequence Synthetic oligonucleotide
tcagattcaaaccca

SEQ ID NO: 16 - DNA, 16 bp, Artificial sequence
  Description of Artifical Sequence Synthetic oligonucleotide
gtgttctacaccatta

SEQ ID NO: 17 - protein, 12 aa, Artificial sequence
  Description of Artifical Sequence Synthetic peptide
CYGRKKRRQRRR

SEQ ID NO: 18 - protein, 10 aa, Artificial sequence
  Description of Artifical Sequence Synthetic peptide
CRRRRRRRRR

SEQ ID NO: 19 - protein, 23 aa, Artificial sequence
  Description of Artifical Sequence Synthetic peptide
CYGRKKRRQRRRYGRKKRRQRRR

SEQ ID NO: 20 - protein, 4 aa, Artificial sequence
  Description of Artifical Sequence Synthetic peptide
RGDC

SEQ ID NO: 21 - protein, 5 aa, Artificial sequence
  Description of Artifical Sequence Synthetic peptide
RGDFC

SEQ ID NO: 22 - protein, 5 aa, Artificial sequence
  Description of Artifical Sequence Synthetic peptide
RGDFK

SEQ ID NO: 23 - protein, 19 aa, Artificial sequence
  Description of Artifical Sequence Synthetic peptide
CYGRKKRRQRRRGGGRGDS

SEQ ID NO: 24 - protein, 9 aa, Artificial sequence
  Description of Artifical Sequence Synthetic peptide
RRRRRRRRR

(19) United States
(12) Patent Application Publication
(10) Pub. No.: US 2011/0223257 A1
(43) Pub. Date: Sep. 15, 2011

(54) RELEASABLE FUSOGENIC LIPIDS FOR NUCLEIC ACIDS DELIVERY SYSTEMS

(75) Inventors: Hong Zhao, Edison, NJ (US); Weili Yan, Piscataway, NJ (US); Lianjun Shi, Bridgewater, NJ (US); Dechun Wu, Bridgewater, NJ (US); Maksim Royzen, Brooklyn, NY (US)

(73) Assignee: ENZON PHARMACEUTICALS, INC., Bridgewater, NJ (US)

(21) Appl. No.: 13/129,546

(22) PCT Filed: Nov. 17, 2009
(86) PCT No.: PCT/US09/64730

§ 371 Date: May 16, 2011

Related U.S. Application Data
(60) Provisional application No. 61/115,378, filed on Nov. 17, 2008.

(51) Int. Cl. A61K 9/50; C07C 229/34; C07C 233/87; C07F 9/02; C12N 5/00; A61K 47/00; A61K 31/7088; A61K 31/56; B82Y 5/00

(52) U.S. Cl. 424/502; 554/107; 554/106; 554/36; 554/84; 435/375; 514/785; 514/44 R; 514/182; 977/773

(57) ABSTRACT

The present invention relates to releasable fusogenic lipids and nanoparticle compositions containing the same for the delivery of oligonucleotides and methods of modulating gene expression using the same. In particular, this invention relates to releasable fusogenic lipids containing an imine linker and a zwitterionic moiety.

### CROSS-REFERENCE TO RELATED APPLICATION

This application claims the benefit of priority from U.S. Provisional Patent Application Ser. No. 61/115,378, filed Nov. 17, 2008, the contents of which are incorporated herein by reference.

### BACKGROUND OF THE INVENTION

Therapy using nucleic acids has been proposed as an endeavor to treat various diseases over the past years. Therapy such as antisense therapy is a powerful tool in the treatment of disease because a therapeutic gene can selectively modulate gene expression associated with disease and minimize side effects which occur when other therapeutic approaches are used.

Therapy using nucleic acids has, however, been limited due to poor stability of genes and ineffective delivery. Several gene delivery systems have been proposed to overcome the hurdles and effectively introduce therapeutic genes into the target area, such as cancer cells or tissues in vitro and in vivo. Such attempts to improve delivery and enhance cellular uptake of therapeutic genes are directed to utilizing liposomes.

Currently available liposomes do not effectively deliver oligonucleotides into the body, although some progress has been made in the delivery of plasmids. In the delivery of oligonucleotides, desirable delivery systems should include positive charges sufficient enough to neutralize the negative charges of oligonucleotides. Recently, coated cationic liposomal (CCL) and Stable Nucleic Acid-Lipid Particles (SNALP) formulations described by Stuart, D. D., et al Biochim. Biophys. Acta, 2000, 1463:219-229 and Semple, S. C., et al, Biochim. Biophys. Acta, 2001, 1510:152-166, respectively, were reported to provide nanoparticles with small sizes, high nucleic acid encapsulation rate, good serum stability, and long circulation time.

In spite of the attempts and advances, there continues to be a need to provide improved nucleic acids delivery systems. The present invention addresses this need.

### SUMMARY OF THE INVENTION

The present invention provides releasable fusogenic lipids containing an imine linker and a zwitterionic moiety, and nanoparticle compositions containing the same for nucleic acids delivery. Polynucleic acids, such as oligonucleotides, are encapsulated within nanoparticle complexes containing a mixture of a cationic lipid, a releasable fusogenic lipid described herein, and a PEG lipid.

In accordance with this aspect of the invention, the releasable fusogenic lipids for the delivery of nucleic acids (i.e., an oligonucleotide) have Formula (I):

R-(L₁)ₐ-M-(L₂)_{b}-Q

wherein

R is a water soluble neutral charged or zwitterion-containing moiety;

L₁₋₂ are independently selected bifunctional linkers;

M is an imine-containing moiety;

Q is a substituted or unsubstituted, saturated or unsaturated C4-30-containing moiety;

(a) is 0 or a positive integer; and

(b) is 0 or a positive integer.

The present invention also provides nanoparticle compositions for nucleic acids delivery. According to the present invention, the nanoparticle composition for the delivery of nucleic acids (i.e., an oligonucleotide) can include:

(i) a cationic lipid;

(ii) a compound of Formula (I); and

(iii) a PEG lipid.

In another aspect of the present invention, there are provided methods of delivering nucleic acids (preferably oligonucleotides) to a cell or tissue, in vivo and in vitro. Oligonucleotides introduced by the methods described herein can modulate expression of a target gene.

Another aspect of the present invention provides methods of inhibiting expression of a target gene, i.e., oncogenes and genes associated with disease in mammals, preferably humans. The methods include contacting cells, such as cancer cells or tissues, with a nanoparticle/nanoparticle complex prepared from the nanoparticle composition described herein. The oligonucleotides encapsulated within the nanoparticle are released, which then mediate the down-regulation of mRNA or protein in the cells or tissues being treated. The treatment with the nanoparticle allows modulation of target gene expression (and the attendant benefits associated therewith) in the treatment of malignant disease, such as inhibition of the growth of cancer cells. Such therapies can be carried out as a single treatment or as part of a combination therapy, with one or more useful and/or approved treatments.

Further aspects include methods of making the compounds of Formula (I) as well as nanoparticles containing the same.

The nanoparticle composition containing a releasable fusogenic lipid described herein provides a means for in vivo as well as in vitro administration of nucleic acids.

The nanoparticles containing the releasable fusogenic lipids described herein can help release nucleic acids encapsulated therein when the nanoparticles enter the cells and cellular compartments. Without being bound by any theory, such feature is attributed in part to the acid labile linker. The imine-based linkers are acid-labile and hydrolyzed in acidic environment such is as cancer cells and endosome. Thus, the imine-based linkers can facilitate disruption of the nanoparticles, thereby allowing intracellular release of nucleic acids.

The releasable fusogenic lipids containing zwitterionic charged groups enhance cellular uptake of nucleic acids. The polar but neutrally charged groups facilitate the nanoparticles to cross the cellular membrane.

The releasable fusogenic lipids described herein stabilize nanoparticle complexes and nucleic acids therein in biological fluids. The nanoparticle complexes can shield nucleic acids molecules from nucleases, thereby protecting the polynucleic acids from degradation.

The nanoparticle delivery systems described herein allow sufficient amounts of the therapeutic oligonucleotides to be selectively available at the desired target area, such as cancer cells via EPR (Enhanced Permeation and Retention) effects. The therapeutic nucleic acids at the target area can modulate expression of a target gene specifically in cancer cells or tissues.

The nanoparticles described herein can also be used in the delivery of biologically active molecules, such as small molecule chemotherapeutics as well as one or more different types of therapeutic nucleic acids, thereby attaining synergistic effects in the treatment of disease.

Other and further advantages will be apparent from the following description.

For purposes of the present invention, the term “residue” shall be understood to mean that portion of a compound, to which it refers, e.g., C6-30 hydrocarbons, etc. that remains after it has undergone a substitution reaction with another compound.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) preferably include halo, oxy, azido, nitro, cyano, alkyl, alkoxy, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) preferably include halo, oxy, azido, nitro, cyano, alkyl, alkoxy, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl. For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated number of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkloxy group.

For purposes of the present invention, the term “alkyl-thio-alkyl” refers to an alkyl-S-alkyl thioether, for example methylthiomethyl or methylthioethyl.

For purposes of the present invention, the term “amino” refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “alkylcarbonyl” refers to a carbonyl group substituted with alkyl group.

For purposes of the present invention, the term “halogen' or “halo” refers to fluorine, chlorine, bromine, and iodine.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one hetero atom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

For purposes of the present invention, the term “heteroatom” refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalryls include moieties such as 3-methoxythiophene; alkoxy includes moieties such as methoxy; and phenoxy includes moieties such as 3-nitrophenoxy. Halo shall be understood to include fluoro, chloro, iodo and bromo.

For purposes of the present invention, “positive integer” shall be understood to include an integer equal to or greater than I and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art. The term “nanoparticle” and/or “nanoparticle complex” formed using the nanoparticle composition described herein refers to a lipid-based nanocomplex. The nanoparticle contains nucleic acids such as oligonucleotides encapsulated in a mixture of a cationic lipid, a fusogenic lipid, and a PEG lipid. Alternatively, the nanoparticle can be formed without nucleic acids.

For purposes of the present invention, the term “therapeutic oligonucleotide” refers to an oligonucleotide used as a pharmaceutical or diagnostic agent.

For purposes of the present invention, “modulation of gene expression” shall be understood as broadly including down-regulation or up-regulation of any types of genes, preferably associated with cancer and inflammation, compared to a gene expression observed in the absence of the treatment with the nanoparticle described herein, regardless of the route of administration.

For purposes of the present invention, “inhibition of expression of a target gene” shall be understood to mean that mRNA expression or the amount of protein translated are reduced or attenuated when compared to that observed in the absence of the treatment with the nanoparticle described herein. Suitable assays of such inhibition include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art. The treated conditions can be confirmed by, for example, decrease in mRNA levels in cells, preferably cancer cells or tissues.

Broadly speaking, successful inhibition or treatment shall be deemed to occur when the desired response is obtained. For example, successful inhibition or treatment can be defined by obtaining e.g, 10% or higher (i.e. 20% 30%, 40%) down regulation of genes associated with tumor growth inhibition. Alternatively, successful treatment can be defined by obtaining at least 20% or preferably 30%, more preferably 40% or higher (i.e., 50% or 80%) decrease in oncogene mRNA levels in cancer cells or tissues, including other clinical markers contemplated by the artisan in the field, when compared to that observed in the absence of the treatment with the nanoparticle described herein.

Further, the use of singular terms for convenience in description is in no way intended to be so limiting. Thus, for example, reference to a composition comprising an oligonucleotide, a cholesterol analog, a cationic lipid, a releasable fusogenic lipid, a PEG lipid etc. refers to one or more molecules of that oligonucleotide, cholesterol analog, cationic lipid, releasable fuosogenic lipid, PEG lipid, etc. It is also contemplated that the oligonucleotide can be the same or different kind of gene. It is also to be understood that this invention is not limited to the particular configurations, process steps, and materials disclosed herein as such configurations, process steps, and materials may vary somewhat.

It is also to be understood that the terminology employed herein is used for the purpose of describing particular embodiments only and is not intended to be limiting, since the scope of the present invention will be limited by the appended claims and equivalents thereof.

### BRIEF DESCRIPTION OF DRAWINGS

FIG. 1 schematically illustrates a reaction scheme for preparing compound 6, as described in Examples 6-11.

FIG. 2 schematically illustrates a reaction scheme for preparing compound 10, as described in Examples 12-15.

### DETAILED DESCRIPTION OF THE INVENTION

### A. Overview

### 1. Releasable Fusogenic Lipids of Formula (I)

In one aspect of the present invention, there are provided compounds of Formula (I):

R-(L₁)ₐ-M-(L₂)_{b}-Q (I)

wherein

R is a water soluble neutral charged or zwitterion-containing moiety;

L₁₋₂ are independently selected bifunctional linkers;

M is an imine-containing moiety;

Q is a substituted or unsubstituted, saturated or unsaturated C4-30-containing moiety;

(a) is 0 or a positive integer, preferably zero or an integer of from about 1 to about 10 (e.g., 1, 2, 3, 4, 5, 6); and

(b) is 0 or a positive integer, preferably zero or an integer of from about 1 to about 10 (e.g., 1, 2, 3, 4, 5, 6).

L₁ and L₂ are independently the same or different when (a) and (b) are equal to or greater than 2.

In one preferred aspect, the compounds of Formula described herein include the Q hydrocarbon group (aliphatic). The Q group has Formula (Ia):

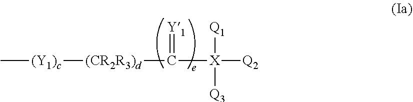

wherein

Y₁ and Y′₁ are independently O, S or NR₄, preferably oxygen;

(c) is 0 or 1;

(d) is 0 or a positive integer, preferably zero or an integer of from about 1 to about 10 (e.g., 1, 2, 3, 4, 5, 6);

(e) is 0 or 1;

X is C, N or P;

Q₁ is H, C₁₋₃ alkyl, NR₅, OH, or

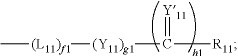

Q₂ is H, C₁₋₃ alkyl, NR₆, OH, or

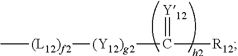

Q₃ is a lone electron pair, (═O), H, C₁₋₃ alkyl, NR₇, OH, or

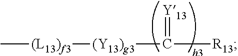

provided that
(i) when X is C, Q₃ is not a lone electron pair or (═O);
(ii) when X is N, Q₃ is a lone electron pair; and
(iii) when X is P, Q₃ is (═O) and (e) is zero,
   wherein
   L₁₁, L₁₂ and L₁₃ are independently selected bifunctional spacers;
   Y₁₁, Y₁₂, and Y₁₃ are independently O, S or NR₈, preferably O or NR₈;
   Y′₁₁, Y′₁₂, and Y′₁₃ are independently O, S or NR₈, preferably oxygen;
   R₁₁, R₁₂ and R₁₃ are independently substituted or unsubstituted, saturated or unsaturated C₄₋₃₀;
   (f1), (f2) and (f3) are independently 0 or 1;
   (g1), (g2) and (g3) are independently 0 or 1; and
   (h1), (h2) and (h3) are independently 0 or 1;

R₂₋₃ are independently selected from the group consisting of hydrogen, hydroxyl, amine, substituted amine, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl, preferably, hydrogen, hydroxyl, amine, methyl, ethyl and propyl; and

R₄₋₈ are independently selected from the group consisting of hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆ heteroalkyl, preferably, hydrogen, methyl, ethyl and propyl,

provided that Q includes at least one or two (e.g., one, two, three) of R₁₁, R₁₂ and R₁₃.

The combinations of the bifunctional linkers and the bifunctional spacers contemplated within the scope of the present invention include those in which combinations of variables and substituents of the linker and spacer groups are permissible so that such combinations result in stable compounds of Formula (I). For example, the combinations of values and substituents do not permit oxygen, nitrogen or carbonyl to be positioned directly adjacent to imine.

Preferably, Q includes at least two of R₁₁, R₁₂ and R₁₃.

The —C(R₂R₃)— group, in each occurrence is the same or different when (d) is equal to or greater than 2.

In one preferred aspect of the invention, the imine-containing moiety has the formula:

—N═CR₁— or —CR₁═N—,

wherein R₁ is hydrogen, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl, preferably, hydrogen, methyl, ethyl, or propyl.

In one embodiment, the acid-labile M linker is —N═CH— or —CH═N—.

According to the present invention, the releasable fusogenic lipids described herein have Formula (Ib) or (I′b):

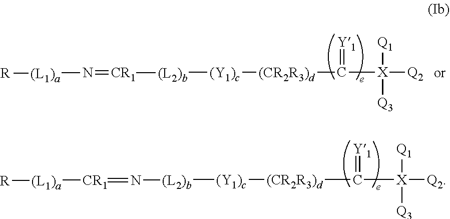

### 2. Water Soluble Neutral Charged or Zwitterion-Containing Moiety: R Group

The compounds described herein include a terminal zwitterion. In one embodiment, the zwitterion includes an amine and an acid. The acidic proton is positioned three to eight atoms from the amine (e.g., the acidic proton is positioned 3, 4, 5, 6, 7, or 8 atoms from the amine). Preferably, the acidic proton is positioned three to six atoms from the amine.

The acid includes, but is not limited to, a carboxylic acid, a sulfonic acid, or a phosphoric acid.

In a further embodiment, the zwitterion-containing moiety is a zwitterionic form of an amino acid. Some illustrative examples of R group include, but are not limited to:

—CH(COO)(NH₃,

Lys=—HN—(CH₂)₄CH(COO)(NH₃),

Glu=—C(=O)—(CH₂)₂CH(COO)(NH₃) and

Asp=—C(═O)—(CH₂)CH(COO)(NH₃).

In another embodiment, the zwitterion-containing moiety is a derivative of zwitterionic form of an amino acid. The amino acid can be naturally-occurring amino acids or derivatives of the naturally occurring amino acids. Some examples amino acid analogs and derivates include: 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethyl glycine, N-ethylasparagine, 3-hydroxyproline, 4-hydroxyproline, isodesmosine, allo-isoleucine, N-methylglycine or sarcosine, N-methyl-isoleucine, 6-N-methyllysine, N-methylvaline, norvaline, norleucine, ornithine, and others too numerous to mention, that are listed in 63 Fed. Reg., 29620, 29622, incorporated by reference herein.

### 3. The Bifunctional Linker: L₁ and L₂ Groups

According to the present invention, the L₁ group as included in the compounds of Formula (I) is selected from among:

—(CR₂₁R₂₂)ₜ₁—[C(═Y₁₆)]ₐ₃—,

—(CR₂₁R₂₂)ₜ₁Y₁₇—(CR₂₃R₂₄)ₜ₂—(Y₁₈)ₐ₂—[C(═Y₁₆)]ₐ₃—,

—(CR₂₁R₂₂CR₂₃R₂₄Y₁₇)ₜ₁—[C(═Y₁₆)]ₐ₃—,

—(CR₂₁R₂₂CR₂₃R₂₄Y₁₇)ₜ₁(CR₂₅R₂₆)ₜ₄—(Y₁₈)ₐ₂—[C(═Y₁₆)]ₐ₃—,

—[(CR₂₁R₂₂CR₂₃R₂₄)ₜ₂Y₁₇]ₜ₃(CR₂₅R₂₆)ₜ₄—(Y₁₈)ₐ₂—[C(═Y₁₆)]ₐ₃—,

—(CR₂₁R₂₂)ₜ₁—[(CR₂₃R₂₄)₁₂Y₁₇](CR₂₅R₂₆)ₜ₄—(Y₁₈)ₐ₂—[C(═Y₁₆)]ₐ₃—,

—(CR₂₁R₂₂)ₜ₁(Y₁₇)ₐ₂[C(═Y₁₆)]ₐ₃(CR₂₃R₂₄)ₜ₂—,

—(CR₂₁R₂₂)ₜ₁(Y₁₇)ₐ₂[C(═Y₁₆)]ₐ₃Y₁₄(CR₂₃R₂₄)ₜ₂—,

—(CR₂₁R₂₂)ₜ₁(Y₁₇)ₐ₂[C(═Y₁₆)]ₐ₃(CR₂₃R₂₄)ₜ₂—Y₁₅—(CR₂₃R₂₄)ₜ₃—,

—(CR₂₁R₂₂)ₜ₁(Y₁₇)ₐ₂[C(═Y₁₆)]ₐ₃Y₁₄(CR₂₃R₂₄)ₜ₂—Y₁₅—(CR₂₃ R₂₄)ₜ₃—,

—(CR₂₁R₂₂)ₜ₁(Y₁₇)ₐ₂[C(═Y₁₆)]ₐ₃(CR₂₃R₂₄CR₂₅R₂₆Y₁₉)ₜ₂(CR₂₇CR₂₈)ₜ₃—,

—(CR₂₁R₂₂)ₜ₁(Y₁₇)ₐ₂[C(═Y₁₆)]ₐ₃Y₁₄(CR₂₃R₂₄CR₂₅R₂₆Y₁₉)ₜ₂(CR₂₇CR₂₈)ₜ₃—, and

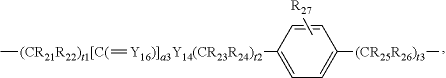

wherein:

Y₁₆ is O, NR₂₈, or S, preferably oxygen;

Y₁₄₋₁₅ and Y₁₇₋₁₉ are independently O, NR₂₉, or S, preferably O, or NR₂₉;

R₂₁₋₂₇ are independently selected from among hydrogen, hydroxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl; and

R₂₈₋₂₉ are independently selected from among hydrogen, C₁₋₆ alkyls, C₃₋₁₂ branched alkyls, C₃₋₈ cycloalkyls, C₁₋₆ substituted alkyls, C₃₋₈, substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl;

(t1), (t2), (t3) and (t4) are independently zero or positive integers, preferably zero or a positive integer of from about 1 to about 10 (e.g., 1, 2, 3, 4, 5, 6); and

(a2) and (a3) are independently zero or 1.

The bifunctional L₁ linkers contemplated within the scope of the present invention include those in which combinations of substituents and variables are permissible so that such combinations result in stable compounds of Formula (I). For example, when (a3) is zero, Y₁₇ is not linked directly to Y₁₄.

For purposes of the present invention, when values for bifunctional linkers are positive integers equal to or greater than 2, the same or different bifunctional linkers can be employed.

R₂₁-R₂₈, in each occurrence, are independently the same or different when each of (t1), (t2), (t3) and (t4) is independently equal to or greater than 2.

In one embodiment, Y₁₄₋₁₅ and Y₁₇₋₁₉ are O or NH; and R₂₁₋₂₉ are independently hydrogen or methyl.

In another embodiment, Y₁₆ is O; Y₁₄₋₁₅ and Y₁₇₋₁₉ are O or NH; and R₂₁₋₂₉ are hydrogen.

In certain embodiments, L₁ is independently selected from among:

—(CH₂)ₜ₁—[C(═O)]ₐ₃—,

—(CH₂)ₜ₁Y₁₇—(CH₂)ₜ₂—(Y₁₈)ₐ₂—[C(═O)]ₐ₃—,

—(CH₂CH₂Y₁₇)ₜ₁—[C(═O)]ₐ₃—,

—(CH₂CH₂Y₁₇)ₜ₁(CH₂)ₜ₄—(Y₁₈)ₐ₂—[C(═O)]ₐ₃—,

—[(CH₂CH₂)ₜ₂Y₁₇]ₜ₃(CH₂)ₜ₄—(Y₁₈)ₐ₂—[C(═O)]ₐ₃—,

—(CH₂)ₜ₁—[(CH₂)ₜ₂Y₁₇]ₜ₃ (CH₂)ₜ₄—(Y₁₈)ₐ₂—[C(═O)]ₐ₃—,

—(CH₂)ₜ₁(Y₁₇)ₐ₂[C(═O)]ₐ₃(CH₂)ₜ₂—,

—(CH₂)ₜ₁(Y₁₇)ₐ₂[C(═O)]ₐ₃Y₁₄(CH₂)ₜ₂—,

—(CH₂)ₜ₁(Y₁₇)ₐ₂[C(═O)]ₐ₃(CH₂)ₜ₂—Y₁₅—(CH₂)ₜ₃—,

—(CH₂)ₜ₁(Y₁₇)ₐ₂[C(═O)]ₐ₃Y₁₄(CH₂)ₜ₂—Y₁₅—(CH₂)ₜ₃—,

—(CH₂)ₜ₁(Y₁₇)ₐ₂[C(═O)]ₐ₃(CH₂CH₂Y₁₉)ₜ₂(CH₂)ₜ₃—, and

—(CH₂)ₜ₁(Y₁₇)ₐ₂[C(═O)]ₐ₃Y₁₄(CH₂CH₂Y₁₉)ₜ₂(CH₂)ₜ₃—,

wherein

Y₁₄₋₁₅ and Y₁₇₋₁₉ are independently O, or NH;

(t1), (t2), (t3), and (t4) are independently zero or positive integers, preferably zero or positive integers of from about 1 to about 10 (e.g., 1, 2, 3, 4, 5, 6); and

(a2) and (a3) are independently zero or 1.

Y₁₇, in each occurrence, is the same or different, when (t1) or (t3) is equal to or greater than 2.

Y₁₉, in each occurrence, is the same or different, when (t2) is equal to or greater than 2.

In a further embodiment and/or alternative embodiments, illustrative examples of the L₁ group are selected from among:

—CH₂—, —(CH₂)₂—, —(CH₂)₃—, —(CH₂)₄—, —(CH₂)₅—, —(CH₂)₆—, —NH(CH₂)—,

—CH(NH₂)CH₂—,

—(CH₂)₄—C(═O)—, —(CH₂)₅—C(═O)—, —(CH₂)₆—C(═O)—,

—CH₂CH₂O—CH₂O—C(═O)—,

—(CH₂CH₂O)₂—CH₂O—C(═O)—,

—(CH₂CH₂O)₃—CH₂O—C(═O)—,

—(CH₂CH₂O)₂—C(═O)—,

—CH₂CH₂O—CH₂CH₂NH—C(═O)—,

—(CH₂CH₂O)₂—CR₂CH₂NH—C(═O)—,

—CH₂—O—CH₂CH₂O—CH₂CH₂NH—C(═O)—,

—CH₂—O—(CH₂CH₂O)₂—CH₂CH₂NH—C(═O)—,

—CH₂—O—CH₂CH₂O—CH₂C(═O)—,

—CH₂—O—(CH₂CH₂O)₂—CH₂C(═O)—,

—(CH₂)₄—C(═O)NH—, —(CH₂)₅—C(═O)NH—,

—(CH₂)₆—C(═O)NH—,

—CH₂CH₂O—CH₂O—C(═O)—NH—,

—(CH₂CH₂O)₂—CH₂O—C(═O)—NH—,

—(CH₂CH₂O)₃—CH₂O—C(═O)—NH—,

—(CH₂CH₂O)₂—C(═O)—NH—,

—CH₂CH₂O—CH₂CH₂NH—C(═O)—NH—,

—(CH₂CH₂O)₂—CH₂CH₂NH—C(═O)—NH—,

—CH₂—O—CH₂CH₂O—CH₂NH—C(═O)—NH—,

—CH₂—O——(CH₂CH₂O)₂—CH₂CH₂NH—C(═O)—NH—,

—CH₂—O—CH₂CH₂O—CH₂C(═O)—NH—,

—CH₂—O—(CH₂CH₂O)₂—CH₂C(═O)—NH—,

—(CH₂CH₂O)₂—, —CH₂CH₂O—CH₂O—,

—(CH₂CH₂O)₂—CH₂CH₂NH—,

—(CH₂CH₂O)₃—CH₂CH₂NH—,

—CH₂CH₂O—CH₂CH₂NH—,

—(CH₂CH₂O)₂—CH₂CH₂NH—,

—CH₂—O—CH₂CH₂O—CH₂CH₂NH—,

—CH₂—O—(CH₂CH₂O)₂—CH₂CH₂NH—,

—CH₂—O—CH₂CH₂O—,

—CH₂—O—(CH₂CH₂O)₂—,

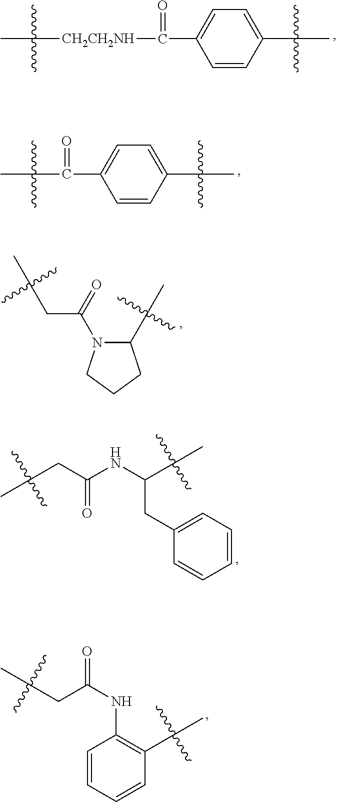

—C(═O)NH(CH₂)₂—, —CH₂C(═O)NH(CH₂)₂—,

—C(═O)NH(CH₂)₃—, —CH₂C(═O)NH(CH₂)₃—,

—C(═O)NH(CH₂)₄—, —CH₂C(═O)NH(CH₂)₄—,

—C(═O)NH(CH₂)₅—, —CH₂C(═O)NH(CH₂)₅—,

—C(═O)NH(CH₂)₆—, —CH₂C(═O)NH(CH₂)₆—,

—C(═O)O(CH₂)₂—, —CH₂C(═O)O(CH₂)₂—,

—C(═O)O(CH₂)₃—, —CH₂C(═O)O(CH₂)₃—,

—C(═O)O(CH₂)₄—, —CH₂C(═O)O(CH₂)₄—,

—C(═O)O(CH₂)₅—, —CH₂C(═O)O(CH₂)₅—,

—C(═O)O(CH₂)₆—, —CH₂C(═O)O(CH₂)₆—,

—(CH₂CH₂)₂NHC(═O)NH(CH₂)₂—,

—(CH₂CH₂)₂NHC(═O)NH(CH₂)₃—,

—(CH₂CH₂)₂NHC(═O)NH(CH₂)₄—,

—(CH₂CH₂)₂NHC(═O)NH(CH₂)₅—,

—(CH₂CH₂)₂NHC(═O)NH(CH₂)₆—,

—(CH₂CH₂)₂NHC(═O)O(CH₂)₂—,

—(CH₂CH₂)₂NHC(═O)O(CH₂)₃—,

—(CH₂CH₂)₂NHC(═O)O(CH₂)₄—,

—(CH₂CH₂)₂NHC(═O)O(CH₂)₅—,

—(CH₂CH₂)₂NHC(═O)O(CH₂)₆—,

—(CH₂CH₂)₂NHC(═O)(CH₂)₂—,

—(CH₂CH₂)₂NHC(═O)(CH₂)₃—,

—(CH₂CH₂)₂NHC(═O)(CH₂)₄—,

—(CH₂CH₂)₂NHC(═O)(CH₂)₅—, and

—(CH₂CH₂)₂NHC(═O)(CH₂)₆—.

In certain embodiments, L₂ is independently selected from among:

—(CR′₂₁R′₂₂)_{t′1}—[C(═Y′₁₆)]_{a′3}(CR′₂₇CR′₂₈)ₜ₂—,

—(CR′₂₁R′₁₂)_{t′1}Y′₁₄—(CR′₂₃R′₂₄)_{t′2}—(Y′₁₅)_{a′2}—[C(═Y′₁₆)]_{a′3}(CR′₂₇CR′₂₈)_{t′3}—,

—(CR′₂₁R′₂₂CR′₂₃R′₂₄Y′₁₄)_{t′1}—[C_{(═Y′}₁₆)]_{a′3}(CR′₂₇CR′₂₈)_{t′2}—,

—(CR′₂₁R′₂₂CR′₂₃R′₂₄Y′₁₄)_{t′1}(CR′₂₅R′₂₆)_{t′2}—(Y′₁₅)_{a′2}—[C(═Y′₁₆)]_{a′3}(CR′₂₇CR′₂₈)_{t′3}—,

—(CR′₂₁R′₂₂CR′₂₃R′₂₄)_{t′2}Y′₁₄]_{t′1}(CR′₂₅R′₂₆)_{t′2}—(Y′₁₅)_{a′2}—[C(═Y′₁₆)]_{a′3}(CR′₂₇CR′₂₈)_{t′3}—,

—(CR′₂₁R′₂₂)_{t′1}—[(CR′₂₃R′₂₄)_{t′2}Y′₁₄]_{t′2}(CR′₂₅R′₂₆)_{t′3}—(Y′₁₅)_{a′2}—[C(═Y′₁₆)]_{a′3}(CR′₂₇CR′₂₈)_{t′4}—

—(CR′₂₁R′₂₂)_{t′1}(Y′₁₄)_{a′2}[C(═Y′₁₆)]_{a′3}(CR′₂₃R′₂₄)_{t′2}—,

—(CR′₂₁R′₂₂)_{t′1}(Y′₁₄)_{a′2}[C(═Y′₁₆)]_{a′3}Y′₁₅(CR′_{≦}R′₂₄)_{t′2}—,

—(CR′₂₁R′₂₂)_{t′1}(Y′₁₄)_{a′2}[C(═Y′₁₆)]_{a′3}(CR′₂₃R′₂₄)_{t′2}—Y′₁₅—(CR′₂₃R′₂₄)_{t′3}—,

—(CR′₂₁R′₂₂)_{t′1}(Y′₁₄)_{a′2}[C(═Y′₁₆)]_{a′3}Y′₁₄(CR′₂₃R′₂₄)_{t′2}—Y′₁₅—(CR′₂₃R′₂₄)_{t′3}—,

—(CR′₂₁R′₂₂)_{t′1}(Y′₁₄)_{a′2}[C(═Y′₁₆)]_{a′3}(CR′₂₃R′₂₄CR′₂₅R′₂₆Y′₁₅)_{t′2}(CR′₂₇CR′₂₈)_{t′3—,}

—(CR′₂₁R′₂₂)_{t′1}(Y′₁₄)_{a′2}[C(═Y′₁₆)]_{a′3}Y′₁₇(CR′₂₃R′₂₄CR′₂₅R′₂₆Y′₁₅)_{t′2}(CR′₂₇CR′₂₈)_{t′3}—, and

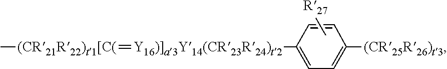

wherein:

Y′₁₆ is O, NR′₂₈, or S, preferably oxygen;

Y′₁₄₋₁₅ and Y′₁₇ are independently O, NR′₂₉, or S, preferably O, or NR′₂₉;

R′₂₁₋₂₇ are independently selected from among hydrogen, hydroxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆heteroalkyls, C₁₋₆ alkoxy, phenoxy and C₁₋₆ heteroalkoxy, preferably, hydrogen, methyl, ethyl or propyl;

R′₂₈₋₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆heteroalkyls, C₁₋₆ alkoxy, phenoxy and C₁₋₆ heteroalkoxy, preferably, hydrogen, methyl, ethyl or propyl;

(t′1), (t′2), (t′3) and (t′4) are independently zero or positive integers, preferably zero or a positive integer of from about 1 to about 10 (e.g., 1, 2, 3, 4, 5, 6); and

(a′2) and (a′3) are independently zero or 1.

The bifunctional L₂ linkers contemplated within the scope of the present invention include those in which combinations of variables and substituents of the linkers groups are permissible so that such combinations result in stable compounds of Formula (I). For example, when (a′3) is zero, Y′₁₄ is not linked directly to Y′₁₄ or Y′₁₇.

For purposes of the present invention, when values for bifunctional L₂ linkers including releasable linkers are positive integers equal to or greater than 2, the same or different bifunctional linkers can be employed.

In one embodiment, Y′₁₄₋₁₅ and Y′₁₇ are O or NH; and R′₂₁₋₂₉ are independently hydrogen or methyl.

In another embodiment, Y′₁₆ is O; Y′₁₄₋₁₅ and Y′₁₇ are O or NH; and R′₂₁₋₂₉ are hydrogen.

In certain embodiments, L₂ is selected from among:

—(CH₂)_{t′1}—[C(═O)]_{a′3}(CH₂)_{t′2}—,

—(CH₂)_{t′1}Y′₁₄—(CH₂)_{t′2}—(Y′₁₅)_{a′2}—[C(═O)]_{a′3}(CH₂)_{t′3}—,

—(CH₂CH₂Y′₁₄)_{t′1}—[C(═O)]_{a′3}(CH₂)_{t′2}—,

—(CH₂CH₂Y′₁₄)_{t′1}(CH₂)_{t′2}—[C(═O)]_{a′3}(CH₂)_{t′3}—,

—[(CH₂CH₂)_{t′2}Y₁₄]_{t′1}(CH₂)_{t′2}—(Y′₁₅)_{a′2}—[C(═O)]_{a′3}(CH₂)_{t′3}—,

—(CH₂)_{t′1}—[(CH₂)_{t′2}(CH₂)_{t′3}—(Y′₁₅)_{a′2}—[C(═O)]_{a′3}(CH₂)_{t′4}—,

—(CH₂)_{t′1}(Y′₁₄)_{a′2}[C(═O)]_{a′3}(CH₂)_{t′2}—,

—(CH₂)_{t′1}(Y′₁₄)_{a′2}[C(═O)]_{a′3}Y′₁₅(CH₂)_{t′2}—,

—(CH₂)_{t′1}(Y′₁₄)_{a′2}[C(═O)]_{a′3}(CH₂)_{t′2}—Y′₁₅—(CH₂)_{t′3}—,

—(CH₂)_{t′1}(Y′₁₄)_{a′2}[C(═O)]_{a′3}Y′₁₄(CH₂)_{t′2}—Y′₁₅—(CH₂)_{t′3}—,

—(CH₂)_{t′1}(Y′₁₄)_{a′2}[C(═O)]_{a′3}(CH₂CH₂Y′₁₅)_{t′2}(CH₂)_{t′3}—, and

—(CH₂)_{t′1}(Y′₁₄)_{a′2}[C(═O)]_{a′3}(CH₂CH₂Y′₁₅)_{t′2}(CH₂)_{t′3}—,

wherein

Y′₁₄₋₁₅ and Y′₁₇ are independently O, or NH;

(t′1), (t′2), (t′3), and (t′4) are independently zero or positive integers, preferably 0 or positive integers of from about 1 to about 10 (e.g., 1, 2, 3, 4, 5, 6); and

(a′2) and (a′3) are independently zero or 1.

Y^{′}₁₄, in each occurrence, is the same or different, when (t′1) or (t′2) is equal to or greater than 2.

Y′₁₅, in each occurrence, is the same or different, when (t′2) is equal to or greater than 2.

In a further embodiment and/or alternative embodiments, illustrative examples of the L₂ group are selected from among:

—CH₂—, —(CH₂)₂—, —(CH₂)₃—, —(CH₂)₄—, —(CH₂)₅—, —(CH₂)₆—, —NH(CH₂)—,

—CH(NH₂)CH₂—,

—O(CH₂)₂—, —C(═O)O(CH₂)₃—, —C(═O)NH(CH₂)₃—,

—C(═O) (CH₂)₂—, —C(═O)(CH₂)₃—,

—CH₂—C(═O)—O(CH₂)₃—,

—CH₂—C(═O)—NH(CH₂)₃—,

—CH₂—OC(═O)—O(CH₂)₃—,

—CH₂—OC(═O)—NH(CH₂)₃—,

—(CH₂)₂—C(═O)—O(CH₂)₃—,

—(CH₂)₂—C(═O)—NH(CH₂)₃—,

—CH₂C(═O)O(CH₂)₂—O—(CH₂)₂—,

—CH₂C(═O)NH(CH₂)₂—O—(CH₂)₂—,

—(CH₂)₂C(═O)O(CH₂)₂—O—(CH₂)₂—,

—(CH₂)₂C(═O)NH(CH₂)₂—O—(CH₂)₂—,

—CH₂C(═O)O(CH₂CH₂O)₂CH₂CH₂—,

—(CH₂)₂C(═O)O(CH₂CH₂O)₂CH₂CH₂—,

—(CH₂CH₂O)₂—, —CH₂CH₂O—CH₂O—

—(CH₂CH₂O)₂—CH₂CH₂NH—, —(CH₂CH₂O)₃—CH₂CH₂NH—,

—CH₂CH₂O—CH₂CH₂NH—,

—CH₂—O—CH₂CH₂O—CH₂CH₂NH—,

—CH₂—O—(CH₂CH₂O)₂—CH₂CH₂NH—,

—CH₂—O—CH₂CH₂O—, —CH₂—O—(CH₂CH₂O)₂—,

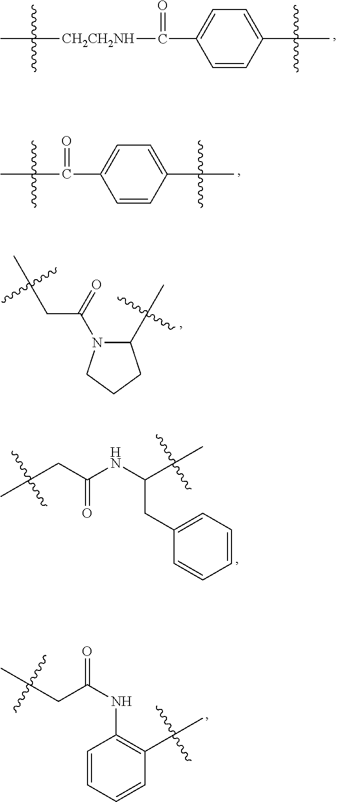

—(CH₂)₂NHC(═O)—(CH₂CH₂O)₂—,

—C(═O)NH(CH₂)₂—, —CH₂C(═O)NH(CH₂)₂—,

—C(═O)NH(CH₂)₃—, —CH₂C(═O)NH(CH₂)₃—,

—C(═O)NH(CH₂)₄—, —CH₂C(═O)NH(CH₂)₄—,

—C(═O)NH(CH₂)₅—, —CH₂C(═O)NH(CH₂)₅—,

—C(═O)NH(CH₂)₆—, —CH₂C(═O)NH(CH₂)₆—,

—C(═O)O(CH₂)₂—, —CH₂C(═O)O(CH₂)₂—,

—C(═O)O(CH₂)₃—, —CH₂C(═O)O(CH₂)₃—,

—C(═O)O(CH₂)₄—, —CH₂C(═O)O(CH₂)₄—,

—C(═O)O(CH₂)₅—, —CH₂C(═O)O(CH₂)₅—,

—C(═O)O(CH₂)₆—, —CH₂C(═O)O(CH₂)₆—,

—(CH₂CH₂)₂NHC(═O)NH(CH₂)₂—,

—(CH₂CH₂)₂NHC(═O)NH(CH₂)₃—,

—(CH₂CH₂)₂NHC(═O)NH(CH₂)₄—,

—(CH₂CH₂)₂NHC(═O)NH(CH₂)₅—,

—(CH₂CH₂)₂NHC(═O)NH(CH₂)₆—,

—(CH₂CH₂)₂NHC(═O)O(CH₂)₂—,

—(CH₂CH₂)₂NHC(═O)O(CH₂)₃—,

—(CH₂CH₂)₂NHC(═O)O(CH₂)₄—,

—(CH₂CH₂)₂NHC(═O)O(CH₂)₅—,

—(CH₂CH₂)₂NHC(═O)O(CH₂)₆—,

—(CH₂CH₂)₂NHC(═O)(CH₂)₂—,

—(CH₂CH₂)₂NHC(═O)(CH₂)₃—,

—(CH₂CH₂)₂NHC(═O)(CH₂)₄—,

—(CH₂CH₂)₂NHC(═O)(CH₂)₅—, and

—(CH₂CH₂)₂NHC(═O)(CH₂)₆—.

In a further embodiment, the bifunctional linkers L₁ and L₂ can be a spacer having a substituted saturated or unsaturated, branched or linear, C₃₋₅₀ alkyl (i.e., C₃₋₄₀ alkyl, C₃₋₂₀ alkyl, C₃₋₁₅ alkyl, C₃₋₁₀ alkyl, etc.), wherein optionally one or more carbons are replaced with NR₆, O, S or C(═Y), (preferably O or NH), but not exceeding 70% (i.e., less than 60%, 50%, 40%, 30%, 20%, 10%) of the carbons being replaced.

### 4. The Bifunctional Spacers: L₁₁, L₁₂ and L₁₃ Groups

According to the present invention, the bifunctional spacers L₁₁₋₁₃are independently selected from among:

—(CR₃₁R₃₂)_{q1}—; and

—Y₂₆(CR₃₁R₃₂)_{q1}—,

wherein:

Y₂₆ is O, NR₃₃, or S, preferably oxygen or NR₃₃;

R₃₁₋₃₂ are independently selected from among hydrogen, hydroxyl, C₁₋₆ alkyls, C₃₋₁₂ branched alkyls, C₃₋₈ cycloalkyls, C₁₋₆ substituted alkyls, C₃₋₈ substituted cycloalkyls, C₁₋₆ heteroalkyls, substituted C₁₋₆ heteroalkyls, C₁₋₆ alkoxy, phenoxy and C₁₋₆ heteroalkoxy, preferably, hydrogen, methyl, ethyl or propyl;

R₃₃ is selected from among hydrogen, hydroxyl, C₁₋₆ alkyls, C₃₋₁₂ branched alkyls, C₃₋₈ cycloalkyls, C₁₋₆ substituted alkyls, C₃₋₈ substituted cycloalkyls, C₁₋₆ heteroalkyls, substituted C₁₋₆ heteroalkyls, C₁₋₆ alkoxy, phenoxy and C₁₋₆heteroalkoxy, preferably, hydrogen, methyl, ethyl or propyl; and

(q1) is zero or a positive integer, preferably zero or an integer of from about 1 to about 10 (e.g., 1, 2, 3, 4, 5, 6).

The bifunctional spacers contemplated within the scope of the present invention include those in which combinations of substituents and variables are permissible so that such combinations result in stable compounds of Formula (I).

R₃₁ and R₃₂, in each occurrence, are independently the same or different when (q1) is equal to or greater than 2.

In one preferred embodiment, R′₃₁₋₃₃ are hydrogen or methyl.

In certain preferred embodiments, R₃₁₋₃₂ are hydrogen or methyl; and Y₂₆ is O or NH.

The C(R₃₁)(R₃₂) moiety is the same or different when (q1) is equal to or greater than 2.

In a further and/or alternative embodiments, L₁₁₋₁₃ are independently selected from among:

—CH₂—, —(CH₂)₂—, —(CH₂)₃—, —(CH₂)—, —(CH₂)₅—, —(CH₂)₆—,

—(CH₂)₂—, —O(CH₂)₃—, —O(CH₂)₄—, —O(CH₂)₅—, —O(CH₂)₆—, CH(OH)—,

—(CH₂CH₂O)—CH₂CH₂—,

—(CH₂CH₂O)₂—CH₂CH₂—,

—C(═O)O(CH₂)₃—, —C(═O)NH(CH₂)₃—,

—C(═O)(CH₂)₂—, —C(═O)(CH₂)₃—,

—CH₂—C(═O)—O(CH₂)₃—,

—CH₂—C(═O)—NH(CH₂)₃—,

—CH₂—OC(═O)—O(CH₂)₃—,

—CH₂—OC(═O)—NH(CH₂)₃—,

—(CH₂)₂—C(═O)—O(CH₂)₃—,

—(CH₂)₂—C(═O)—NH(CH₂)₃—,

—CH₂C(═O)O(CH₂)₂—O—(CH₂)₂—,

—CH₂C(═O)NH(CH₂)₂—O—(CH₂)₂—,

—(CH₂)₂C(═O)O(CH₂)₂—O—(CH₂)₂—,

—(CH₂)₂C(═O)NH(CH₂)₂—O—(CH₂)₂—,

—CH₂C(═O)O(CH₂CH₂O)₂CH₂CH₂—, and

—(CH₂)₂C(═O)O(CH₂CH₂O)₂CH₂CH₂—.

### 5. The Q Group

According to the present invention, the Q group contains one or more substituted or unsubstituted, saturated or unsaturated C4-30-containing moieties. The Q group includes one or more C4-30 aliphatic saturated or unsaturated hydrocarbons.

The Q group is represented by Formula (Ia):

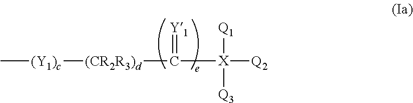

wherein

X is C, N or P;

Q₁ is H, C₁₋₃ alkyl, NR₅, OH, or

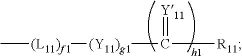

Q₂ is H, C₁₋₃ alkyl, NR₆, OH, or

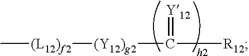

Q₃ is a lone electron pair, (═O), H, C₁₋₃ alkyl, NR₇, OH, or

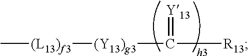

L₁₁, L₁₂ and L₁₃ are independently selected bifunctional spacers;

Y₁₁, Y′₁₁, Y₁₂, Y′₁₂, Y₁₃, and Y′₁₃ are independently O, S or NR₈;

R₁₁, R₁₂ and R₁₃ are independently (substituted or unsubstituted) saturated or unsaturated C₄₋₃₀; and

all other variables are as defined above,

provided that Q includes at least one or two of R₁₁, R₁₂ and R₁₃.

In one preferred embodiment, R₁₁, R₁₂ and R₁₃ independently include a C4-30 saturated or unsaturated aliphatic hydrocarbon. More preferably, each aliphatic hydrocarbon is a saturated or unsaturated C8-24 hydrocarbon (yet more preferably, C12-22 hydrocarbon: C12-22 alkyl, C12-22 alkenyl, C12-22 alkyloxy). Examples of aliphatic hydrocarbon include, but are not limited to, auroyl (C12), myristoyl (C14), palmitoyl (C16), stearoyl (C18), oleoyl (C18), and erucoyl (C22); saturated or unsaturated C12 alkyloxy, C14 alkyloxy, C16 alkyloxy, C18 alkyloxy, C20 alkyloxy, and C22 alkyloxy; and, saturated or unsaturated C12 alkyl, C14 alkyl, C16 alkyl, C18 alkyl, C20 alkyl, and C22 alkyl,

Preferably, at least two of R₁₁, R₁₂ and R₁₃ independently include a saturated or unsaturated C8-24 hydrocarbon (more preferably, C12-22 hydrocarbon).

Some examples of Q group are represented by the formula:

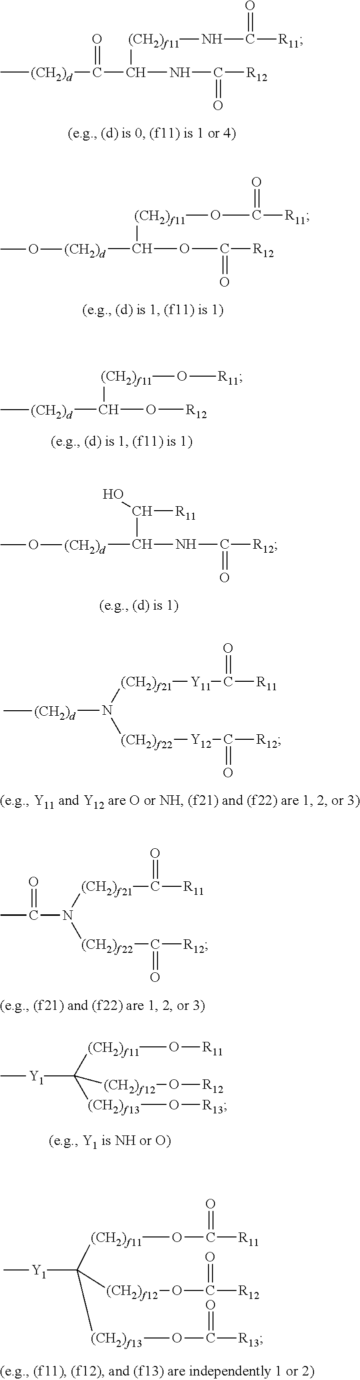

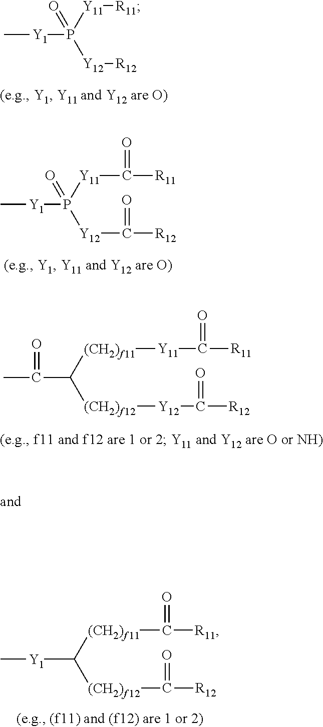

wherein,

Y₁ is O, S, or NR₃₁, preferably oxygen or NH;

R₁₁, R₁₂, and R₁₃ are independently substituted or unsubstituted, saturated or unsaturated C₄₋₃₀ (alkyl, alkenyl, alkoxy);

R₃₁ is hydrogen, methyl or ethyl;

(d) is 0 or a positive integer, preferably 0 or an integer from about”(to about 10 (e.g., 1, 2, 3, 4, 5, 6);

(f11), (f12) and (f13) are independently 0, 1, 2, 3, or 4; and

(f21) and (f22) are independently 1, 2, 3 or 4.

In certain embodiments, the Q group includes diacylglycerol, diacylglycamide, dialkylpropyl, phosphatidylethanolamine or ceramide. Suitable diacylglycerol or diacylglycamide include a dialkylglycerol or dialkylglycamide group having alkyl chain length independently containing from about C₄ to about C₃₀, preferably from about C₈ to about C₂₄, saturated or unsaturated carbon atoms. The dialkylglycerol or dialkylglycamide group can further include one or more substituted alkyl groups.

The term “diacylglycerol” (DAG) used herein refers to a compound having two fatty acyl chains, R₁₁₁ and R₁₁₂. The R₁₁ and R₁₂ have the same or different about 4 to about 30 carbons (preferably about 8 to about 24) and are bonded to glycerol by ester linkages. The acyl groups can be saturated or unsaturated with various degrees of unsaturation. DAG has the general formula:

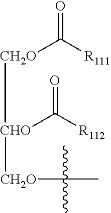

Examples of the DAG can be selected from among a dilaurylglycerol (C12), a dimyristylglycerol (C14, DMG), a dipalmitoylglycerol (C16, DPG), a distearylglycerol (C18, DSG), a dioleoylglycerol (C18), a dierucoyl (C22), a dilaurylglycamide (C12), a dimyristylglycamide (C14), a dipalmitoylglycamide (C16), a disterylglycamide (C18), a dioleoylglycamide (C18), dierucoylglycamide (C22). Those of skill in the art will readily appreciate that other diacylglycerols are also contemplated.

The term “dialkyloxypropyl” refers to a compound having two alkyl chains, R₁₁₁ and R₁₁₂. The R₁₁₁ and R₁₁₂ alkyl groups include the same or different between about 4 to about 30 carbons (preferably about 8 to about 24). The alkyl groups can be saturated or have varying degrees of unsaturation. Dialkyloxypropyls have the general formula:

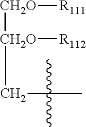

wherein R₁₁₁ and R₁₁₂ alkyl groups are the same or different alkyl groups having from about 4 to about 30 carbons (preferably about 8 to about 24). The alkyl groups can be saturated or unsaturated. Suitable alkyl groups include, but are not limited to, lauryl (C12), myristyl (C14), palmityl (C16), stearyl (C18), oleoyl (C18) and icosyl (C20).

In one embodiment, R₁₁₁ and R₁₁₂ are both the same, i.e., R₁₁₁ and R₁₁₂ are both myristyl (C14) or both oleoyl (C18), etc. In another embodiment, R₁₁₁ and 8₁₁₂ are different, i.e., R₁₁₁ is myristyl (C14) and R₁₁₂ is stearyl (C18).

In another embodiment, the Q group can include phosphatidylethanolamines (PE). The phosphatidylethanolamines useful for the releasable fusogenic lipid conjugation can contain saturated or unsaturated fatty acids with carbon chain lengths in the range of about 4 to about 30 carbons (preferably about 8 to about 24). Suitable phosphatidylethanolamines include, but are not limited to: dimyristoylphosphatidylethanolamine (DMPE), dipalmitoylphosphatidylethanolamine (DPPE), dioleoylphosphatidylethanolamine (DOPE) and distearoylphosphatidylethanolamine (DSPE).

In yet another embodiment, the Q group can include ceramides (Cer). Ceramides have only one acyl group. Ceramides can have saturated or unsaturated fatty acids with carbon chain lengths in the range of about 4 to about 30 carbons (preferably about 8 to about 24).

One preferred embodiment includes:

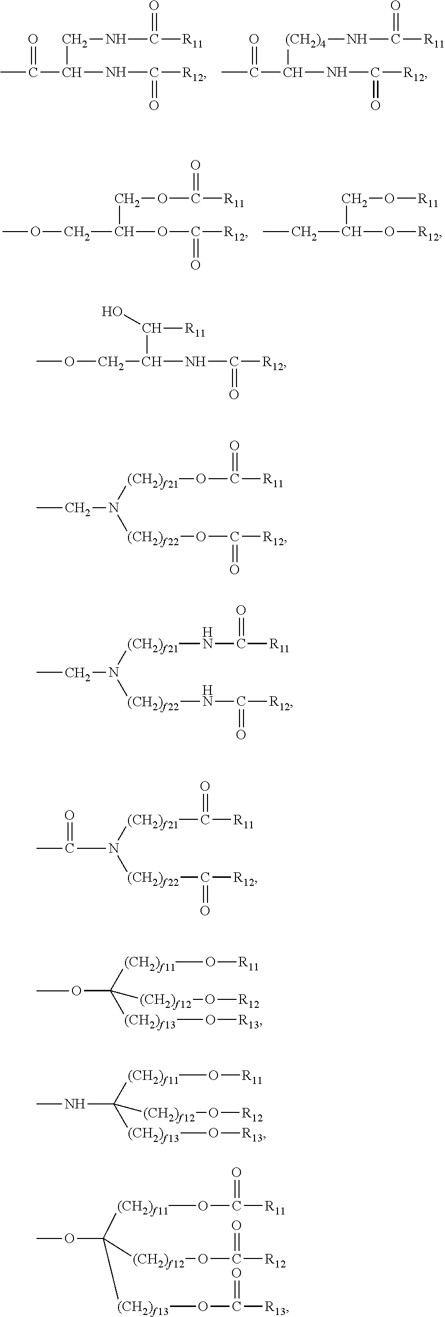

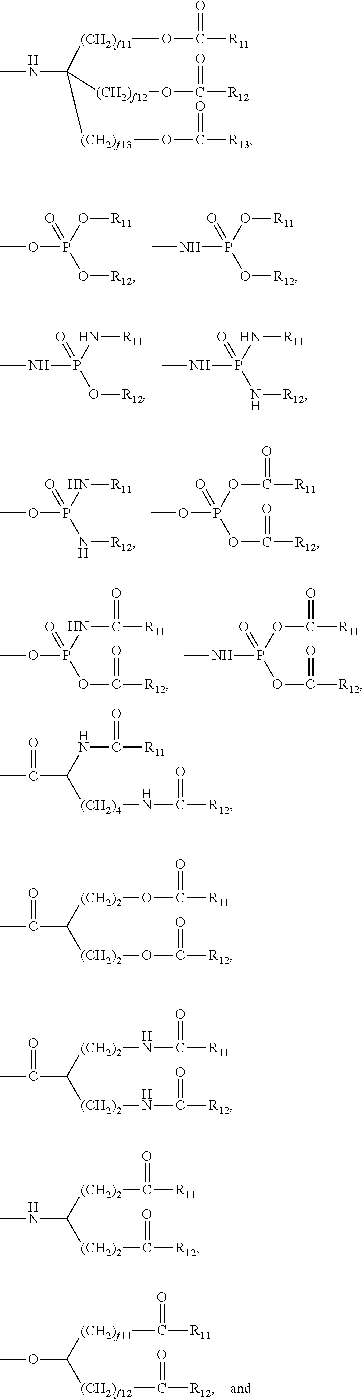

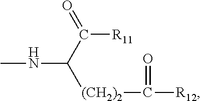

wherein R₁₁-₁₃ are independently the same or different C12-22 saturated or unsaturated aliphatic hydrocarbons such as a dilauryl (C12), a dimyristyl (C14), a dipalmitoyl (C16), a distearyl (C18), a dioleoyl (C18), and a dierucoyl (C22);

(f11), (f12) and (f13) are independently 0, 1, 2, 3, or 4; and

(f21) and (f22) are independently 1, 2, 3 or 4.

### B. Preparation of Releasable Fusogenic Lipids of Formula (1)

Synthesis of representative, specific compounds, is set forth in the Examples. Generally, however, the compounds of the present invention can be prepared in several fashions. According to the present invention, the methods of preparing compound of Formula (I) described herein include reacting an amine-containing compound with an aldehyde-containing compound to provide a fusogenic lipid having an imine moiety. The amine can be a primary amine and the aldehyde can further contains aliphatic or aromatic substituents.

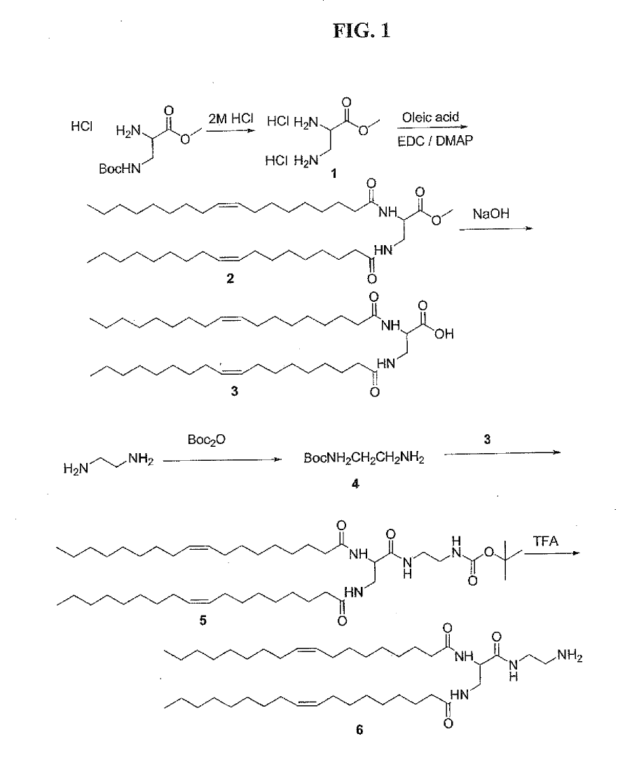

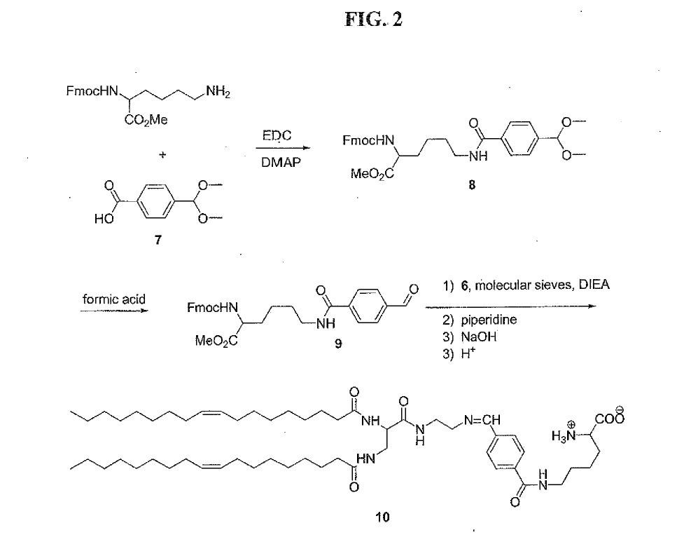

One representative example of the preparation of fusogenic lipid is shown in FIG. 1 and FIG. 2. First, lipids are coupled with a nucleophilic multifunctional linker (compound 1) to provide compound 2 in the presence of a coupling agent such as EDC or DIPC. Preferably, the reaction is carried out in an inert solvent such as methylene chloride, chloroform, toluene, DMF or mixtures thereof. The reaction is also preferably conducted in the presence of a base, such as DMAP, DIEA, pyridine, triethylamine, etc. at a temperature of from −4° C. to about 70° C. (e.g. −4° C. to about 50° C.). In one preferred embodiment, the reaction is performed at a temperature from 0° C. to about 25° C. or 0° C. to about room temperature.

The terminal functional group of compound 2 is further coupled with a bifunctional linker, such as compound 4, followed by removal of an amine protecting group to provide a lipid compound having a terminal amine (compound 6).

A compound containing zwitterionic moieties, such as Fmoc-Lys(OMe)-NH₂, is reacted with a bifunctional linker, such as compound 7, to provide compound 8 with a protected aldehyde. The aldehyde protecting group is removed. The aldehyde of compound 9 is reacted with an amine-containing lipid (compound 6) under conditions for dehydration, followed by removal of amine protecting group and saponification to provide fusogenic lipids containing an imine bond.

Attachment of the lipids to the nucleophilic multifunctional linker can be carried out using standard organic synthetic techniques in the presence of a base, using coupling agents known to those of ordinary skill in the art such as 1,3-diisopropylcarbodiimide (DIPC), dialkyl carbodiimides, 2-halo-1-alkylpyridinium halides, 1-(3-dimethylaminopropyl)-3-ethyl carbodiimide (EDC), propane phosphonic acid cyclic anhydride (PPACA) and phenyl dichlorophosphates. In addition, the formation of imine bond can be carried out using standard organic synthetic techniques for dehydration, such as using molecular sieves, azeotrophing, acid-catalyzed dehydration, etc.

In another embodiment, an activated lipid acid, such as NHS or PNP ester, can be used to react with the nucleophile multifunctional linker, such as compound 1.

Alternatively, when lipids are activated with a leaving group such as NHS, or PNP, a coupling agent is not required and the reaction proceeds in the presence of a base.

Removal of a protecting group from an amine-containing compound can be carried out with a strong acid such as trifluoroacetic acid (TFA), HCl, sulfuric acid, etc., or catalytic hydrogenation, radical reaction, etc. Alternatively, removal of an amine-protecting group, such as Fmoc, can be carried out with a base such as piperidine or DMAP. In one preferred embodiment, the deprotection of Boc group is carried out with HCl solution in dioxane. The deprotection reaction can be carried out at a temperature from −4° C. to about 50° C. Preferably, the reaction is carried out at a temperature from 0° C. to about 25° C. or to room temperature. In more preferred embodiment, the deprotection of Boc group is carried out at room temperature.

For example, compounds prepared by the methods described herein include:

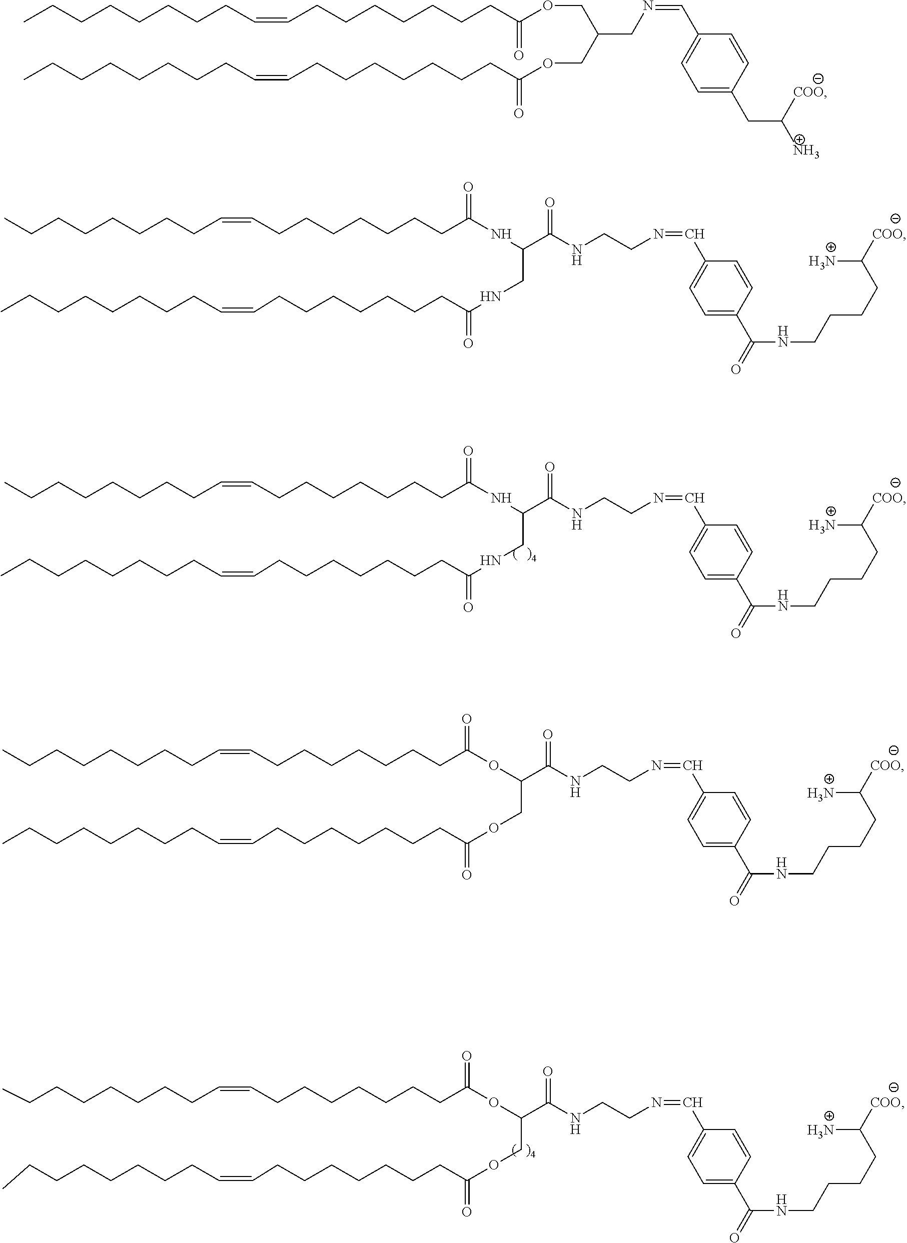

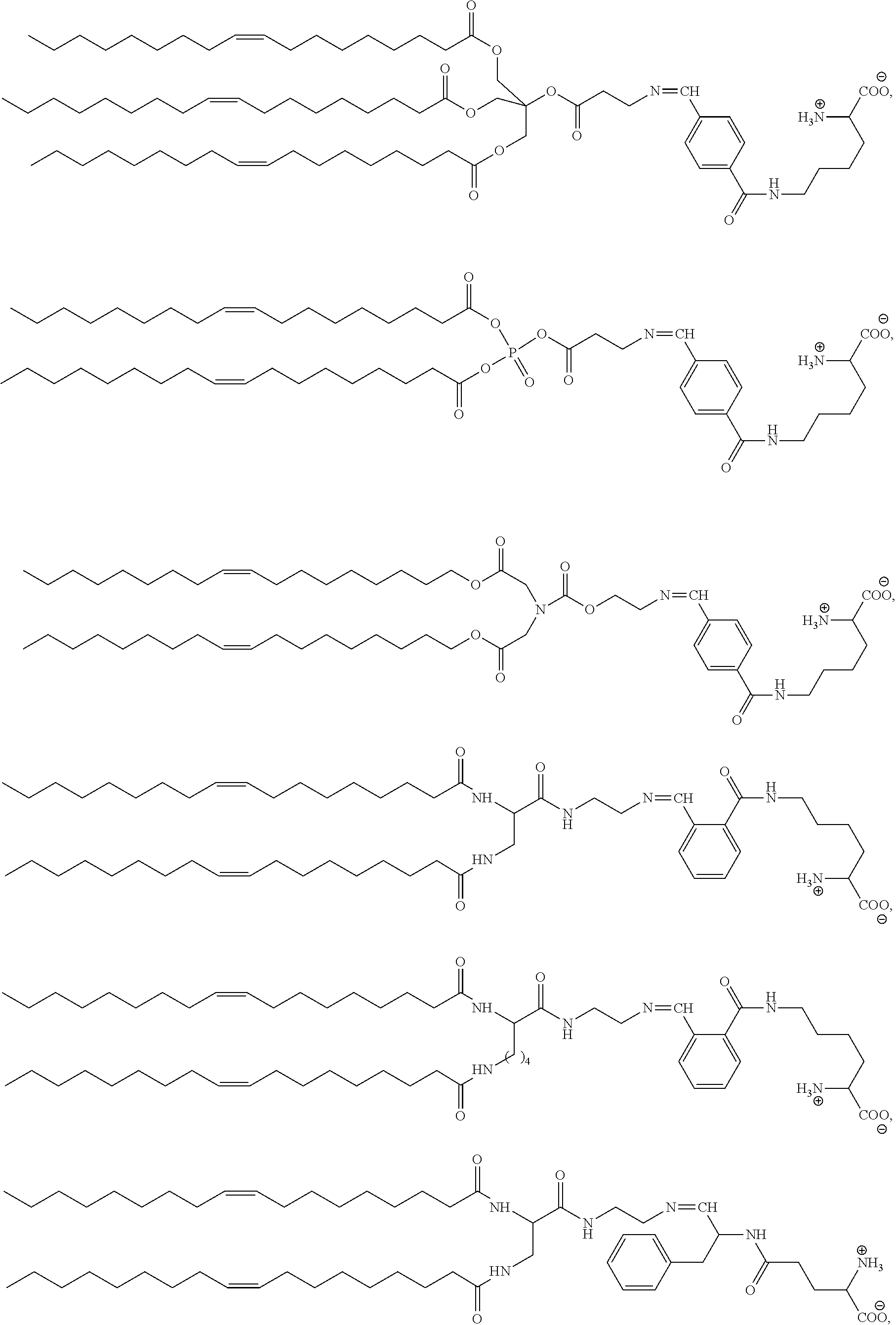

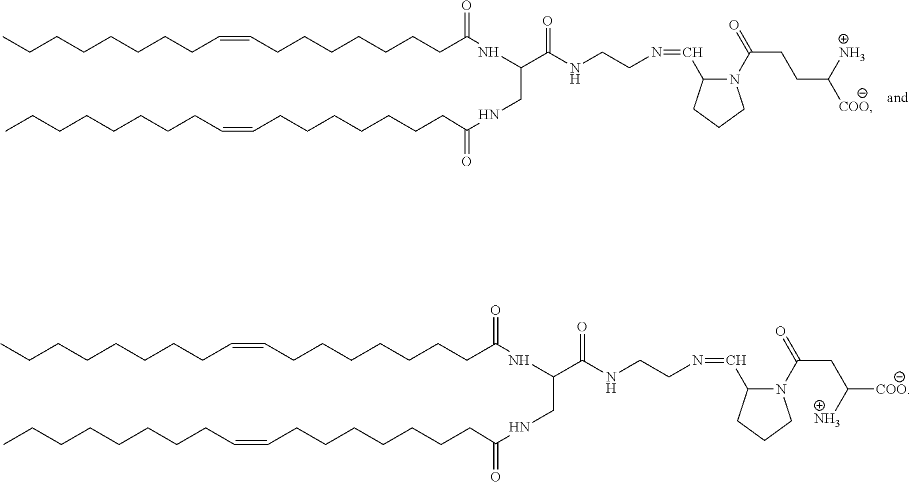

Preferably, the releasable fusogenic lipids of Formula (I) include:

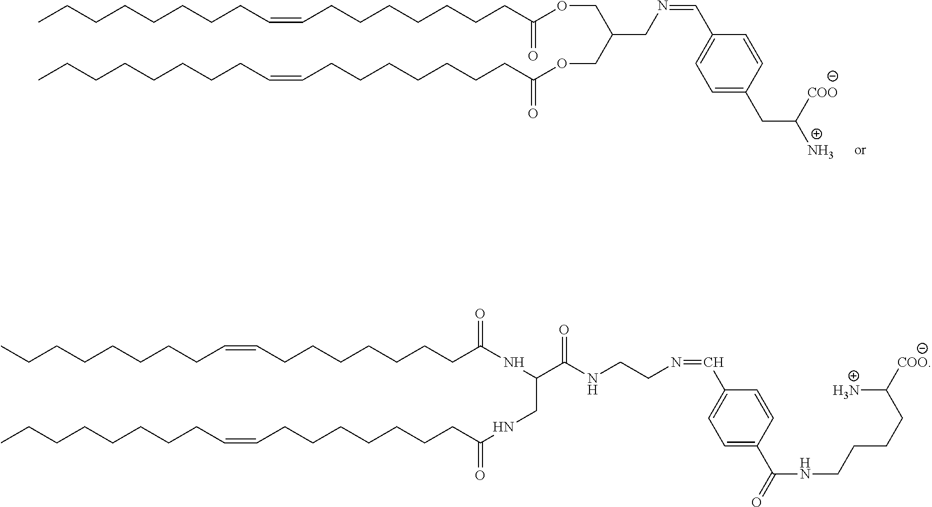

### C. Nanoparticle Composition

### 1. Overview

In one of the present invention, there are provided nanoparticle compositions containing a releasable fusogenic lipid of Formula (I) for the delivery of nucleic acids.

According to the present invention, the nanoparticle composition contains a cationic lipid, a releasable fusogenic lipid of Formula (I), and a PEG lipid.

In one preferred aspect of the invention, the nanoparticle composition includes cholesterol.

In a further aspect of the present invention, the nanoparticle composition described herein may contain art-known fusogenic lipids (non-cationic lipids). The nanoparticle composition containing a mixture of cationic lipids, a mixture of different fusogenic lipids and/or a mixture of different optional PEG lipids are also contemplated.

In another preferred aspect, the nanoparticle composition contains a cationic lipid in a molar ratio ranging from about 10% to about 99.9% of the total lipid present in the nanoparticle composition.

The cationic lipid component can range from about 2% to about 60%, from about 5% to about 50%, from about 10% to about 45%, from about 15% to about 25%, or from about 30% to about 40% of the total lipid present in the nanoparticle composition.

In one preferred embodiment, the cationic lipid is present in amounts from about 15 to about 25% (i.e., 15, 17, 18, 20 or 25%) of the total lipid present in the nanoparticle composition.

According to the present invention, the nanoparticle compositions contain the total fusogenic lipid (preferably releasable fusogenic lipid described herein), including cholesterol and/or noncholesterol-based fusogenic lipid, in a molar ratio of from about 20% to about 85%, from about 25% to about 85%, from about 60% to about 80% (e.g., 65, 75, 78, or 80%) of the total lipid present in the nanoparticle composition. In one preferred embodiment, the total fusogenic/non-cationic lipid is about 80% of the total lipid present in the nanoparticle composition.

In certain embodiments, a noncholesterol-based fusogenic/non-cationic lipid is present in a molar ratio of from about 25 to about 78% (25, 35, 47, 60, or 78%), or from about 60 to about 78% of the total lipid present in the nanoparticle composition. In one embodiment, a noncholesterol-based fusogenic/non-cationic lipid is about 60% of the total lipid present in the nanoparticle composition.

In certain embodiments, the nanopartiele composition includes cholesterol in addition to non-cholesterol fusogenic lipid, in a molar ratio ranging from about 0% to about 60%, from about 10% to about 60%, or from about 20% to about 50% (e.g., 20, 30, 40 or 50%) of the total lipid present in the nanoparticle composition. In one embodiment, cholesterol is about 20% of the total lipid present in the nanoparticle composition.

In certain embodiments, the PEG-lipid contained in the nanoparticle composition ranges in a molar ratio of from about 0.5% to about 20%, from about 1.5% to about 18% of the total lipid present in the nanoparticle composition. In one embodiment of the nanoparticle composition, the PEG lipid is included in a molar ratio of from about 2% to about 10% (e.g., 2, 3, 4, 5, 6, 7, 8, 9 or 10%) of the total lipid. For example, the total PEG lipid is about 2% of the total lipid present in the nanoparticle composition.

For purposes of the present invention, the amount of a releasable fusogenic lipid contained in the nanoparticle composition shall be understood to mean the amount of a releasable fusogenic lipid described herein alone, or the sum of a releasable fusogenic lipid of Formula (I) and any additional art-known fusogenic lipids (either releasable or non-releasable) if present in the nanoparticle composition.

### 2. Releasable Fusogenic Lipids of Formula (I) & Optional Art-Known Fusogenic/Non-Cationic Lipids

According to the present invention, the nanoparticle composition described herein contains a releasable fusogenie of Formula (I). Without being bound by any theory, the releasable fusogenic lipids of Formula (I) facilitate nucleic acids encapsulated in the nanoparticle release from endosomes and the nanoparticle after the nanoparticle enters cells.

In a further aspect of the invention, the nanoparticle composition described herein may include additional art-known fusogenic lipids. Additional suitable art-known fusogenic lipids useful in the nanoparticle composition include neutral fusogenic/noncationic lipids or anionic fusogenic lipids.

Neutral lipids include a lipid that exist either in an uncharged or neutral zwitter ionic form at a selected pH, preferably at physiological pH. Examples of such art-known fusogenic lipids include diacylphosphatidylcholine, diacylphosphatidylethanolamine, ceramide, sphingomyelin, cephalin, cholesterol, cerebrosides and diacylglycerols.

Anionic lipids include a lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and neutral lipids modified with other anionic modifying groups.

Many art-known fusogenic lipids include amphipathic lipids generally having a hydrophobic moiety and a polar head group, and can form vesicles in aqueous solution.

Fusogenic lipids contemplated include naturally-occurring and synthetic phospholipids and related lipids.

A non-limiting list of the non-cationic lipids are selected from among phospholipid and nonphosphous lipid related materials, such as lecithin; lysolecithin; diacylphosphatidylcholine; lysophosphatidylcholine; phosphatidylethanolamine; lysophosphatidylethanolamine; phosphatidylserine; phosphatidylinositol; sphingomyelin; cephalin; ceramide; cardiolipin; phosphatidic acid; phosphatidylglycerol; cerebrosides; dicetylphosphate;

1,2-dilauroyl-sn-glycerol (DLG);

1,2-dimyristoyl-sn-glycerol (DMG);

1,2-dipalmitoyl-sn-glycerol (DPG);

1,2-distearoyl-sn-glycerol (DSG);

1,2-dilauroyl-sn-glycero-3-phosphatidic acid (DLPA);

1,2-dimyristoyl-sn-glycero-3-phosphatidic acid (DMPA);

1,2-dipalmitoyl-sn-glycero-3-phosphatidic acid (DPPA);

1,2-distearoyl-sn-glycero-3-phosphatidic acid (DSPA);

1,2-diarachidoyl-sn-glycero-3-phosphocholine (DAPC);

1,2-dilauroyl-sn-glycero-3-phosphocholine (DLPC);

1,2-dimyristoyl-sn-glycero-3-phosphocholine (DMPC);

1,2-dipalmitoyl-sn-glycero-3-ethylphosphocholine (DPePC);

1,2-dipalmitoyl-sn-glycero-3-phosphocholine or dipalmitoylphosphatidylcholine or dipalmitoylphosphatidylcholine (DPPC);

1,2-distearoyl-sn-glycero-3-phosphocholine or distearoylphosphatidylcholine or distearoylphosphatidylcholine (DSPC);

1,2-dilauroyl-sn-glycero-3-phosphoethanolamine (DLPE);

1,2-dimyristoyl-sn-glycero-3-phosphoethanolamine or dimyristoylphosphoethanolamine (DMPE);

1,2-dipalmitoyl-sn-glycero-3-phosphoethanolamine or dipalmitoylphosphatidylethanolamine (DPPE);

1,2-distearoyl-sn-glycero-3-phosphoethanolamine or distearoylphosphatidylethanolamine (DSPE);

1,2-dilauroyl-sn-glycero-3-phosphoglycerol (DLPG);

1,2-dimyristoyl-sn-glycero-3-phosphoglycerol (DMPG);

1,2-dimyristoyl-sn-glycero-3-phospho-sn-1-glycerol (DMP-sn-1-G);

1,2-dipalmitoyl-sn-glycero-3-phosphoglycerol or dipalmitoylphosphatidylglycerol (DPPG);

1,2-distearoyl-sn-glycero-3-phosphoglycerol (DSPG);

1,2-distearoyl-sn-glycero-3-phospho-sn-1-glycerol (DSP-sn-1-G);

1,2-dipalmitoyl-sn-glycero-3-phospho-L-serine (DPPS);

1-palmitoyl-2-linoleoyl-sn-glycero-3-phosphocholine (PLinoPC);

1-palmitoyl-2-oleoyl-sn-glycero-3-phosphocholine or palmitoyloleoylphosphatidylcholine (POPC);

1-palmitoyl-2-oleoyl-sn-glycero-3-phosphoglycerol (POPG);

1-palmitoyl-2-lyso-sn-glycero-3-phosphocholine (P-lyso-PC);

1-stearoyl-2-lyso-sn-glycero-3-phosphocholine (S-lyso-PC);

1,2-dioleoyl-sn-glycero-3-phosphoethanolamine or dioleoylphosphatidylethanolamine (DOPE);

diphytanoylphosphatidylethanolamine (DPhPE);

1,2-dioleoyl-sn-glycero-3-phosphocholine or dioleoylphosphatidylcholine or dioleoylphosphatidylcholine (DOPC); and

1,2-diphytanoyl-sn-glycero-3-phosphocholine (DPhPC),

dioleoylphosphatidylglycerol (DOPG);

palmitoyloleoylphosphatidylethanolamine (POPE);

dioleoyl-phosphatidylethanolamine 4-(N-maleimidomethyl)-cyclohexane-1-carboxylate (DOPE-mal);

16-O-monomethyl PE;

16-O-dimethyl PE;

18-I-trans PE; 1-stearoyl-2-oleoyl-phosphatidyethanolamine (SOPE);

1,2-dielaidoyl-sn-glycero-3-phophoethanolamine (transDOPE); pharmaceutically acceptable salts and mixtures thereof. Details of the fusogenic lipids are described in US Patent Publication Nos. 2007/0293449 and 2006/0051405.

Noncationic lipids include sterols or steroid alcohols such as cholesterol.

Additional non-cationic lipids are, e.g., stearylamine, dodecylamine, hexadecylamine, acetylpalmitate, glycerolricinoleate, hexadecylstereate, isopropylmyristate, amphoteric acrylic polymers, triethanolaminelauryl sulfate, alkylarylsulfate polyethyloxylated fatty acid amides, and dioctadecyldimethyl ammonium bromide.

Anionic lipids contemplated include phosphatidylserine, phosphatidic acid, phosphatidylcholine, platelet-activation factor (PAF), phosphatidylethanolamine, phosphatidyl-DL-glycerol, phosphatidylinositol, phosphatidylinositol, cardiolipin, lysophosphatides, hydrogenated phospholipids, sphingoplipids, gangliosides, phytosphingosine, sphinganines, pharmaceutically acceptable salts and mixtures thereof.

Suitable noncationic lipids useful for the preparation of the nanoparticle composition described herein include diacylphosphatidylcholine (e.g., distearoylphosphatidylcholine, dioleoylphosphatidylcholine, dipalmitoylphosphatidylcholine and dilinoleoylphosphatidylcholine), diacylphosphatidylethanolamine (e.g., dioleoylphosphatidylethanolamine and palmitoyloleoylphosphatidylethanolamine), ceramide or sphingomyelin. The acyl groups in these lipids are preferably fatty acids having saturated and unsaturated carbon chains such as linoyl, isostearyl, oleyl, elaidyl, petroselinyl, linolenyl, elacostearyl, arachidyl, myristoyl, palmitoyl, and lauroyl. More preferably the acyl groups are lauroyl, myristoyl, palmitoyl, stearoyl or oleoyl, more preferably fatty acids having saturated and unsaturated C₈-C₃₀ (preferably C₁₀-C₂₄) carbon chains.

A variety of phosphatidylcholines useful in the nanoparticle composition described herein includes:

1,2-didecanoyl-sn-glycero-3-phosphocholine (DDPC, C10:0, C10:0);

1,2-dilauroyl-sn-glycero-3-phosphocholine (DLPC, C12:0, C12:0);

1,2-dimyristoyl-sn-glycero-3-phosphocholine (DMPC, C14:0, C14:0);

1,2-dipalmitoyl-sn-glycero-3-phosphocholine (DPPC, C16:0, C16:0);

1,2-distearoyl-sn-glycero-3-phosphocholine (DSPC, C18:0, C18:0);

1,2-dioleoyl-sn-glycero-3-phosphocholine (DOPC, C18:1, C18:1);

1,2-dierucoyl-sn-glycero-3-phosphocholine (DEPC, C22:1, C22:1);

1,2-dieicosapentaenoyl-sn-glycero-3-phosphocholine (EPA-PC, C20:5, C20:5);

1,2-didocosahexaenoyl-sn-glycero-3-phosphocholine (DHA-PC, C22:6, C22:6);

1-myristoyl-2-palmitoyl-sn-glycero-3-phosphocholine (MPPC, C14:0, C16:0);

1-myristoyl-2-stearoyl-sn-glycero-3-phosphocholine (MSPC, C14:0, C18:0);

1-palmitoyl-2-stearoyl-sn-glycero-3-phosphocholine (PMPC, C16:0, C14:0);

1-palmitoyl-2-stearoyl-sn-glycero-3-phosphocholine (PSPC, C16:0, C18:0);

1-stearoyl-2-myristoyl-sn-glycero-3-phosphocholine (SMPC, C18:0, C14:0);

1-stearoyl-2-palmitoyl-sn-glycero-3-phosphocholine (SPPC, C18:0, C16:0);

1,2-myristoyl-oleoyl-sn-glycero-3-phosphoethanolamine (MOPC, C14:0, C18:0);

1,2-palmitoyl-oleoyl-sn-glycero-3-phosphoethanolamine (POPC, C16:0, C18:1);

1,2-stearoyl-oleoyl-sn-glycero-3-phosphoethanolamine (POPC, C18:0, C18:1), pharmaceutically acceptable salts and mixtures thereof.

A variety of lysophosphatidylcholine useful in the nanoparticle composition described herein includes:

1-myristoyl-2-lyso-sn-glycero-3-phosphocholine (M-LysoPC, C14:0);

1-malmitoyl-2-lyso-sn-glycero-3-phosphocholine (P-LysoPC, C16:0);

1-stearoyl-2-lyso-sn-glycero-3-phosphocholine (S-LysoPC, C18:0), pharmaceutically acceptable salts and mixtures thereof.

A variety of phosphatidylglycerols useful in the nanoparticle composition described herein are selected from among:

hydrogenated soybean phosphatidylglycerol (HSPG);

non-hydrogenated egg phosphatidylgycerol (EPG);

1,2-dimyristoyl-sn-glycero-3-phosphoglycerol (DMPG, C14:0, C14:0);

1,2-dipalmitoyl-sn-glycero-3-phosphoglycerol (DPPG, C16:0, C16:0);

1,2-distearoyl-sn-glycero-3-phosphoglycerol (DSPG, C18:0, C18:0);

1,2-dioleoyl-sn-glycero-3-phosphoglycerol (DOPG, C18:1, C18:1);

1,2-dierucoyl-sn-glycero-3-phosphoglycerol (DEPG, C22:1, C22:1);

1-palmitoyl-2-oleoyl-sn-glycero-3-phosphoglycerol (POPG, C16:0, C18:1), pharmaceutically acceptable salts and mixtures thereof.

A variety of phosphatidic acids useful in the nanoparticle composition described herein includes:

1,2-dimyristoyl-sn-glycero-3-phosphatidic acid (DMPA, C14:0, C14:0);

1,2-dipalmitoyl-sn-glycero-3-phosphatidic acid (DPPA, C16:0, C16:0);

1,2-distearoyl-sn-glycero-3-phosphatidic acid (DSPA, C18:0, C18:0), pharmaceutically acceptable salts and mixtures thereof.

A variety of phosphatidylethanolamines useful in the nanoparticle composition described herein includes:

hydrogenated soybean phosphatidylethanolamine (HSPE);

non-hydrogenated egg phosphatidylethanolamine (EPE);

1,2-dimyristoyl-sn-glycero-3-phosphoethanolamine (DMPE, C14:0, C14:0);

1,2-dipalmitoyl-sn-glycero-3-phosphoethanolamine (DPPE, C16:0, C16:0);

1,2-distearoyl-sn-glycero-3-phosphoethanolamine (DSPE, C18:0, C18:0);

1,2-dioleoyl-sn-glycero-3-phosphoethanolamine (DOPE, C18:1, C18:1);

1,2-dioleoyl-sn-glycero-3-phosphoethanolamine (DEPE, C22:1, C22:1);

1,2-dierucoyl-sn-glycero-3-phosphoethanolamine (POPE, C16:0, C18:1), pharmaceutically acceptable salts and mixtures thereof.

A variety of phosphatidylserines useful in the nanoparticle composition described herein includes:

1,2-dimyristoyl-sn-glycero-3-phospho-L-serine (DMPS, C14:0, C14:0);

1,2-dipalmitoyl-sn-glycero-3-phospho-L-serine (DPPS, C16:0, C16:0);

1,2-distearoyl-sn-glycero-3-phospho-L-serine (DSPS, C18:0, C18:0);

1,2-dioleoyl-sn-glycero-3-phospho-L-serine (DOPS, C18:1, C18:1);

1-palmitoyl-2-oleoyl-sn-3-phospho-L-serine (POPS, C16:0, C18:1), pharmaceutically acceptable salts and mixtures thereof.

In one preferred embodiment, suitable neutral lipids useful for the preparation of the nanoparticle composition described herein include, for example,

dioleoylphosphatidylethanolamine (DOPE),

distearoylphosphatidylethanolamine (DSPE),

palmitoyloleoylphosphatidylethanolamine (POPE),

egg phosphatidylcholine (EPC),

dipalmitoylphosphatidylcholine (DPPC),

distearoylphosphatidylcholine (DSPC),

dioleoylphosphatidylcholine (DOPC),

palmitoyloleoylphosphatidylcholine (POPC),

dipalmitoylphosphatidylglycerol (DPPG),

dioleoylphosphatidylglycerol (DOPG),

dioleoyl-phosphatidylethanolamine 4-(N-maleimidomethyl)-cyclohexane-1-carboxylate (DOPE-mal), cholesterol, pharmaceutically acceptable salts and mixtures thereof.

In certain preferred embodiments, the nanoparticle composition described herein includes DSPC, EPC, DOPE, etc, and mixtures thereof.

In a further aspect of the invention, the nanoparticle composition contains non-cationic lipids such as sterol. The nanoparticle composition preferably contains cholesterol or analogs thereof, and more preferably cholesterol.

### 3. Cationic Lipids

According to the present invention, the nanoparticle composition described herein can include a cationic lipid. Suitable lipids contemplated include, for example:

N-[1-(2,3-dioleoyloxy)propyl]-N,N,N-trimethylammonium chloride (DOTMA);

1,2-bis(oleoyloxy)-3-3-(trimethylammonium)propane or N-(2,3-dioleoyloxy)propyl)-N,N,N-trimethylammonium chloride (DOTAP);

1,2-bis(dimyrstoyloxy)-3-3-(trimethylammonia)propane (DMTAP);

1,2-dimyristyloxypropyl-3-dimethylhydroxyethylammonium bromide or N-(1,2-dimyristyloxyprop-3-yl)-N,N-dimethyl-N-hydroxyethyl ammonium bromide (DMRIE);

dimethyldioctadecylammonium bromide or N,N-distearyl-N,N-dimethylammonium bromide (DDAB);

3-(N-(N′,N′-dimethylaminoethane)carbamoyl)cholesterol (DC-Cholesterol);

3β-[N′,N′-diguanidinoethyl-aminoethane)carbamoyl cholesterol (BGTC);

2-(2-(3-(bis(3-aminopropyl)amino)propylamino)acetamido)-N,N-ditetradecylacetamide (RPR209120);

1,2-dialkenoyl-sn-glycero-3-ethylphosphocholines (i.e., 1,2-dioleoyl-sn-glycero-3-ethylphosphocholine, 1,2-distearoyl-sn-glycero-3-ethylphosphocholine and 1,2-dipalmitoyl-sn-glycero-3-ethylphosphocholine);

tetramethyltetrapalmitoyl spermine (TMTPS);

tetramethyltetraoleyl spermine (TMTOS);

tetramethlytetralauryl spermine (TMTLS);

tetramethyltetramyristyl spermine (TMTMS);

tetramethyldioleyl spermine (TMDOS);

2,5-bis(3-aminopropylamino)-N-(2-(dioctadecylamino)-2-oxoethyl)pentanamide (DOGS);

2,5-bis(3-aminopropylamino)-N-(2-(di(Z)-octadeca-9-dienylamino)-2-oxoethy-1)pentanamide (DOGS-9-en);

2,5-bis(3-aminopropylamino)-N-(2-(di(9Z,12Z)-octadeca-9,12-dienylamino)-2-oxoethyl)pentanamide (DLinGS);

N4-Spermine cholesteryl carbamate (GL-67);

(9Z,9′Z)-2-(2,5-bis(3-aminopropylamino)pentanamido)propane-1,3-diyl-dioctadec-9-enoate (DOSPER);

2,3-dioleyloxy-N-[2 (sperminecarboxamido)ethyl]-N,N-dimethyl-1-propanaminium trifluoroacetate (DOSPA);

1,2-dimyristoyl-3-trimethylammonium-propane; 1,2-distearoyl-3-trimethylammonium-propane;

dioctadecyldimethylammonium (DODMA);

distearyldimethylammonium (DSDMA);

N,N-dioleyl-N,N-dimethylammonium chloride (DODAC); pharmaceutically acceptable salts and mixtures thereof.

Details of cationic lipids are also described in US2007/0293449 and U.S. Pat. Nos. 4,897,355; 5,279,833; 6,733,777; 6,376,248; 5,736,392; 5,686,958; 5,334,761; 5,459,127; 2005/0064595; U.S. Pat. Nos. 5,208,036; 5,264,618; 5,279,833; 5,283,185; 5,753,613; and 5,785,992.

In one preferred aspect, the cationic lipids would carry a net positive charge at a selected pH, such as pH<13 (e.g. pH 6-12, pH 6-8). One preferred embodiment of the nanoparticle compositions includes the cationic lipids described herein having the structure:

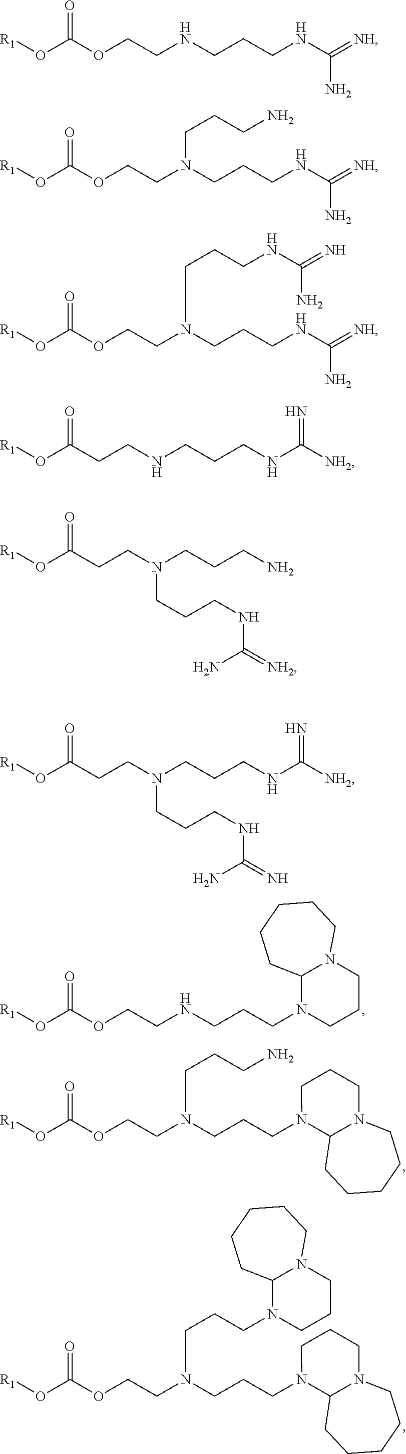

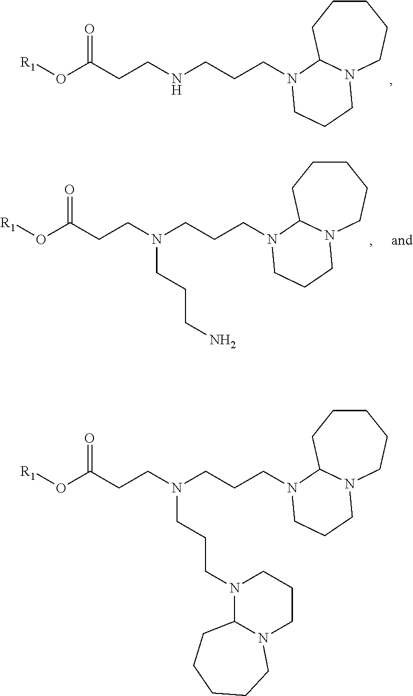

wherein R₁ is cholesterol or an analog thereof.

More preferably, a nanoparticle composition includes the cationic lipid having the structure:

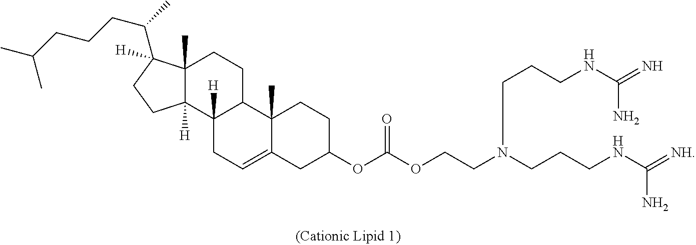

Details of cationic lipids are also described in PCT/US09/52396, the contents of which are incorporated herein by reference.

Additionally, commercially available preparations including cationic lipids can be used: for example, LIPOFECTIN® (cationic liposomes containing DOTMA and DOPE, from GIBCO/BRL, Grand Island, N.Y., LISA); LIPOFECTAMINE® (cationic liposomes containing DOSPA and DOPE, from GIBCO/BRL, Grand Island, N.Y., USA); and TRANSFECTAM® (cationic liposomes containing DOGS from Promega Corp., Madison, Wis., USA).

### 4. PEG Lipids

According to the present invention, the nanoparticle composition described herein contains a PEG lipid. The PEG lipids extend circulation of the nanoparticle described herein and prevent the premature excretion of the nanoparticles from the body. The PEG lipids reduce the immunogenicity and enhance the stability of the nanoparticles.

The PEG lipids useful in the nanoparticle compositions include PEGylated forms of fusogenic/noncationic lipids. The PEG lipids include, for example, PEG conjugated to diacylglycerol (PEG-DAG), PEG conjugated to diacylglycamides, PEG conjugated to dialkyloxypropyls (PEG-DAA), PEG conjugated to phospholipids such as PEG coupled to phosphatidylethanolamine (PEG-PE), PEG conjugated to ceramides (PEG-Cer), PEG conjugated to cholesterol derivatives (PEG-Chol) or mixtures thereof. See U.S. Pat. Nos. 5,885,613 and 5,820,873, and US Patent Publication No. 2006/051405, the contents of each of which are incorporated herein by reference.

PEG is generally represented by the structure:

—O—(CH₂CH₂O)ₙ—

where (n) is a positive integer from about 5 to about 2300, preferably from about 5 to about 460 so that the polymeric portion of PEG lipid has an average number molecular weight of from about 200 to about 100,000 daltons, preferably from about 200 to about 20,000 daltons. (n) represents the degree of polymerization for the polymer, and is dependent on the molecular weight of the polymer.

In one preferred aspect, the PEG is a polyethylene glycol with a number average molecular weight ranging from about 200 to about 20,000 daltons, more preferably from about 500 to about 10,000 daltons, yet more preferably from about 1,000 to about 5,000 daltons (i.e., about 1,500 to about 3,000 daltons). In one embodiment, the PEG has a molecular weight of about 2,000 daltons. In another embodiment, the PEG has a molecular weight of about 750 daltons.

Alternatively, the polyethylene glycol (PEG) residue portion can be represented by the structure:

—Y₇₁—(CH₂CH₂O)ₙ—CH₂CH₂Y₇₁—,

—Y₇₁—(CH₂CH₂O)ₙ—CH₂C(═Y₇₂)—Y₇₁—,

—Y₇₁—C(═Y₇₂)—(CH₂)ₐ₁₂—Y₇₃—(CH₂CH₂O)ₙ—CH₂CH₂—Y₇₃—(CH₂)ₐ₁₂—C(═Y₇₂)—Y₇₁— and

—Y₇₁—(CR₇₁R₇₂)ₐ₁₂—Y₇₃—(CH₂)_{b12}—O—(CH₂CH₂O)ₙ—(CH_{b12}—Y₇₃—(CR₇₁R₇₂)ₐ₁₂—Y₇₁—,

wherein:

Y₇₁ and Y₇₃ are independently O, S, SO, SO₂, NR₇₃ or a bond;

Y₇₂ is O, S, or NR₇₄, preferably oxygen;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy, preferably hydrogen, methyl, ethyl or propyl;

(a12) and (b12) are independently zero or positive integers, preferably zero or an integer from about 1 to about 6 (i.e., 1, 2, 3, 4, 5, 6), and more preferably 1 or 2; and

(n) is an integer from about 5 to about 2300, preferably from about 5 to about 460.

The terminal end of PEG can end with H, NH₂, OH, CO₂H, C₁₋₆ alkyl (e.g., methyl, ethyl, propyl), C₁₋₆ alkoxy, acyl or aryl. In one preferred embodiment, the terminal hydroxyl group of PEG is substituted with a methoxy or methyl group. In one preferred embodiment, the PEG employed in the PEG lipid is methoxy PEG.

The PEG may be directly conjugated to lipids or via a linker moiety. The polymers for conjugation to a lipid structure are converted into a suitably activated polymer, using the activation techniques described in U.S. Pat. Nos. 5,122,614 and 5,808,096 and other techniques known in the art without undue experimentation.

Examples of activated PEGs useful for the preparation of a PEG lipid include, for example, methoxypolyethylene glycol-succinate, mPEG-NHS, methoxypolyethylene succinimidyl succinate, methoxypolyethyleneglycol-acetic acid (mPEG-CH₂COOH), methoxypolyethylene glycol-amine (mPEG-NH₂), and methoxypolyethylene glycol-tresylate (mPEG-TRES).

In certain aspects, polymers having terminal carboxylic acid groups can be used for the preparation of the PEG lipids. Methods of preparing polymers having terminal carboxylic acids in high purity are described in U.S. patent application Ser. No. 11/328,662, the contents of which are incorporated herein by reference.

In alternative aspects, polymers having terminal amine groups can be employed to make the PEG-lipids. The methods of preparing polymers containing terminal amines in high purity are described in U.S. patent application Ser. Nos. 11/508,507 and 11/537,172, the contents of each of which are incorporated by reference.

PEG and lipids can be bound via a linkage, i.e. a non-ester containing linker moiety or an ester containing linker moiety. Suitable non-ester containing linkers include, but are not limited to, an amido linker moiety, an amino linker moiety, a carbonyl linker moiety, a carbamate linker moiety, a carbonate (OC(═O)O) linker moiety, a urea linker moiety, an ether linker moiety, a succinyl linker moiety, and combinations thereof. Suitable ester linker moieties include, e.g., succinoyl, phosphate esters (—O—P(═O)(OH)—O—), sulfonate esters, and combinations thereof.

In one embodiment, the nanoparticle composition described herein can include a polyethyleneglycol-diacylglycerol (PEG-DAG) or polyethylene-diacylglycamide. Suitable polyethyleneglycol-diacylglycerol or polyethyleneglycol-diacylglycamide conjugates include a dialkylglycerol or dialkylglycamide group having alkyl chain length independently containing from about C₄ to about C₃₀ (preferably from about C₈ to about C₂₄) saturated or unsaturated carbon atoms. The dialkylglycerol or dialkylglycamide group can further include one or more substituted alkyl groups.

The term “diacylglycerol” (DAG) used herein refers to a compound having two fatty acyl chains, R₁₁₁ and R₁₁₂. The R₁₁₁ and R₁₁₂ have the same or different carbon chain in length of about 4 to about 30 carbons (preferably about 8 to about 24) and are bonded to glycerol by ester linkages. The acyl groups can be saturated or unsaturated with various degrees of unsaturation. DAG has the general formula:

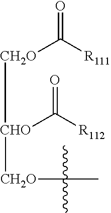

In one preferred embodiment, the PEG-diacylglycerol conjugate is a PEG-dilaurylglycerol (C12), a PEG-dimyristylglycerol (C14, DMG), a PEG-dipalmitoylglycerol (C16, DPG) or a PEG-distearylglycerol (C18, DSG). Those of skill in the art will readily appreciate that other diacylglycerols are also contemplated in the PEG-diacylglycol conjugate. Suitable PEG-diacylglycerol conjugates for use in the present invention, and methods of making and using them, are described in U.S. Patent Publication No. 2003/0077829, and PCT Patent Application No. CA 02/00669, the contents of each of which are incorporated herein by reference.

Examples of the PEG-diacylglycerol conjugate can be selected from among PEG-dilaurylglycerol (C12), PEG-dimyristylglycerol (C14), PEG-dipalmitoylglycerol (C16), PEG-disterylglycerol (C18). Examples of the PEG-diacylglycamide conjugate includes PEG-dilaurylglycamide (C12), PEG-dimyristylglycamide (C14), PEG-dipalmitoyl-glycamide (C16), and PEG-disterylglycamide (C18).

In another embodiment, the nanoparticle composition described herein can include a polyethyleneglycol-dialkyloxypropyl conjugates (PEG-DAA).

The term “dialkyloxypropyl” refers to a compound having two alkyl chains, R₁₁₁ and R₁₁₂. The R₁₁₁ and R₁₁₂ alkyl groups include the same or different carbon chain length between about 4 to about 30 carbons (preferably about 8 to about 24). The alkyl groups can be saturated or have varying degrees of unsaturation. Dialkyloxypropyls have the general formula:

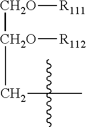

wherein R₁₁₁ and R₁₁₂ alkyl groups are the same or different alkyl groups having from about 4 to about 30 carbons (preferably about 8 to about 24). The alkyl groups can be saturated or unsaturated. Suitable alkyl groups include, but are not limited to, lauryl (C12), myristyl (C14), palmityl (C16), stearyl (C18), oleoyl (C18) and icosyl (C20).

In one embodiment, R₁₁₁ and R₁₁₂ are both the same, i.e., R₁₁₁ and R₁₁₂ are both myristyl (C14), both stearyl (C18) or both oleoyl (C18), etc. In another embodiment, R₁₁₁ and R₁₁₂ are different, i.e., R₁₁₁ is myristyl (C14) and R₁₁₂ is stearyl (C18). In a preferred embodiment, the PEG-dialkylpropyl conjugates include the same R₁₁₁ and R₁₁₂.

In yet another embodiment, the nanoparticle composition described herein can include PEG conjugated to phosphatidylethanolamines (PEG-PE). The phosphatidylethanolamines useful for the PEG lipid conjugation can contain saturated or unsaturated fatty acids with carbon chain lengths in the range of about 4 to about 30 carbons (preferably about 8 to about 24). Suitable phosphatidylethanolamines include, but are not limited to dimyristoylphosphatidylethanolamine (DMPE), dipalmitoylphosphatidylethanolamine (DPPE), dioleoylphosphatidylethanolamine (DOPE) and distearoylphosphatidylethanolamine (DSPE).

In yet another embodiment, the nanoparticle composition described herein can include PEG conjugated to ceramides (PEG-Cer). Ceramides have only one acyl group. Ceramides can have saturated or unsaturated fatty acids with carbon chain lengths in the range of about 4 to about 30 carbons (preferably about 8 to about 24).

In alternative embodiments, the nanoparticle composition described herein can include PEG conjugated to cholesterol derivatives. The term “cholesterol derivative” means any cholesterol analog containing a cholesterol structure with modification, i.e., substitutions and/or deletions thereof. The term cholesterol derivative herein also includes steroid hormones and bile acids.

Illustrative examples of PEG lipids include N-(carbonyl-methoxypolyethyleneglycol)-1,2-dimyristoyl-sn-glycero-3-phosphoethanolamine (^{2 kDa}mPEG-DMPE or ^{51 kDa}mPEG-DMPE); N-(carbonyl-methoxypolyethyleneglycol)-1,2-dipalmitoyl-sn-glycero-3-phosphoethanolamine (^{2 kDa}mPEG-DPPE or ^{5 kDa}mPEG-DPPE); N-(carbonyl-methoxypolyethyleneglycol)-1,2-distearoyl-sn-glycero-3-phosphoethanolamine (^{750 Da}mPEG-DSPE, ^{2 kDa}mPEG-DSPE, ^{5 kDa}mPEG-DSPE); and pharmaceutically acceptable salts thereof (i.e., sodium salt) and mixtures thereof.

In certain preferred embodiments, the nanoparticle composition described herein includes a PEG lipid having PEG-DAG or PEG-ceramide, wherein PEG has molecular weight from about 200 to about 20,000, preferably from about 500 to about 10,000, and more preferably from about 1,000 to about 5,000.

A few illustrative embodiments of PEG-DAG and PEG-ceramide are provided in Table 1.

TABLE 1
PEG-Lipid

| | |
|---|---|
| PEG-DAG | mPEG-diimyristoylglycerol |
| | mPEG-dipalmitoylglycerol |
| | mPEG-distearoylglycerol |
| PEG-Ceramide | mPEG-CerC8 |
| | mPEG-CerC14 |
| | mPEG-CerC16 |
| | mPEG-CerC20 |

Preferably, the nanoparticle composition described herein includes the PEG lipid selected from among PEG-DSPE, PEG-dipalmitoylglycamide (C16), PEG-Ceramide (C16), etc. and mixtures thereof. The structures of mPEG-DSPE, mPEG-dipalmitoylglycamide (C16), and mPEG-Ceramide (C16) are as follows:

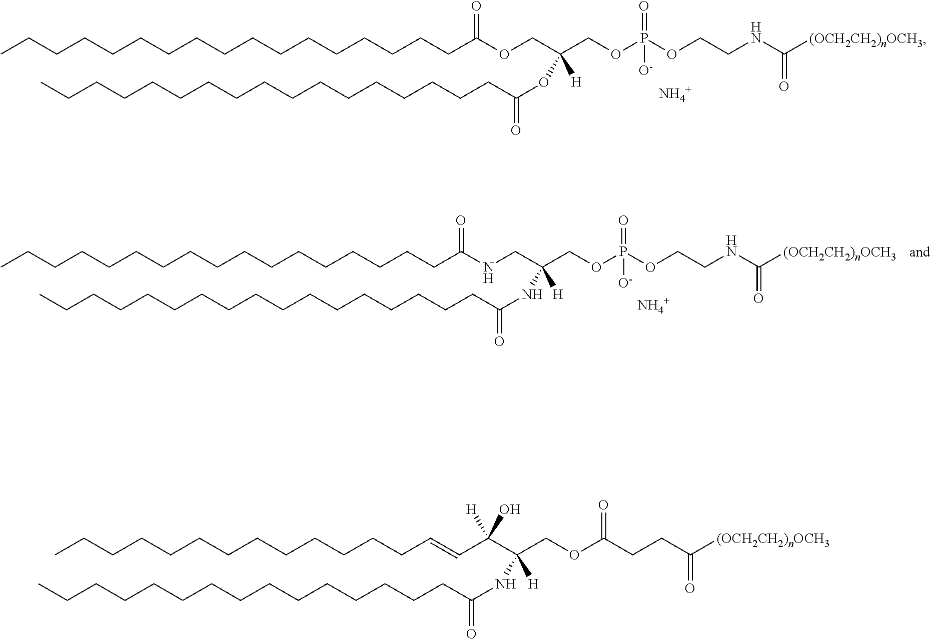

wherein, (n) is an integer from about 5 to about 2300, preferably from about 5 to about 460.

In one preferred embodiment, (n) is about 45.

In a further embodiment and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation can be employed as described herein as for PAOs such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

In yet a further embodiment, the nanoparticle described herein can include PEG lipids with a releasable linker such as ketal or imine. Such releasable PEG lipids allow nucleic acids (oligonucleotides) to dissociate from the delivery system after the delivery system enters the cells. Additional details of such releasable PEG lipids are also described in U.S. Provisional Patent Application Nos. 61/115,379 and 61/115,371, entitled “Releasable Polymeric Lipids Based on Imine Moiety For Nucleic Acids Delivery System” and “Releasable Polymeric Lipids Based on Ketal or Acetal Moiety For Nucleic Acids Delivery System” respectively, and PCT Patent Application No. ______, filed on even date, and entitled “Releasable Polymeric Lipids For Nucleic Acids Delivery Systems”, the contents of each of which are incorporated herein by reference.

### 5. Nucleic Acids/Oligonucleotides

The nanoparticle compositions described herein can be used for delivering various nucleic acids into cells or tissues. The nucleic acids include plasmids and oligonucleotides. Preferably, the nanoparticle compositions described herein are used for delivery of oligonucleotides.

In order to more fully appreciate the scope of the present invention, the following terms are defined. The artisan will appreciate that the terms, “nucleic acid” or “nucleotide” apply to deoxyribonucleic acid (“DNA”), ribonucleic acid, (“RNA”) whether single-stranded or double-stranded, unless otherwise specified, and to any chemical modifications or analogs thereof, such as, locked nucleic acids (LNA). The artisan will readily understand that by the term “nucleic acid,” included are polynucleic acids, derivates, modifications and analogs thereof. An “oligonucleotide” is generally a relatively short polynucleotide, e.g., ranging in size from about 2 to about 200 nucleotides, preferably from about 8 to about 50 nucleotides, more preferably from about 8 to about 30 nucleotides, and yet more preferably from about 8 to about 20 or from about 15 to about 28 in length. The oligonucleotides according to the invention are generally synthetic nucleic acids, and are single stranded, unless otherwise specified. The terms, “polynucleotide” and “polynucleic acid” may also be used synonymously herein.

The oligonucleotides (analogs) are not limited to a single species of oligonucleotide but, instead, are designed to work with a wide variety of such moieties, it being understood that linkers can attach to one or more of the 3′- or 5′-terminals, usually PO₄ or SO₄ groups of a nucleotide. The nucleic acid molecules contemplated can include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogues such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18th & 19th Nov. 2003, Hamburg, Germany, the contents of which are incorporated herein by reference.

Modifications to the oligonucleotides contemplated by the invention include, for example, the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5′ cap structure

For purposes of the present invention, “cap structure” shall be understood to mean chemical modifications, which have been incorporated at either terminus of the oligonucleotide. The cap can be present at the 5′-terminus (5′-cap) or at the 3′-terminus (3′-cap) or can be present on both termini. A non-limiting example of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide; 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. Details are described in WO 97/26270, the contents of which are incorporated by reference herein. The 3′-cap can include for example 4′,5′-methylene nucleotide; 1-(beta-D-erythrofuranosyl) nucleotide; 4′-thio nucleotide, carbocyclic nucleotide; 5′-aminoalkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide;5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer, 1993, Tetrahedron 49, 1925; the contents of which are incorporated by reference herein.

A non-limiting list of nucleoside analogs have the structure:

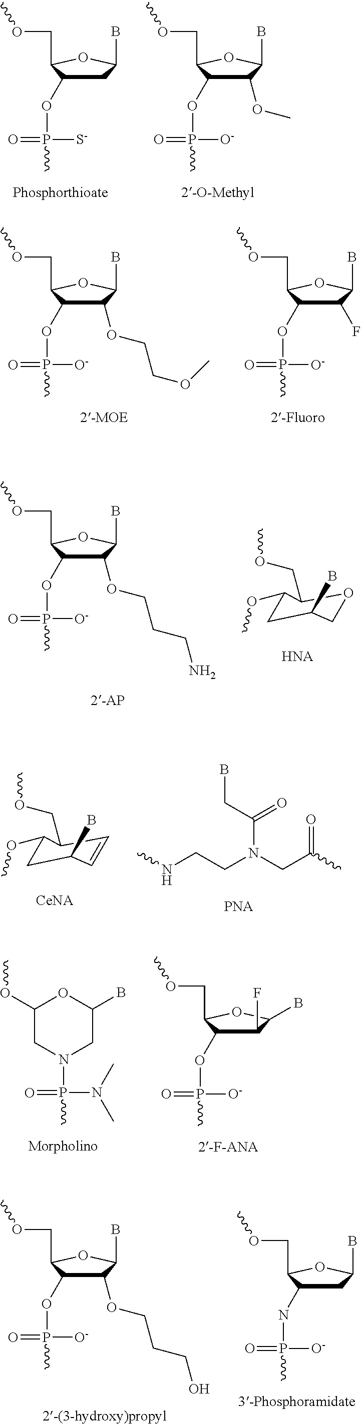

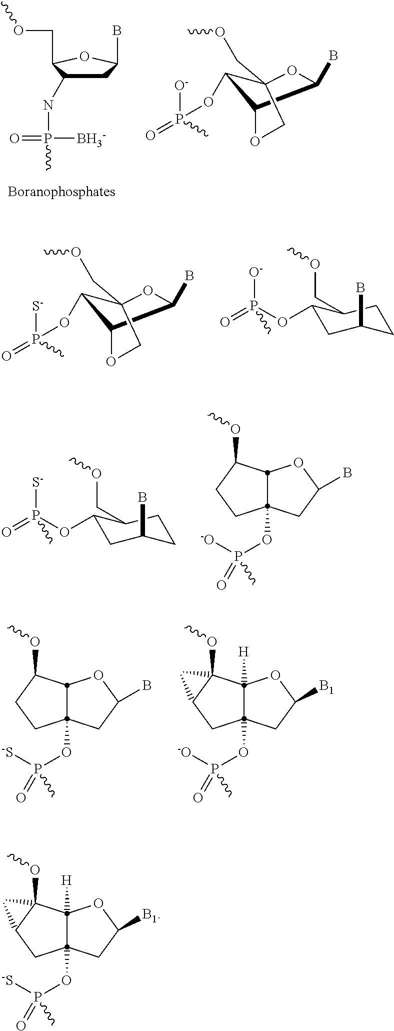

See more examples of nucleoside analogues described in Freier & Altmann; Nucl. Acid Res., 1997, 25, 4429-4443 and Uhlmann; Curr. Opinion in Drug Development, 2000, 3(2), 293-213, the contents of each of which are incorporated herein by reference.

The term “antisense,” as used herein, refers to nucleotide sequences which are complementary to a specific DNA or RNA sequence that encodes a gene product or that encodes a control sequence. The term “antisense strand” is used in reference to a nucleic acid strand that is complementary to the “sense” strand. In the normal operation of cellular metabolism, the sense strand of a DNA molecule is the strand that encodes polypeptides and/or other gene products. The sense strand serves as a template for synthesis of a messenger RNA (“mRNA”) transcript (an antisense strand) which, in turn, directs synthesis of any encoded gene product. Antisense nucleic acid molecules may be produced by any art-known methods, including synthesis. Once introduced into a cell, this transcribed strand combines with natural sequences produced by the cell to form duplexes. These duplexes then block either the further transcription of the mRNA or its translation. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another nucleic acid sequence. A percent complementarity indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 90%, and 100% complementary. “Perfectly complementary” means that all the contiguous residues of a nucleic acid sequence form hydrogen bonds with the same number of contiguous residues in a second nucleic acid sequence.

The nucleic acids (such as one or more same or different oligonucleotides or oligonucloetide derivatives) useful in the nanoparticle described herein can include from about 5 to about 1000 nucleic acids, and preferably relatively short polynucleotides, e.g., ranging in size preferably from about 8 to about 50 nucleotides in length (e.g., about 8, 9, 10, 11, 12, 13, 14, 15, 16, 17, 18, 19, 20, 21, 22, 23, 24, 25, 26, 27, 28, 29 or 30).

In one aspect, useful nucleic acids encapsulated within the nanoparticle described herein include oligonucleotides and oligodeoxynucleotides with natural phosphorodiester backbone or phosphorothioate backbone or any other modified backbone analogues such as:

LNA (Locked Nucleic Acid);

PNA (nucleic acid with peptide backbone);

short interfering RNA (siRNA);

microRNA (miRNA);

nucleic acid with peptide backbone (PNA);

phosphorodiamidate morpholino oligonucleotides (PMO);

tricyclo-DNA;

decoy ODN (double stranded oligonucleotide);

catalytic RNA sequence (RNAi);

ribozymes;

aptamers;

spiegelmers (L-conformational oligonucleotides);

CpG oligomers, and the like, such as those disclosed at:

Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

In another aspect of the nucleic acids encapsulated within the nanoparticle, oligonucleotides can optionally include any suitable art-known nucleotide analogs and derivatives, including those listed by Table 2, below:

TABLE 2
Representative Nucleotide Analogs And Derivatives

| | |
|---|---|
| 4-acetylcytidine | 5-methoxyaminomethyl-2-thiouridine |
| 5-(carboxyhydroxymethyl)- | beta, D-mannosylqueuosine |
| uridine | |
| 2′-O-methylcytidine | 5-methoxycarbonylmethyl-2-thiouridine |
| 5-methoxycarbonylmethyl- | 5-carboxymethylaminomethyl-2-thio- |
| uridine | uridine |
| 5-methoxyuridine | 5-carboxymethylaminomethyluridine |
| Dihydrouridine | 2-methylthio-N6-isopentenyladenosine |
| 2′-O-methylpseudouridine | N-[(9-beta-D-ribofuranosyl-2-methylthio- |
| | purine-6-yl)carbamoyl]threonine |
| D-galactosylqueuosine | N-[(9-beta-D-ribofuranosylpurine-6-yl)N- |
| | methylcarbamoyl]threonine |
| 2′-O-methylguanosine | uridine-5-oxyacetic acid-methylester |
| 2′-halo-adenosine | 2′-halo-cytidine |
| 2′-halo-guanosine | 2′-halo-thymine |
| 2′-halo-uridine | 2′-halo-methylcytidine |
| 2′-amino-adenosine | 2′-amino-cytidine |
| 2′-amino-guanosine | 2′-amino-thymine |
| 2′-amino-uridine | 2′-amino-methylcytidine |
| Inosine | uridine-5-oxyacetic acid |
| N6-isopentenyladenosine | Wybutoxosine |
| 1-methyladensine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-[(9-beta-D-ribofuranosylpurine-6-yl)- |
| | carbamoyl]threonine |
| 5-methylcytidine | 2′-O-methyl-5-methyluridine |
| N6-methyladenosine | 2′-O-methyluridine |
| 7-methylguanosine | Wybutosine |
| 5-methylaminomethyl- | 3-(3-amino-3-carboxy-propyl)uridine |
| uridine | |
| Locked-adenosine | Locked-cytidine |
| Locked-guanosine | Locked-thymine |
| Locked-uridine | Locked-methylcytidine |

In one preferred aspect, the target oligonucleotides encapsulated in the nanoparticles include, for example, but are not limited to, oncogenes, pro-angiogenesis pathway genes, pro-cell proliferation pathway genes, viral infectious agent genes, and pro-inflammatory pathway genes.

In one preferred embodiment, the oligonucleotide encapsulated within the nanoparticle described herein is involved in targeting tumor cells or downregulating a gene or protein expression associated with tumor cells and/or the resistance of tumor cells to anticancer therapeutics. For example, antisense oligonucleotides for downregulating any art-known cellular proteins associated with cancer, e.g., BCL-2 can be used for the present invention. See U.S. patent application Ser. No. 10/822,205 filed Apr. 9, 2004, the contents of which are incorporated by reference herein. A non-limiting list of preferred therapeutic oligonucleotides includes antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and antisense beta-catenin oligonucleotides.

More preferably, the oligonucleotides according to the invention described herein include phosphorothioate backbone and LNA.

In one preferred embodiment, the oligonucleotide can be, for example, antisense survivin LNA, antisense ErbB3 LNA, or antisense HIF1-α LNA.

In another preferred embodiment, the oligonucleotide can be, for example, an oligonucleotide that has the same or substantially similar nucleotide sequence as does Genasense® (a/k/a oblimersen sodium, produced by Genta Inc., Berkeley Heights, N.J.). Genasense® is an 18-mer phosphorothioate antisense oligonucleotide (SEQ ID NO: 4), that is complementary to the first six codons of the initiating sequence of the human bcl-2 mRNA (human bcl-2 mRNA is art-known, and is described, e.g., as SEQ ID NO: 19 in U.S. Pat. No. 6,414,134, incorporated by reference herein).

Preferred embodiments contemplated include:

(i) antisense Survivin LNA oligomer (SEQ ID NO: 1)
^{m}Cₛ-Tₛ-^{m}Cₛ-Aₛ-aₛ-tₛ-cₛ-cₛ-aₛ-tₛ-gₛ-gₛ-^{m}Cₛ-Aₛ-Gₛ-c;
where the upper case letter represents LNA, the “s” represents a phosphorothioate backbone;

(ii) antisense Bcl2 siRNA:
SENSE 5′-gcaugcggccucuguuugadTdT-3′ (SEQ ID NO: 2)
ANTISENSE 3′-dTdTcguacgccggagacaaacu-5′(SEQ ID NO: 3)
where dT represents DNA;

(iii) Genasense (phosphorothioate antisense oligonucleotide): (SEQ ID NO: 4)
tₛ-cₛtₛ-cₛ₋cₛ-cₛ-aₛ-gₛ-cₛ-gₛ-tₛ-gₛ-cₛ-gₛ-cₛ-cₛ-cₛ-aₛ-t
where the lower case letter represents DNA and “s” represents phosphorothioate backbone;

(iv) antisense HIF1α LNA oligomer (SEQ ID NO: 5)
TₛGₛGₛcₛaₛaₛgₛcₛaₛtₛcₛcₛTₛGₛTₛa
where the upper case letter represents LNA and the “s” represents phosphorothioate backbone.

(v) antisense ErbB3 LNA oligomer (SEQ ID NO: 6)
TₛAₛGₛcₛcₛtₛgₛtₛcₛaₛcₛtₛtₛ^{Me}CₛTₛ^{Me}Cₛ
where the upper case letter represents LNA and the “s” represents phosphorothioate backbone.

(vi) antisense ErbB3 LNA oligomer (SEQ ID NO: 7)
Gₛ^{Me}CₛTₛcₛcₛaₛgₛaₛcₛaₛtₛcₛaₛ^{Me}CₛTₛ^{Me}C
where the upper case letter represents LNA and the “s” represents phosphorothioate backbone.

(vii) antisense PIK3CA LNA oligomer (SEQ ID NO: 8)
AₛGₛ^{Me}CₛcₛaₛtₛtₛcₛaₛtₛtₛcₛcₛAₛ^{Me}Cₛ^{Me}C
where the upper case letter represents LNA and the “s” represents phosphorothioate backbone.

(viii) antisense PIK3CA LNA oligomer (SEQ ID NO: 9)
TₛTₛAₛtₛtₛgₛtₛgₛcₛaₛtₛcₛtₛ^{Me}CₛAₛG
where the upper case letter represents LNA and the “s” represents phosphorothioate backbone.

(ix) antisense HSP27 LNA oligomer (SEQ ID NO: 10)
C_{S}G_{S}T_{S}g_{S}t_{S}a_{S}t_{S}t_{S}t_{S}c_{S}c_{S}g_{S}e_{S}G_{S}T_{S}G
where the upper case letter represents LNA and the “s” represents phosphorothioate backbone.

(x) antisense HSP27 LNA oligomer (SEQ ID NO: 11)
GₛGₛ^{Me}CₛaₛcₛaₛgₛcₛcₛaₛgₛtₛgₛGₛ^{Me}CₛG
where the upper case letter represents LNA and the “s” represents phosphorothioate backbone.

(xi) antisense Androgen Receptor LNA oligomer (SEQ ID NO: 12)
^{Me}Cₛ^{Me}Cₛ^{Me}CₛaₛaₛgₛgₛcₛaₛcₛtₛgₛcₛAₛGₛA
where the upper case letter represents LNA and the “s” represents phosphorothioate backbone.

(xii) antisense Androgen Receptor LNA oligomer (SEQ ID NO: 13)
Aₛ^{Me}Cₛ^{Me}CₛaₛaₛgₛtₛtₛtₛcₛtₛtₛcₛAₛGₛ^{Me}C
where the upper case letter represents LNA and the “s” represents phosphorothioate backbone.

(xiii) antisense GLI2 LNA oligomer (SEQ ID NO: 14)
^{Me}C_{S}T_{S}^{Me}C_{S}c_{S}t_{S}t_{S}g_{S}g_{S}t_{S}g_{S}c_{S}a_{S}g_{S}T_{S}^{Me}C_{S}T
where the upper case letter represents LNA and the “s” represents phosphorothioate backbone.

(xiv) antisense GLI2 LNA oligomer (SEQ ID NO: 15)
Tₛ^{Me}CₛAₛgₛaₛtₛtₛcₛaₛaₛaₛcₛ^{Me}Cₛ^{Me}CₛA
where the upper case letter represents LNA and the “s” represents phosphorothioate backbone

(xv) antisense beta-catenin LNA oligomer (SEQ ID NO: 16)
GₛTₛGₛtₛtₛcₛtₛaₛcₛaₛcₛcₛaₛTₛTₛA
where the upper case letter represents LNA and the “s” represents phosphorothioate backbone.

Lower case letters represent DNA units, bold upper case letters represent LNA such as β-D-oxy-LNA units. All cytosine bases in the LNA monomers are 5-methylcytosine. Subscript “s” represents phosphorothioate linkage.

LNA includes 2′-O, 4′-C methylene bicyclonudeotide as shown below:

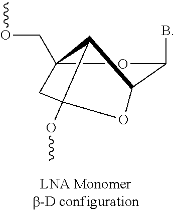

See detailed description of Survivin LNA disclosed in U.S. patent application Ser. No. 11/272,124, entitled “LNA Oligonucleotides and the Treatment of Cancer” and Ser. No. 10/776,934, entitled “Oligomeric Compounds for the Modulation Survivin Expression”, the contents of each of which is incorporated herein by reference. See also U.S. Pat. No. 7,589,190 and U.S. Patent Publication No. 2004/0096848 for HIF-1α modulation; U.S. Patent Publication No. 2008/0318894 and PCT/US09/063357 for ErbB3 modulation; U.S. Patent Publication No. 2009/0192110 for PIK3CA modulation; PCT/IB09/052860 for HSP27 modulation; U.S. Patent Publication No. 2009/0181916 for Androgen Receptor modulation; and U.S. Provisional Application No. 61/081,135 and PCT Application No. PCT/IB09/006407, entitled “RNA Antagonists Targeting GLI2”; and U.S. Patent Publication Nos. 2009/0005335 and 2009/0203137 for Beta Catenin modulation; the contents of each which are also incorporated herein by reference. Additional examples of suitable target genes are described in WO 03/74654, PCT/US03/05028, and U.S. patent application Ser. No. 10/923,536, the contents of which are incorporated by reference herein.

In a further embodiment, the nanoparticle described herein can include oligonucleotides releasably linked to an endosomal release-promoting group. The endosomal release-promoting groups such as histidine-rich peptides can disrupt the endosomal membrane, thereby facilitating cytoplasmic delivery of therapeutic agents. Histidine-rich peptides enhance endosomal release of oligonucleotides to the cytoplasm. Then, the intracellularly released oligonucleotides can translocate to the nucleus. Additional details of oligonucleotide-histidine rich peptide conjugates are described in U.S. Provisional Patent Application Ser. Nos. 61/115,350 and 61/115,326 filed Nov. 17, 2008, and PCT Patent Application No. ______, filed on even date, and entitled “Releasable Conjugates For Nucleic Acids Delivery Systems”, the contents of each of which are incorporated herein by reference.

### 6. Targeting Groups

Optionally/preferably, the nanoparticle compositions described herein further include a targeting ligand for a specific cell or tissue type. The targeting group can be attached to any component of a nanoparticle composition (preferably, fusogenic lipids and PEG-lipids) using a linker molecule, such as an amide, amido, carbonyl, ester, peptide, disulphide, silane, nucleoside, abasic nucleoside, polyether, polyamine, polyamide, peptide, carbohydrate, lipid, polyhydrocarbon, phosphate ester, phosphoramidate, thiophosphate, alkylphosphate, maleimidyl linker or photolabile linker. Any known techniques in the art can be used for conjugating a targeting group to any component of the nanoparticle composition without undue experimentation.

For example, targeting agents can be attached to the polymeric portion of PEG lipids to guide the nanoparticles to the target area in vivo. The targeted delivery of the nanoparticle described herein enhances the cellular uptake of the nanoparticles encapsulating therapeutic nucleic acids, thereby improving the therapeutic efficacies. In certain aspects, some cell penetrating peptides can be replaced with a variety of targeting peptides for targeted delivery to the tumor site.

In one preferred aspect of the invention, the targeting moiety, such as a single chain antibody (SCA) or single-chain antigen-binding antibody, monoclonal antibody, cell adhesion peptides such as RGD peptides and Selectin, cell penetrating peptides (CPPs) such as TAT, Penetratin and (Arg)₉, receptor ligands, targeting carbohydrate molecules or lectins allows nanoparticles to be specifically directed to targeted regions. See J Pharm Sci. 2006 September; 95(9):1856-72 Cell adhesion molecules for targeted drug delivery, the contents of which are incorporated herein by reference.

Preferred targeting moieties include single-chain antibodies (SCAB) or single-chain variable fragments of antibodies (sFv). The SCA contains domains of antibodies which can bind or recognize specific molecules of targeting tumor cells. In addition to maintaining an antigen binding site, a SCA conjugated to a PEG-lipid can reduce antigenicity and increase the half life of the SCA in the bloodstream.

The terms “single chain antibody” (SCA), “single-chain antigen-binding molecule or antibody” or “single-chain Fv” (sFv) are used interchangeably. The single chain antibody has binding affinity for the antigen. Single chain antibody (SCA) or single-chain Fvs can and have been constructed in several ways. A description of the theory and production of single-chain antigen-binding proteins is found in commonly assigned U.S. patent application Ser. No. 10/915,069 and U.S. Pat. No. 6,824,782, the contents of each of which are incorporated by reference herein.

Typically, SCA or Fv domains can be selected among monoclonal antibodies known by their abbreviations in the literature as 26-10, MOPC 315, 741F8, 520C9, McPC 603, D1.3, murine phOx, human phOx, RFL3.8 sTCR, 1A6, Se155-4,18-2-3,4-4-20,7A4-1, B6.2, CC49,3C2,2c, MA-15C5/K₁₂G_{O}, Ox, etc. (see, Huston, J. S. et al., Proc. Natl. Acad. Sci. USA 85:5879-5883 (1988); Huston, J. S. et al., SIM News 38(4) (Supp):11 (1988); McCartney, J. et al., ICSU Short Reports 10:114 (1990); McCartney, J. E. et al., unpublished results (1990); Nedelman, M. A. et al., J. Nuclear Med. 32 (Supp.):1005 (1991); Huston, J. S. et at, In: Molecular Design and Modeling: Concepts and Applications, Part B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem. 265:18615-18620 (1990); Colcher, D. et al., J. Nat. Cancer Inst. 82:1191-1197 (1990); Gibbs, R. A. et al., Proc. Natl. Acad. Sci. USA 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. USA 87:1066-1070 (1990); Batra, J. K. et al., Biochem. Biophys. Res. Comm. 171:1-6 (1990); Batra, J. K. et al., J. Biol. Chem. 265:15198-15202 (1990); Chaudhary, V. K. et al., Proc. Natl. Acad Sci. USA 87:9491-9494 (1990); Batra, J. K. et al., Mol. Cell. Biol. 11:2200-2205 (1991); Brinkmann, U. et al., Proc. Natl. Acad. Sci. USA 88:8616-8620 (1991); Seetharam, S. et al., J. Biol. Chem. 266:17376-17381 (1991); Brinkmann, U. et al., Proc. Natl. Acad. Sci. USA 89:3075-3079 (1992); Glockshuber, R. et at, Biochemistry 29:1362-1367 (1990); Skerra, A. et al., Bio/Technol. 9:273-278 (1991); Pack, P. et al., Biochemistry 31:1579-1534 (1992); Clackson, T. et at, Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. USA 87:6654-6658 (1990); Condra, J. H. et al., J. Biol, Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Biol Technol. 9:1369-1372 (1991); Breitling, F. et at, Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. USA 88:8646-8650 (1991); Traunecker, A. et al., EMBO J. 10:3655-3659 (1991); Hoc, W. F. S. et al., Proc. Natl. Acad. Sci. USA 89:4759-4763 (1993)). Each of the foregoing publications is incorporated herein by reference.

A non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides, RGD peptides, folate, anisamide, etc. Other optional targeting agents appreciated by artisans in the art can be also employed in the nanoparticles described herein.

In one preferred embodiment, the targeting agents useful for the compounds described herein include single chain antibody (SCA), RGD peptides, selectin, TAT, penetratin, (Arg)₉, folic acid, anisamide, etc., and some of the preferred structures of these agents are:

| | |
|---|---|
| C-TAT: (SEQ ID NO: 17) | CYGRKKRRQRRR; |
| C-(Arg)₉: (SEQ ID NO: 18) | CRRRRRRRRR; |

RGD can be linear or cyclic:

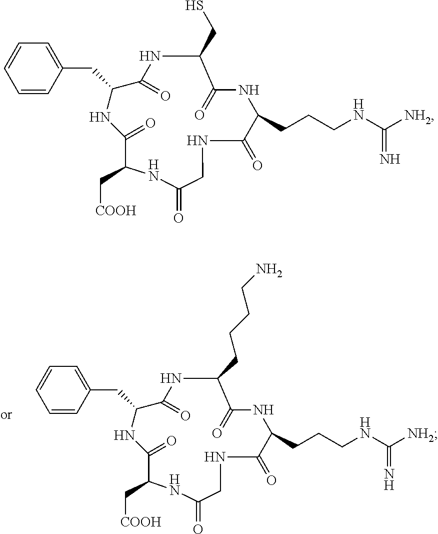

Folic acid is a residue of

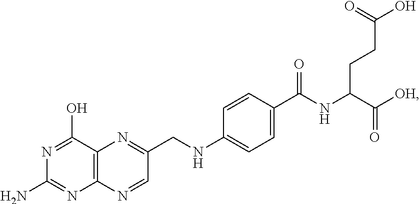

and

Anisamide is p-MeO-Ph-C(═O)OH.

Arg₉ can include a cysteine for conjugating such as CRRRRRRRRR and TAT can add an additional cysteine at the end of the peptide such as CYGRKKRRQRRRC.

For purpose of the current invention, the abbreviations used in the specification and figures represent the following structures.:

(i) C-diTAT (SEQ ID NO: 19)=CYGRKKRRQRRRYGRKKRRQRRR—NH₂;

(ii) Linear RGD (SEQ ID NO: 20)=RGDC;

(iii) Cyclic RGD (SEQ ID NO: 21 and SEQ ID NO: 22)=c-RGDFC or c-RGDFK;

(iv) RGD-TAT (SEQ ID NO: 23)=CYGRKKRRQRRRGGGRGDS—NH₂ ; and

(v) Arg₉ (SEQ ID NO: 24)=RRRRRRRRR.

Alternatively, the targeting group include sugars and carbohydrates such as galactose, galactosamine, and N-acetyl galactosamine; hormones such as estrogen, testosterone, progesterone, glucocortisone, adrenaline, insulin, glucagon, cortisol, vitamin D, thyroid hormone, retinoic acid, and growth hormones; growth factors such as VEGF, EGF, NGF, and PDGF; neurotransmitters such as GABA, Glutamate, acetylcholine; NOGO; inostitol triphosphate; epinephrine; norepinephrine; Nitric Oxide, peptides, vitamins such as folate and pyridoxine, drugs, antibodies and any other molecule that can interact with a cell surface receptor in vivo or in vitro.

### D. Preparation of Nanoparticles

The nanoparticle described herein can be prepared by any art-known process without undue experimentation.

For example, the nanoparticle can be prepared by providing nucleic acids such as oligonucleotides in an aqueous solution (or an aqueous solution without nucleic acids for comparison study) in a first reservoir, providing an organic lipid solution containing the nanoparticle composition described herein in a second reservoir, and mixing the aqueous solution with the organic lipid solution such that the organic lipid solution mixes with the aqueous solution to produce nanoparticles encapsulating the nucleic acids. Details of the process are described in U.S. Patent Publication No. 2004/0142025, the contents of which are incorporated herein by reference.

Alternatively, the nanoparticles described herein can be prepared by using any methods known in the art including, e.g., a detergent dialysis method or a modified reverse-phase method which utilizes organic solvents to provide a single phase during mixing the components. In a detergent dialysis method, nucleic acids (i.e., siRNA) are contacted with a detergent solution of cationic lipids to form a coated nucleic acid complex.

In one embodiment of the invention, the cationic lipids and nucleic acids such as oligonucleotides are combined to produce a charge ratio of from about 1:20 to about 20:1, preferably in a ratio of from about 1:5 to about 5:1, and more preferably in a ratio of from about 1:2 to about 2:1.

In one embodiment of the invention, the cationic lipids and nucleic acids such as oligonucleotides are combined to produce a charge ratio of from about 1:1 to about 20:1, from about 1:1 to about 12:1, and more preferably in a ratio of from about 2:1 to about 6:1. Alternatively, the nitrogen to phoshpate (N/P) ratio of the nanoparticle composition ranges from about 2:1 to about 5:1, (i.e., 2.5:1).

In another embodiment, the nanoparticle described herein can be prepared by using a dual pump system. Generally, the process includes providing an aqueous solution containing nucleic acids in a first reservoir and a lipid solution containing the nanoparticle composition described in a second reservoir. The two solutions are mixed by using a dual pump system to provide nanoparticles. The resulting mixed solution is subsequently diluted with an aqueous buffer and the nanoparticles formed can be purified and/or isolated by dialysis. The nanoparticles can be further processed to be sterilized by filtering through a 0.22 μm filter.

The nanoparticles containing nucleic acids range from about 5 to about 300 nm in diameter. Preferably, the nanoparticles have a median diameter of less than about 150 nm (e.g., about 50-150 nm), more preferably a diameter of less than about 100 nm, by the measurement using the Dynamic Light Scattering technique (DLS). A majority of the nanoparticles have a median diameter of about 30 to 100 nm (e.g., 59.5, 66, 68, 76, 80, 93, 96 nm), preferably about 60 to about 95 nm. Artisans will appreciate that the measurement using other art-known techniques such as TEM may provide a median diameter number decreased by half, as compared to the DLS technique. The nanoparticles of the present invention are substantially uniform in size as shown by polydispersity.

Optionally, the nanoparticles can be sized by any methods known in the art. The size can be controlled as desired by artisans. The sizing may be conducted in order to achieve a desired size range and relatively narrow distribution of nanoparticle sizes. Several techniques are available for sizing the nanoparticles to a desired size. See, for example, U.S. Pat. No. 4,737,323, the contents of which are incorporated herein by reference.

The present invention provides methods for preparing serum-stable nanoparticles such that nucleic acids (e.g., LNA or siRNA) are encapsulated in a lipid multi-lamellar structure (i.e. a lipid bilayer) and are protected from degradation. The nanoparticles described herein are stable in an aqueous solution. Nucleic acids included in the nanoparticles are protected from nucleases present in the body fluid.

Additionally, the nanoparticles prepared according to the present invention are preferably neutral or positively-charged at physiological pH.

The nanoparticle or nanoparticle complex prepared using the nanoparticle composition described herein includes: (i) a cationic lipid; (ii) a fusogenic lipid including a compound of Formula (I); (iii) a PEG-lipid and (iv) nucleic acids such as an oligonucleotide.

In one embodiment, the nanoparticle composition includes a mixture of a cationic lipid, a compound of Formula (I) optionally with a diacylphosphatidylethanolamine, a PEG conjugated to phosphatidylethanolamine (PEG-PE), and cholesterol;

a cationic lipid, a compound of Formula (I) optionally with a diacylphosphatidylcholine, a PEG conjugated to phosphatidylethanolamine (PEG-PE), and cholesterol;

a cationic lipid, a compound of Formula (I) optionally with a diacylphosphatidylethanolamine, a diacylphosphatidyl-choline, a PEG conjugated to phosphatidylethanolamine (PEG=PE), and cholesterol;

a cationic lipid, a compound of Formula (I) optionally with a diacylphosphatidylethanolamine, a PEG conjugated to ceramide (PEG-Cer), and cholesterol; and

a cationic lipid, a compound of Formula (I) optionally with a diacylphosphatidylethanolamine, a PEG conjugated to phosphatidylethanolamine (PEG-PE), a PEG conjugated to ceramide (PEG-Cer), and cholesterol.

Additional nanoparticle compositions can be prepared by modifying compositions containing art-known cationic lipid(s). Nanoparticle compositions containing a compound of Formula (I) can be modified by adding art-known cationic lipids. See art-known compositions described in Table IV of US Patent Application Publication No. 2008/0020058, the contents of which are incorporated herein by reference.

A non-limiting list of nanoparticle compositions for the preparation of nanoparticles is set forth in Table 3.

TABLE 3

| Sample | | | |
|---|---|---|---|
| No. | Nanoparticle Composition | Molar Ratio | Oligo |
| 1 | Cationic Lipid 1:cpd 10:DSPC:Chol:DSPE-PEG | 15:15:20:40:10 | Oligo-1 |
| 2 | Cationic Lipid 1:cpd 10:DSPC:Chol:DSPE-PEG | 15:5:20:50:10 | Oligo-1 |
| 3 | Cationic Lipid 1:cpd 10:DSPC:Chol:DSPE-PEG | 25:15:20:30:10 | Oligo-1 |
| 4 | Cationic Lipid 1:cpd 10:Chol:DSPE-PEG | 20:47:30:3 | Oligo-1 |
| 5 | Cationic Lipid 1:cpd 10:Chol:DSPE-PEG | 17:60:20:3 | Oligo-1 |
| 6 | Cationic Lipid 1:cpd 10:DSPE-PEG | 20:78:2 | Oligo-1 |
| 7 | Cationic Lipid 1:cpd 10:Chol:C16mPEG-Ceramide | 17:60:20:3 | Oligo-2 |
| 8 | Cationic Lipid 1:cpd 10:Chol:DSPE-PEG:Cl6mPEG- | 18:60:20:1:1 | Oligo-2 |
| | Ceramide | | |

In one embodiment, the molar ratio of cationic lipid 1:compound 10:cholesterol:PEG-DSPE:C16mPEG-Ceramide in the nanoparticle is in a molar ratio of about 18%:60%:20%:1%:1%, respectively. (Sample. No. 8)

In another embodiment, the nanoparticle contains cationic lipid 1, compound 10, cholesterol and C16mPEG-Ceramide in a molar ratio of about 17%:60%:20%:3% of the total lipid present in the nanoparticle composition. (Sample No. 7)

In one embodiment, the cationic lipid contained in the compositions has the structure:

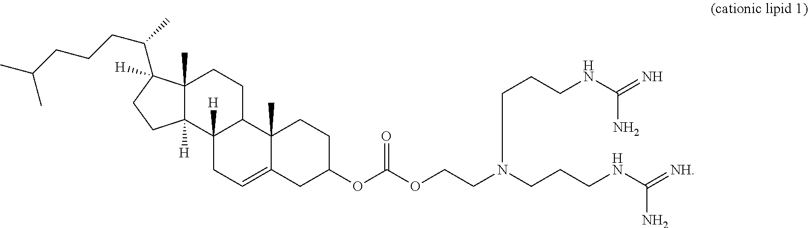

In a further embodiment, these nanoparticle compositions contain a releasable polymeric lipid having the structure:

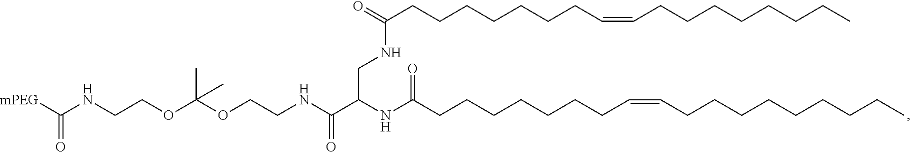

Wherein the polymer portion of the PEG lipid has a number average weight of about 2,000 daltons.

The molar ratio as used herein refers to the amount relative to the total lipid present in the nanoparticle composition.

### F. Methods of Treatment

The nanoparticles described herein can be employed in the treatment for preventing, inhibiting, reducing or treating any trait, disease or condition that is related to or responds to the levels of target gene expression in a cell or tissue, alone or in combination with other therapies. The methods include administering the nanoparticles described herein to a mammal in need thereof.

One aspect of the present invention provides methods of introducing or delivering therapeutic agents such as nucleic acids/oligonucleotides into a mammalian cell in vivo and/or in vitro.

The method according to the present invention includes contacting a cell with the compounds described herein. The delivery can be made in vivo as part of a suitable pharmaceutical composition or directly to the cells in an ex vivo or in vitro environment.

The present invention is useful for introducing oligonucleotides to a mammal. The compounds described herein can be administered to a mammal, preferably human.

According to the present invention, the present invention preferably provides methods of inhibiting, or downregulating (or modulating) gene expression in mammalian cells or tissues. The downregulation or inhibition of gene expression can be achieved in vivo, ex vivo and/or in vitro. The methods include contacting human cells or tissues with nanoparticles encapsulating nucleic acids or administering the nanoparticles to a mammal in need thereof. Once the contacting has occurred, successful inhibition or down-regulation of gene expression such as in mRNA or protein levels shall be deemed to occur when at least about 10%, preferably at least about 20% or higher (e.g., at least about 25%, 30%, 40%, 50%, 60%) is realized in vivo, ex vivo or in vitro when compared to that observed in the absence of the nanoparticles described herein.

For purposes of the present invention, “inhibiting” or “downregulating” shall be understood to mean that the expression of a target gene, or level of RNAs or equivalent RNAs encoding one or more protein subunits, or activity of one or more protein subunits is reduced when compared to that observed in the absence of the nanoparticles described herein.

In one preferred embodiment, a target gene includes, for example, but is not limited to, oncogenes, pro-angiogenesis pathway genes, pro-cell proliferation pathway genes, viral infectious agent genes, and pro-inflammatory pathway genes.

Preferably, gene expression of a target gene is inhibited in cancer cells or tissues, for example, brain, breast, colorectal, gastric, lung, mouth, pancreatic, prostate, skin or cervical cancer cells. The cancer cells or tissues can be from one or more of the following: solid tumors, lymphomas, small cell lung cancer, acute lymphocytic leukemia (ALL), pancreatic cancer, glioblastoma, ovarian cancer, gastric cancer, breast cancer, colorectal cancer, prostate cancer, cervical cancer, brain tumors, KB cancer, lung cancer, colon cancer, epidermal cancer, etc.

In one particular embodiment, the nanoparticles according to the methods described herein include, for example, antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and antisense beta-catenin oligonucleotides.

According to the present invention, the nanoparticles can include oligonucleotides (SEQ ID NO: 1, SEQ ID NOs 2 and 3, SEQ ID NO:3, SEQ ID NO: 4, SEQ ID NO: 5, SEQ ID NO: 6, SEQ ID NO: 7, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16 in which each nucleic acid is a naturally occurring or modified nucleic acid) can be used. The therapy contemplated herein uses nucleic acids encapsulated in the aforementioned nanoparticle. In one embodiment, therapeutic nucleotides containing eight or more consecutive antisense nucleotides can be employed in the treatment.

Alternatively, there are also provided methods of treating a mammal. The methods include administering an effective amount of a pharmaceutical composition containing a nanoparticle described herein to a patient in need thereof. The efficacy of the methods would depend upon efficacy of the nucleic acids for the condition being treated. The present invention provides methods of treatment for various medical conditions in mammals. The methods include administering, to the mammal in need of such treatment, an effective amount of a nanoparticle containing encapsulated therapeutic nucleic acids. The nanoparticles described herein are useful for, among other things, treating diseases such as (but not limited to) cancer, inflammatory disease, and autoimmune disease.

In one embodiment, there are also provided methods of treating a patient having a malignancy or cancer, comprising administering an effective amount of a pharmaceutical composition containing the nanoparticle described herein to a patient in need thereof. The cancer being treated can be one or more of the following: solid tumors, lymphomas, small cell lung cancer, acute lymphocytic leukemia (ALL), pancreatic cancer, glioblastoma, ovarian cancer, gastric cancers, colorectal cancer, prostate cancer, cervical cancer, brain tumors, KB cancer, lung cancer, colon cancer, epidermal cancer, etc. The nanoparticles are useful for treating neoplastic disease, reducing tumor burden, preventing metastasis of neoplasms and preventing recurrences of tumor/neoplastic growths in mammals by downregulating gene expression of a target gene. For example, the nanoparticles are useful in the treatment of metastatic disease (i.e. cancer with metastasis into the liver).

In yet another aspect, the present invention provides methods of inhibiting the growth or proliferation of cancer cells in vivo or in vitro. The methods include contacting cancer cells with the nanoparticle described herein. In one embodiment, the present invention provides methods of inhibiting the growth of cancer in vivo or in vitro wherein the cells express ErbB3 gene.

In another aspect, the present invention provides a means to deliver nucleic acids (e.g., antisense ErbB3 LNA oligonucleotides) inside a cancer cell where it can bind to ErbB3 mRNA, e.g., in the nucleus. As a consequence, the ErbB3 protein expression is inhibited, which inhibits the growth of the cancer cells. The methods introduce oligonucleotides (e.g. antisense oligonucleotides including LNA) to cancer cells and reduce target gene (e.g., survivin, HIF-1α or ErbB3) expression in the cancer cells or tissues.

Alternatively, the present invention provides methods of modulating apoptosis in cancer cells. In yet another aspect, there are also provided methods of increasing the sensitivity of cancer cells or tissues to chemotherapeutic agents in vivo or in vitro.

In yet another aspect, there are provided methods of killing tumor cells in vivo or in vitro. The methods include introducing the compounds described herein to tumor cells to reduce gene expression such as ErbB3 gene and contacting the tumor cells with an amount of at least one anticancer agent (e.g., a chemotherapeutic agent) sufficient to kill a portion of the tumor cells. Thus, the portion of tumor cells killed can be greater than the portion which would have been killed by the same amount of the chemotherapeutic agent in the absence of the nanoparticles described herein.

In a further aspect of the invention, an anticancer/chemotherapeutic agent can be used in combination, simultaneously or sequentially, with the compounds described herein. The compounds described herein can be administered prior to, or concurrently with, the anticancer agent, or after the administration of the anticancer agent. Thus, the nanoparticles described herein can be administered prior to, during, or after treatment of the chemotherapeutic agent.

Still further aspects include combining the compound of the present invention described herein with other anticancer therapies for synergistic or additive benefit.

Alternatively, the nanoparticle composition described herein can be used to deliver a pharmaceutically active agent, preferably having a negative charge or a neutral charge to a mammal. The nanoparticle encapsulating pharmaceutically active agents/compounds can be administered to a mammal in need thereof. The pharmaceutically active agents/compounds include small molecular weight molecules. Typically, the pharmaceutically active agents have a molecular weight of less than about 1,500 daltons (i.e., less than 1,000 daltons).

In a further embodiment, the compounds described herein can be used to deliver nucleic acids, a pharmaceutically active agent, or in combination thereof.

In yet a further embodiment, the nanoparticle associated with the treatment can contain a mixture of one or more therapeutic nucleic acids (either the same or different, for example, the same or different oligonucleotides), and/or one or more pharmaceutically active agents for synergistic application.

### G. Pharmaceutical Compositions/Formulations of Nanoparticles

Pharmaceutical compositions/formulations including the nanoparticles described herein may be formulated in conjunction with one or more physiologically acceptable carriers comprising excipients and auxiliaries which facilitate processing of the active compounds into preparations which can be used pharmaceutically. Proper formulation is dependent upon the route of administration chosen, i.e., whether local or systemic treatment is treated.

Suitable forms, in part, depend upon the use or the route of entry, for example oral, transdermal, or injection. Factors for considerations known in the art for preparing proper formulations include, but are not limited to, toxicity and any disadvantages that would prevent the composition or formulation from exerting its effect.

Administration of pharmaceutical compositions of nanoparticles described herein may be oral, pulmonary, topical or parentarel. Topical administration includes, without limitation, administration via the epidermal, transdermal, ophthalmic routes; including via mucous membranes, e.g., including vaginal and rectal delivery. Parenteral administration, including intravenous, intraarterial, subcutaneous, intraperitoneal or intramuscular injection or infusion, is also contemplated.

In one preferred embodiment, the nanoparticles containing therapeutic oligonucleotides are administered intravenously (i.v.) or intraperitoneally (i.p.). Parenteral routes are preferred in many aspects of the invention.

For injection, including, without limitation, intravenous, intramuscular and subcutaneous injection, the nanoparticles of the invention may be formulated in aqueous solutions, preferably in physiologically compatible buffers such as physiological saline buffer or polar solvents including, without limitation, a pyrrolidone or dimethylsulfoxide.

The nanoparticles may also be formulated for bolus injection or for continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form. Aqueous injection suspensions may contain substances that modulate the viscosity of the suspension, such as sodium carboxymethyl cellulose, sorbitol, or dextran. Optionally, the suspension may also contain suitable stabilizers and/or agents that increase the concentration of the nanoparticles in the solution. Alternatively, the nanoparticles may be in powder form for constitution with a suitable vehicle, e.g., sterile, pyrogen-free water, before use.

For oral administration, the nanoparticles described herein can be formulated by combining the nanoparticles with pharmaceutically acceptable carriers well-known in the art. Such carriers enable the nanoparticles of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars (for example, lactose, sucrose, mannitol, or sorbitol), cellulose preparations such as maize starch, wheat starch, rice starch and potato starch and other materials such as gelatin, gum tragacanth, methyl cellulose, hydroxypropylmethylcellulose, sodium carboxymethylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the nanoparticles of the present invention can conveniently be delivered in the form of an aerosol spray using a pressurized pack or a nebulizer and a suitable propellant.

The nanoparticles may also be formulated in rectal compositions such as suppositories or retention enemas, using, e.g., conventional suppository bases such as cocoa butter or other glycerides.

In addition to the formulations described previously, the nanoparticles may also be formulated as depot preparations. Such long acting formulations may be administered by implantation (for example, subcutaneously or intramuscularly) or by intramuscular injection. A nanoparticle of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Additionally, the nanoparticles may be delivered using a sustained-release system, such as semi-permeable matrices of solid hydrophobic polymers containing the nanoparticles. Various sustained-release materials have been established and are well known by those skilled in the art.

In addition, antioxidants and suspending agents can be used in the pharmaceutical compositions of the nanoparticles described herein.

### H. Dosages

Determination of doses adequate to inhibit the expression of one or more preselected genes, such as a therapeutically effective amount in the clinical context, is well within the capability of those skilled in the art, especially in light of the disclosure herein.

For any therapeutic nucleic acids used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the pharmaceutical composition that is administered will depend upon the potency of the nucleic acids included therein. Generally, the amount of the nanoparticles containing nucleic acids used in the treatment is that amount which effectively achieves the desired therapeutic result in mammals. Naturally, the dosages of the various nanoparticles will vary somewhat depending upon the nucleic acids (or pharmaceutically active agents) encapsulated therein (e.g., oligonucleotides). In addition, the dosage, of course, can vary depending upon the dosage form and route of administration. In general, however, the nucleic acids encapsulated in the nanoparticles described herein can be administered in amounts ranging from about 0.1 to about 1 g/kg/week, preferably from about 1 to about 500 mg/kg and more preferably from 1 to about 100 mg/kg (i.e., from about 3 to about 90 mg/kg/dose).

The range set forth above is illustrative and those skilled in the art will determine the optimal dosing based on clinical experience and the treatment indication. Moreover, the exact formulation, route of administration and dosage can be selected by the individual physician in view of the patient's condition. Additionally, toxicity and therapeutic efficacy of the nanoparticles described herein can be determined by standard pharmaceutical procedures in cell cultures or experimental animals using methods well-known in the art.

Alternatively, an amount of from about 1 mg to about 100 mg/kg/dose (0.1 to 100 mg/kg/dose) can be used in the treatment depending on potency of the nucleic acids. Dosage unit forms generally range from about 1 mg to about 60 mg of an active agent, oligonucleotides.

In one embodiment, the treatment of the present invention includes administering the nanoparticles described herein in an amount of from about 1 to about 60 mg/kg/dose (from about 25 to 60 mg/kg/dose, from, about 3 to about 20 mg/kg/dose), such as 60, 45, 35, 30, 25, 15, 5 or 3 mg/kg/dose (either in a single or multiple dose regime) to a mammal. For example, the nanoparticles described herein can be administered introvenously in an amount of 5, 25, 30, or 60 mg/kg/dose at q3d×9. For another example, the treatment protocol includes administering an antisense oligonucleotide in an amount of from about 4 to about 18 mg/kg/dose weekly, or about 4 to about 9.5 mg/kg/dose weekly (e.g., about 8 mg/kg/dose weekly for 3 weeks in a six week cycle).

Alternatively, the delivery of the oligonucleotide encapsulated within the nanoparticles described herein includes contacting a concentration of oligonucleotides of from about 0.1 to about 1000 μM, preferably from about 10 to about 1500 μM (i.e. from about 10 to about 1000 μM, from about 30 to about 1000 μM) with tumor cells or tissues in vivo, ex vivo or in vitro.

The compositions may be administered once daily or divided into multiple doses which can be given as part of a multi-week treatment protocol. The precise dose will depend on the stage and severity of the condition, the susceptibility of the disease such as tumor to the nucleic acids, and the individual characteristics of the patient being treated, as will be appreciated by one of ordinary skill in the art.

In all aspects of the invention where nanoparticles are administered, the dosage amount mentioned is based on the amount of oligonucleotide molecules rather than the amount of nanoparticles administered.

It is contemplated that the treatment will be given for one or more days until the desired clinical result is obtained. The exact amount, frequency and period of administration of the nanoparticles encapsulating therapeutic nucleic acids (or pharmaceutically active agents) will vary, of course, depending upon the sex, age and medical condition of the patent as well as the severity of the disease as determined by the attending clinician.

Still further aspects include combining the nanoparticles of the present invention described herein with other anticancer therapies for synergistic or additive benefit.

### EXAMPLES

The following examples serve to provide further appreciation of the invention but are not meant in any way to restrict the effective scope of the invention.

In the examples, all synthesis reactions are run under an atmosphere of dry nitrogen or argon. N-(3-aminopropyl)-1,3-propanediamine), BOC—ON, LiOCl₄, Cholesterol and 1H-Pyrazole-1-carboxamidine.HCl were purchased from Aldrich. All other reagents and solvents were used without further purification. An LNA Oligo-1 targeting survivin gene, and Oligo-2 targeting ErbB3 gene were prepared in house and their sequences are given in Table 4. The internucleosides linkage is phosphorothioate, ^{m}C represents methylated cytosine, and the upper case letters indicate LNA.

TABLE 4

| LNA Oligo | Sequence |
|---|---|
| Oligo-1 (SEQ ID NO: 1) | 5′-^{m}CT^{m}CAatccatgg^{m}CAGc-3′ |
| Oligo-2 (SEQ ID NO: 6) | 5′-TAGcctgtcactt^{m}CT^{m}C-3′ |

The following abbreviations may be used throughout the examples such as, LNA (Locked nucleic acid oligonucleotide), BACC (2-[N,N′-di(2-guanidiniumpropyl)]aminoethyl-cholesteryl-carbonate), Chol (cholesterol), DIEA (diisopropylethylamine), DMAP (4-N,N-dimethylamino-pyridine), DOPE (L-α-dioleoyl phosphatidylethanolamine, Avanti Polar Lipids, USA or NOF, Japan), DLS (Dynamic Light Scattering), DSPC (1,2-distearoyl-sn-glycero-3-phosphocholine) (NOF, Japan), DSPE-PEG (1,2-distearoyl-sn-glycero-3-phosphoethanolamine-N-(polyethylene glycol)2000 ammonium salt or sodium salt, Avanti Polar Lipids, USA and NOF, Japan), KD (knowndown), EPC (egg phosphatidylcholine, Avanti Polar Lipids, USA) and C16mPEG-Ceramide (N-palmitoyl-sphingosine-1-succinyl(methoxypolyethylene glycol)2000, Avanti Polar Lipids, USA). Other abbreviations such as the FAM (6-carboxyfluorescein), FBS (fetal bovine serum), GAPDH (glyceraldehyde-3-phosphate dehydrogenase), DMEM (Dulbecco's Modified Eagle's Medium), MEM (Modified Eagle's Medium), TEAA (tetraethylammonium acetate), TFA (trifluoroacetic acid), RT-qPCR (reverse transcription-quantitative polymerase chain reaction) may be also used.

### Example 1

### General NMR Method

¹H NMR spectra were obtained at 300 MHz and ¹³C NMR spectra at 75.46 MHz using a Varian Mercury300 NMR spectrometer and deuterated chloroform as the solvents unless otherwise specified. Chemical shifts (δ) are reported in parts per million (ppm) downfield from tetramethylsilane (TMS).

### Example 2

### General HPLC Method

The reaction mixtures and the purity of intermediates and final products are monitored by a Beckman Coulter System Gold® HPLC instrument. It employs a ZORBAX® 300SB C8 reversed phase column (150×4.6 mm) or a Phenomenex Jupiter® 300A C18 reversed phase column (150×4.6 mm) with a 168 Diode Array UV Detector, using a gradient of 10-90% of acetonitrile in 0.05% TFA at a flow rate of 1 mL/minute or a gradient of 25-35% acetonitrile in 50 mM TEAA buffer at a flow rate of 1 mL/minute. The anion exchange chromatography was run on AKTA explorer 100A from GE healthcare (Amersham Biosciences) using Poros 50HQ strong anion exchange resin from Applied Biosystems packed in an AP-Empty glass column from Waters. Desalting was achieved by using HiPrep 26/10 desalting columns from Amersham Biosciences. (for PEG-Oligo)

### Example 3

### General mRNA Down-Regulation Procedure

The cells are maintained in complete medium (F-12K or DMEM, supplemented with 10% FBS). A 12 well plate containing 2.5×10⁵ cells in each well is incubated overnight at 37° C. Cells are washed once with Opti-MEM® and 400 μL of Opti-MEM® is added per each well. Then, a solution of nanoparticle or Lipofectamine2000® containing oligonucleotide is added to each well. The cells is incubated for 4 hours, followed by addition of 600 μL of media per well, and incubation for 24 hours. After 24 hours of treatment, the intracellular mRNA levels of the target gene, such as human survivin, and a housekeeping gene, such as GAPDH are quantitated by RT-qPCR. The expression levels of mRNA are normalized.

### Example 4

### General RNA Preparation Procedure

For the in vitro mRNA down-regulation screen, total RNA is prepared using RNAqueous Kit® (Ambion) following the manufacturer's instruction. The RNA concentrations are determined by OD_{260 nm} using Nanodrop.

### Example 5

### General RT-qPCR Procedure

All the reagents are from Applied Biosystems: High Capacity cDNA Reverse Transcription Kit® (4368813), 20× PCR master mix (4304437), and TaqMan® Gene Expression Assays kits for human GAPDH (Cat. #0612177) and survivin (BIRKS Hs00153353). 2.0 μg of total RNA is used for cDNA synthesis in a final volume of 50 μL. The reaction is conducted in a PCR thermocycler at 25° C. for 10 minutes, 37° C. for 120 minutes, 85° C. for 5 seconds and then stored at 4° C. Real-time PCR is conducted with the program of 50° C-2 minutes, 95° C.-10 minutes, and 95° C.-15 seconds/60° C.-1 minute for 40 cycles. For each qPCR reaction, 1 μL of cDNA is used in a final volume of 30 μL.

### Example 6

### Preparation of H-Dap-OMe:2HCl (Compound 1)

H-Dap-(Boc)-OMe:HCl (5 g, 19.63 mmol) was treated with 2M HCl in 1,4-dioxane (130 mL) for 30 minutes at room temperature. The solvents were removed in vacuo at 30-35° C. The residue was re-suspended in diethyl ether and filtered. Isolated solids were dried in vacuo over P₂O₅ to yield 3.4 g (90%) of product: ¹¹³C NMR (DMSO-d₆) δ 38.95, 49.99, 53.53, 66.37, 166.77.

### Example 7

### Preparation of Dioleoyl-Dap-OMe (Compound 2)

A solution of compound 1 (3.4 g, 17.8 mmol) in 26 mL of anhydrous DMF was added to a solution of oleic acid (22.5 mL, 20.0 g, 71.1 mmol) in 170 mL of anhydrous DCM. The mixture was cooled to 0 to 5° C., followed by addition of EDC (20.5 g, 106.7 mmol) and DMAP (28.2 g, 231.1 mmol). The reaction mixture was stirred overnight and allowed to warm to room temperature under nitrogen. Completion of reaction was monitored by TLC (DCM:MEOH=90:1, v/v). The reaction mixture was diluted with 200 mL of reagent grade of DCM and washed with 1N HCl (3×80 mL) and 0.5% aqueous NaHCO₃ (3×80 mL). The resulting organic layer was separated, dried over anhydrous magnesium sulfate and concentrated in vacuo at 30° C. The residue was purified by silica gel column chromatography (DCM/MeOH/TEA 95:5:0.1, v/v/v) to yield 7.0 g (61%) of product: ¹³C NMR δ 14.15, 22.60, 25.55, 25.69, 27.20, 27.25, 29.18, 29.23, 29.29, 29.34, 29.55, 29.75, 29.78, 31.91, 36.43, 36.52, 41.53, 52.63, 53.58, 129.49, 129.54, 129.82, 129.85, 170.55, 173.59, 174.49.

### Example 8

### Preparation of Dialeoyl-Dap-OH (Compound 3)

A solution of NaOH (0.87 g, 21.63 mmol) in 7 mL of water was added to a solution of compound 2 (7.0 g, 10.8 mmol) in 70 mL of ethanol. The mixture was stirred at room temperature overnight and concentrated in vacuo at room temperature. The residue was suspended in 63 mL of water and the solution was acidified with 1N HCl at 0 to 5° C. The aqueous solution was extracted with DCM three times. Resulting organic layers were combined and dried over anhydrous magnesium sulfate. The solvent was removed in vacuo at 35° C. to yield 5.5 g (80%) of product: ¹³C NMR δ 14.19, 22.75, 25.51, 25.68, 27.25, 27.29, 29.21, 29.26, 29.32, 29.38, 29.59, 29.79, 29.82, 31.95, 36.30, 36.37, 41.58, 55.15, 129.53, 129.91, 171.49, 175.67, 176.19.

### Example 9

### Preparation of BocNHCH₂CH₂NH₂ (Compound 4)

A solution of Boc-anhydride (60 g, 274.9 mmol) in 150 mL of anhydrous DCM was slowly added to a solution of ethane-1,2-diamine (41.3 g, 687.3 mmol,) in 250 mL of anhydrous THF and 200 mL of anhydrous DCM at 0-5° C. over 1.5 hours. The reaction mixture was stirred overnight while allowed to warm to room temperature. 300 mL of water was added to the mixture, which was concentrated under vacuum at 30° C. The resulting aqueous solution was washed with DCM (3×300 mL) and the organic layers were combined and extracted with 0.5 N HCl (3×300 mL). Aqueous layers were combined and pH was adjusted to 9-10 with 4N NaOH solution, followed by extraction with DCM (3×500 mL). Organic layers were combined and dried over anhydrous magnesium sulfate. The solvent was removed in vacuo at 35° C. to yield 17.6 g (40%) of product: ¹³C NMR δ 28.23, 41.67, 43.19, 78.77, 155.93.

### Example 10

### Preparation of Dioleoyl-Dap-NHCH₂CH₂NHBoc (Compound 5)

DMAP (6.2 g, 51.2 mmol) was added to a solution of compound 3 (5.4 g, 8.53 mmol) in 50 mL of anhydrous DMF and 400 mL of anhydrous DCM and the solution was cooled in an ice bath. Compound 4 (2.73 g, 17.1 mmol) and EDC (6.6 g, 34.1 mmol) were added to the solution and the solution was stirred overnight while warmed to room temperature. Completion of reaction was monitored by TLC (DCM/MeOH=9:1, v/v) and the reaction mixture was diluted with 500 mL of DCM, washed with 0.2 N HCl (3×500 mL) and water (3×500 mL), and dried over anhydrous magnesium sulfate. The solvent was removed in vacuo at 35° C. to yield 5.6 g (85%) of product: ¹³C NMR δ 14.16, 22.72, 25.52, 25.77, 27.23, 27.26, 28.43, 29.24, 29.35, 29.56, 29.79, 31.92, 36.50, 40.25, 40.38, 41.99, 55.22, 76.57-77.42 (CDCl₃), 79.41, 129.54, 129.86, 156.35, 170.44, 174.25, 175.35.

### Example 11

### Preparation of Dioleoyl-Dap-NHCH₂CH₂NH₂ (Compound 6)

Compound 5 (5.6 g, 7.2 mmol) was dissolved in 95 ml DCM and the solution was treated with 24 mL of trifluoroacetic acid for 30 minutes at room temperature. The solvent was removed in vacuo at room temperature and the residue was redissolved in 200 mL DCM. The solution was washed with water and with 1% NaHCO₃ several times until pH was 8-9. Organic layer was dried over anhydrous magnesium sulfate and the solvent was removed in vacuo at 30° C. to yield 4.13 g (85%) of product: ¹³C NMR δ 14.15, 22.70, 25.62, 25.77, 27.25, 29.24, 29.35, 29.55, 29.78, 31.91, 36.43, 41.53, 54.95, 129.48, 129.85, 170.99, 174.43, 175.33.

### Example 12

### Preparation of 4-(dimethyl acetal)benzoic acid (Compound 7)

4-Formyl benzoic acid (1.5 g, 10 mmol) was dissolved in 30 mL of anhydrous methanol followed by the addition of 1.0 M lithium tetrafluroroborate in acetonitrile (300 μL, 0.3 mmol), trimethyl orthoformate (1.38 g, 10 mmol). The reaction mixture was refluxed overnight. The solvent was removed and the residue was suspended in boiling hexane for 30 minutes. The mixture was cooled to room temperature and the solid was isolated by filtration to yield 1.5 g (77%) of product: ¹³C NMR (CD₃OD) δ 53.26, 103.88, 127.75, 130.47, 131.14, 144.29, 169.30.

### Example 13

### Preparation of Compound 8

FmocNH-Lys(OMe)-NH₂ (0.60 mmol) and DMAP (219.6 mg, 1.80 mmol) are dissolved in anhydrous DCM and anhydrous DMF. The mixture is cooled to 0-5° C., followed by the addition of EDC (345.6 mg, 1.80 mmol) and compound 7 (352.8 mg, 1.80 mmol). The reaction mixture is stirred at 0° C. to room temperature overnight under N₂. The solvent is removed and the residue is recrystallized from mixed solvent of DMF/IPA (10 mL/100 mL) to give the product.

### Example 14

### Preparation of Compound 9

The compound 8 (0.46 mmol) in 6.75 mL chloroform is treated with 1.68 mL of 86% formic acid at room temperature overnight. The solvent is removed and the residue is recrystallized from DCM/ethyl ether twice to give the product.

### Example 15

### Preparation of Compound 10

Compound 6 (0.30 mmol) is dissolved in 10 mL of anhydrous DCM and 2 mL of anhydrous DMF, followed by addition of compound 9 (1.0 g, 0.2 mmol), molecular sieves (2 g) and DIEA (25.8 mg, 0.2 mmol). The reaction mixture is stirred at room temperature overnight under N₂. The reaction mixture is filtered and the filtrate is concentrated in vacuo. The residue is recrystallized from acetonitrile-IPA. The very fine solid suspension is centrifuged to give the product: The compound is treated with piperidine to remove Fmoc to give amine. The amine intermediate is treated with NaOH to hydrolyze the methyl ester followed by acidification to prepare compound 10.

### Example 16

### Preparation of LNA-Lipid Nanoparticle Composition

In this example, nanoparticle compositions encapsulating various nucleic acids such as LNA-containing oligonucleotides are prepared. For example, cationic lipid 1, compound 10, Chol, DSPE-PEG and C₁₆mPEG-Ceramide are mixed at a molar ratio of 18:60:20:1:1 in 10 mL of 90% ethanol (total lipid 30 pinole). LNA oligonucleotides (0.4 μmole) are dissolved in 10 mL of 20 mM Tris buffer (pH 7.4-7.6). After being heated to 37° C., the two solutions are mixed together through a duel syringe pump and the mixed solution is subsequently diluted with 20 mL of 20 mM Tris buffer (300 mM NaCl, pH 7.4-7.6). The mixture is incubated at 37° C. for 30 minutes and dialyzed in 10 mM PBS buffer (138 mM NaCl, 2.7 mM KCl, pH 7.4). Stable particles are obtained after the removal of ethanol from the mixture by dialysis. The nanoparticle solution is concentrated by centrifugation. The nanoparticle solution is transferred into a 15 mL centrifugal filter device (Amicon Ultra-15, Millipore, USA). Centrifuge speed is at 3,000 rpm and temperature is at 4° C. during centrifugation. The concentrated suspension is collected after a given time and is sterilized by filtration through a 0.22 μm syringe filter (Millex-GV, Millipore, USA).

The diameter and polydispersity of nanoparticle are measured at 25° in water (Sigma) as a medium on a Plus 90 Particle Size Analyzer Dynamic Light Scattering Instrument (Brookhaven, N.Y.).

Encapsulation efficiency of LNA oligonucleotides is determined by UV-VIS (Agilent 8453). The background UV-vis spectrum is obtained by scanning solution, which is a mixed solution composed of PBS buffer saline (250 μL), methanol (625 μL) and chloroform (250 μL). In order to determine the encapsulated nucleic acids concentration, methanol (625 μL) and chloroform (250 μL) are added to PBS buffer saline nanoparticle suspension (250 μL). After mixing, a clear solution is obtained and this solution is sonicated for 2 minutes before measuring absorbance at 260 nm. The encapsulated nucleic acid concentration and loading efficiency is calculated according to equations (1) and (2):

Cₑₙ(μg/ml)=A₂₆₀×OD₂₆₀ unit(μg/mL)×dilution factor(μL/μL) (1)

where the dilution factor is given by the assay volume (μL) divided by the sample stock volume (μL).

Encapsulation efficiency (%)=[Cₑₙ/Cᵢₙᵢₜᵢₐₗ]×100 (2)

where Cₑₙ is the nucleic acid (i.e., LNA oligonucleotide) concentration encapsulated in nanoparticle suspension after purification, and Cᵢₙᵢₜᵢₐₗ is the initial nucleic acid (LNA oligonucleotide) concentration before the formation of the nanoparticle suspension. Examples of various nanoparticle compositions are summarized in Tables 5 and 6.

TABLE 5

| Sample | | | |
|---|---|---|---|
| No | Nanoparticle Composition | Molar Ratio | Oligo |
| 1 | Cationic lipid 1:epd 10:DSPC:Chol:PEG-DSPE | 15:15:20:40:10 | Oligo-1 |
| 2 | Cationic lipid 1:cpd 10:DSPC:Chol:PEG-DSPE | 15:5:20:50:10 | Oligo-1 |
| 3 | Cationic lipid 1:cpd 10:DSPC:Chol:PEG-DSPE | 25:15:20:30:10 | Oligo-1 |
| 4 | Cationic lipid 1:cpd 10:Chol:PEG-DSPE | 20:47:30:3 | Oligo-1 |
| 5 | Cationic lipid 1:cpd 10:Chol:PEG-DSPE | 17:60:20:3 | Oligo-1 |
| 6 | Cationic lipid 1:cpd 10:PEG-DSPE | 20:78:2 | Oligo-1 |
| 7 | Cationic lipid 1:cpd 10:Chol:C16mPEG-Ceramide | 17:60:20:3 | Oligo-2 |
| 8 | Cationic lipid 1:cpd 10:Chol:PEG-DSPE:C16mPEG- | 18:60:20:1:1 | Oligo-2 |
| | Ceramide | | |

TABLE 6

| Sample | Nanoparticle | | |
|---|---|---|---|
| No. | Composition | Molar Ratio | Oligo |
| NP1 | Cationic lipid 1:cpd 10:Chol: | 18:60:20:1:1 | Oligo-2 |
| | PEG-DSPE:C16mPEG-Ceramide | | |
| NP2 | Cationic lipid 1:cpd 10:Chol: | 18:60:20:1:1 | FAM-Oligo- |
| | PEG-DSPE:C16mPEG-Ceramide | | 2 |
| NP3 | Cationic lipid 1:cpd 10:Chol: | 18:60:20:1:1 | None |
| | PEG-DSPE:C16mPEG-Ceramide | | |

### Example 17

### Nanoparticle Stability

Nanoparticle stability is defined as their capability to retain the structural integrity in PBS buffer at 4° C. over time. The colloidal stability of nanoparticles is evaluated by monitoring changes in the mean diameter over time. Nanoparticles prepared by Sample No. NP1 in Table 6 are dispersed in 10 mM PBS buffer (138 mM NaCl, 2.7 mM KCl, pH 7.4) and stored at 4° C. At a given time point, about 20-50 μL of the nanoparticle suspension is taken and diluted with pure water up to 2 mL. The sizes of nanoparticles are measured by DLS at 25° C.

### Example 18

### In Vitro Nanoparticle Cellular Uptake

The efficiency of cellular uptake of nucleic acids (LNA oligonucleotide Oligo-2) encapsulated in the nanoparticle described herein is evaluated in human cancer cells such as prostate cancer cells (15PC3 cell line). Nanoparticles of Sample NP2 are prepared using the method described in Example 16. LNA oligonucleotides (Oligo-2) are labeled with FAM for fluorescent microscopy studies.

The nanoparticles are evaluated in the 15PC3 cell line. The cells are maintained in a complete medium (DMEM, supplemented with 10% FBS), A 12 well plate containing 2.5×10⁵ cells in each well is incubated overnight at 37° C. The cells are washed once with Opti-MEM and 400 mL of Opti-MEM is added to each well. Then, the cells are treated with a nanoparticle solution of Sample No. NP2 (200 nM) encapsulating nucleic acids (FAM-modified Oligo 2) or a solution of free nucleic acids without the nanoparticles (naked FAM-modified Oligo 2) as a control. The cells are incubated for 24 hours at 37° C. The cells are washed with PBS five times, and then stained with 300 mL of Hoechst solution (2 mg/mL) per well for 30 minutes, followed by washing with PBS 5 times. The cells are fixed with pre-cooled (−20° C.) 70% EtOH at −20° C. for 20 minutes. The cells are inspected under fluorescent microscope to evaluate the efficiency of cellular uptake of nucleic acids encapsulated within the nanoparticle described herein.

### Example 19

### In Vitro Efficacy of Nanoparticles on mRNA Down-Regulation in a Variety of Human Cancer Cells

The efficacy of the nanoparticles described herein is evaluated in a variety of cancer cells, for example, human epideram cancer cells (A431), human gastric cancer cells (N87), human lung cancer cells (A549, HCC827, or H1581), human prostate cancer cells (15PC3, LNCaP, PC3, CWR22, DU145), human breast cancer cells (MCF7, SKBR3), colon cancer cells (SW480), pancreatic cancer cells (BxPC3), and melanoma (518A2). The cells are treated with one of the following: nanoparticles encapsulating antisense ErbB3 oligonucleotides (Sample NP1), or empty placebo nanoparticles (Sample No. NP3). The in vitro efficacy of each of the nanoparticles on downregulation of ErbB3 expression is measured by the procedures described in Example 3.

### Example 20

### Effects of Nanoparticles on mRNA Down-Regulation in Tumor and Liver of Human Prostate Cancer Xenografted Mice Model

The in vivo efficacy of nanoparticles described herein is evaluated in human prostate cancer xenografted mice. The 15PC3 human prostate tumors are established in nude mice by subcutaneous injection of 5×10⁶ cells/mouse into the right auxiliary flank. When tumors reach the average volume of 100 mm³, the mice are randomly grouped 5 mice per group. The mice of each group are treated with nanoparticle encapsulating antisense ErbB3 oligonucleotides (Sample NP1) or corresponding naked oligonucleotides (Oligo 2). The nanoparticles are given intravenously (i.v.) at 15 mg/kg/dose, 5 mg/kg/dose, 1 mg/kg/dose, or 0.5 mg/kg/dose at q3d×4 (or q3d×10). The dosage amount is based on the amount of oligonucleotides in the nanoparticles. The naked oligonucleotides are given intraperitoneally (i.p.) at 30 mg/kg/dose or intravenously at 25 mg/kg/dose or 45 mg/kg/dose at q3d×4 for 12 days. The mice are sacrificed twenty four hours after the final dose. Plasma samples are collected from the mice and stored at −20° C. Tumor and liver samples are also collected from the mice. The samples are analyzed for mRNA KD in the tumors and livers. The survival of the animals is observed.

1. A compound of Formula (I):
R-(L₁)ₐ-M-(L₂)_{b}-Q (I)
wherein
R is a water soluble neutral charged or zwitterion-containing moiety;
L₁₋₂ are independently selected bifunctional linkers;
M is an imine-containing moiety;
Q is a substituted or unsubstituted, saturated or unsaturated C4-30-containing moiety;
(a) is 0 or a positive integer; and
(b) is 0 or a positive integer.

2. The compound of claim 1, wherein M is —N═CR₁— or —CR₁═N—, wherein R₁ is hydrogen, C₁₋₆ alkyl, C₃₋₈ branched alkyl, C₃₋₈ cycloalkyl, C₁₋₆ substituted alkyl, C₃₋₈ substituted cycloalkyl, aryl and substituted aryl.

3. The compound of claim 1, wherein the zwitterion-containing moiety includes an amine and an acid, wherein the acidic proton is positioned three to eight atoms from the amine, and the acid is a carboxylic acid, a sulfonic acid, or a phosphoric acid.

4. (canceled)

5. The compound of claim 3, wherein the zwitterion-containing moiety is a zwitterionic form of an amino acid.

6. The compound of claim 1, wherein Q has Formula (Ia): 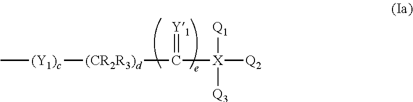
wherein
Y₁ and Y′₁ are independently O, S or NR₄;
(c) is 0 or 1;
(d) is 0 or a positive integer;
(e) is 0 or 1;
X is C, N or P;
Q₁ is H, C₁₋₃ alkyl, NR₅, OH, or 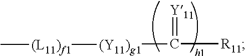
Q₂ is H, C₁₋₃ alkyl, NR₆, OH, or 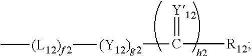
Q₃ is a lone electron pair, (═O), H, C₁₋₃ alkyl, NR₇, OH, or 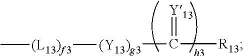
provided that
(i) when X is C, Q₃ is not a lone electron pair or (═O);
(ii) when X is N, Q₃ is a lone electron pair; and
(iii) when X is P, Q₃ is (═O) and (e) is zero,
wherein
L₁₁, L₁₂ and L₁₃ are independently selected bifunctional spacers;
Y₁₁, Y′₁₁, Y₁₂, Y′₁₂, Y₁₃, and Y′₁₃ are independently O, S or NR₈;
R₁₁, R₁₂ and R₁₃ are independently substituted or unsubstituted, saturated or unsaturated C₄₋₃₀;
(f1), (f2) and (f3) are independently 0 or 1;
(g1), (g2) and (g3) are independently 0 or 1; and
(h1), (h2) and (h3) are independently 0 or 1;
R₂₋₃ are independently selected from the group consisting of hydrogen, hydroxyl, amine, substituted amine, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, heteroalkyl, and substituted C₁₋₆ heteroalkyl; and
R₄₋₈ are independently selected from the group consisting of hydrogen, C₁₋₆alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, and substituted C₁₋₆heteroalkyl,
provided that Q includes at least one or two of R₁₁, R₁₂ and R₁₃.

7. The compound of claim 6, having Formula (Ib) or (I′b):

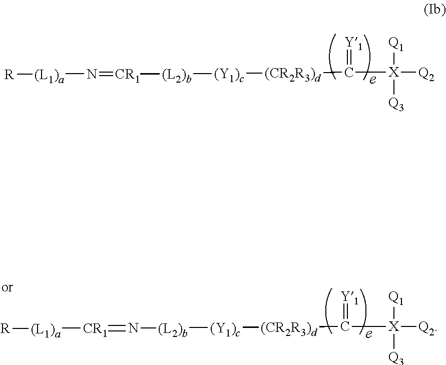

8. The compound of claim 6, wherein Q₁₋₃ independently include groups selected from C12-22 alkyl, C12-22 alkenyl, C12-22 alkyloxy, auroyl (C12), myristoyl (C14), palmitoyl (C16), stearoyl (C18), oleoyl (C18), and erucoyl (C22); saturated or unsaturated C12 alkyloxy, C14 alkyloxy, C16 alkyloxy, C18 alkyloxy, C20 alkyloxy, and C22 alkyloxy; and saturated or unsaturated C12 alkyl, C14 alkyl, C16 alkyl, C18 alkyl, C20 alkyl and C22 alkyl.

9. The compound of claim 6, wherein L₁₁, L₁₂ and L₁₃ are independently selected from the group consisting of:
—(CR₃₁R₃₂)_{q1}—,
—Y₂₆(CR₃₁R₃₂)_{q1}—,
—CH₂—, —(CH₂)₂—, —(CH₂)₃—, —(CH₂)₄—, —(CH₂)₅—, —(CH₂)₆—, —O(CH₂)₂—, —O(CH₂)₃—, —O(CH₂)₄—, —O(CH₂)₅—, —O(CH₂)₆—, and CH(OH)—,
wherein:
Y₂₆ is O, NR₃₃, or S;
R₃₁₋₃₂ are independently selected from the group consisting of hydrogen, hydroxyl, C₁₋₆ alkyls, C₃₋₁₂ branched alkyls, C₃₋₈ cycloalkyls, C₁₋₆ substituted alkyls, C₃₋₈ substituted cycloalkyls, C₁₋₆ heteroalkyls, substituted C₁₋₆ heteroalkyls, C₁₋₆ alkoxy, phenoxy and C₁₋₆ heteroalkoxy;
R₃₃ is selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, C₁₋₆ heteroalkyls, substituted C₁₋₆ heteroalkyls, C₁₋₆ alkoxy, phenoxy and C₁₋₆ heteroalkoxy; and
(q1) is zero or a positive integer.

10. (canceled)

11. The compound of claim 1, wherein L₁ is selected from the group consisting of:
—(CR₂₁R₂₂)ₜ₁—[C(═Y₁₆)]ₐ₃—,
—(CR₂₁R₂₂)ₜ₁Y₁₇—(CR₂₃R₂₄)ₜ₂—(Y₁₈)ₐ₂—[C(═Y₁₆)]ₐ₃—,
—(CR₂₁R₂₂CR₂₃R₂₄Y₁₇)ₜ₁—[C(═Y₁₆)]ₐ₃—,
—(CR₂₁R₂₂CR₂₃R₂₄Y₁₇)ₜ₁(CR₂₅R₂₆)ₜ₄—(Y₁₈)ₐ₂—[C(═Y₁₆)]ₐ₃—,
—[(CR₂₁R₂₂CR₂₃R₂₄)ₜ₂Y₁₇]ₜ₃(CR₂₅R₂₆)ₜ₄—(Y₁₈)ₐ₂—[C(═Y₁₆)]ₐ₃—,
—(CR₂₁R₂₂)ₜ₁—[(CR₂₃R₂₄)ₜ₂Y₁₇]ₜ₃(CR₂₅R₂₆)ₜ₄≦(Y₁₈)ₐ₂—[C(═Y₁₆)]ₐ₃—,
—(CR₂₁R₂₂)ₜ₁(Y₁₇)ₐ₂[C(═Y₁₆)]ₐ₃(CR₂₃R₂₄)ₜ₂—,
—(CR₂₁R₂₂)ₜ₁(Y₁₇)ₐ₂[C(═Y₁₆)]ₐ₃Y₁₄(CR₂₃R₂₄)ₜ₂—,
—(CR₂₁R₂₂)ₜ₁(Y₁₇)ₐ₂[C(═Y₁₆)]ₐ₃(CR₂₃R₂₄)ₜ₂—Y₁₅—(CR23R₂₄)ₜ₃—,
—(CR₂₁R₂₂)ₜ₁(Y₁₇)ₐ₂[C(═Y₁₆)]ₐ₃Y₁₄(CR₂₃R₂₄)ₜ₂—Y₁₅—(CR₂₃R₂₄)ₜ₃—,
—(CR₂₁R₂₂)ₜ₁(Y₁₇)ₐ₂[C(═Y₁₆)]ₐ₃(CR₂₃R₂₄CR₂₅R₂₆Y₁₉)ₜ₂(CR₂₇CR₂₈)ₜ₃—,
—(CR₂₁R₂₂)ₜ₁(Y₁₇)ₐ₂[C(═Y₁₆)]ₐ₃Y₁₄(CR₂₃R₂₄CR₂₅R₂₆Y₁₉)ₜ₂(CR₂₇CR₂₈)ₜ₃—, 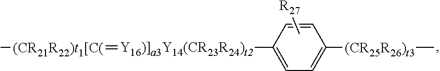
—CH₂—, —(CH₂)₂—, —(CH₂)₃—, —(CH₂)₄—, —(CH₂)₅—, —(CH₂)₆—, —NH(CH₂)—,
—CH(NH₂)CH₂—,
—(CH₂)₄—C(═O)—, —(CH₂)₅—C(═O)—, —(CH₂)₆—C(═O)—,
—CH₂CH₂O—CH₂O—C(═O)—,
—(CH₂CH₂O)₂—CH₂O—C(═O)—,
—(CH₂CH₂O)₃—CH₂O—C(═O)—,
—(CH₂CH₂O)₂—C(═O)—,
—CH₂CH₂O—CH₂CH₂NH—C(═O)—,
—(CH₂CH₂O)₂—CH₂CH₂NH—C(═O)—,
—CH₂—O—CH₂CH₂O—CH₂CH₂NH—C(═O)—,
—CH₂—O—(CH₂CH₂O)₂—CH₂CH₂NH—C(═O)—,
—CH₂—O—CH₂CH₂O—CH₂C(═O)—,
—CH₂—O—(CH₂CH₂O)₂—CH₂C(═O)—,
—(CH₂)₄—C(═O)NH—, —(CH₂)₅—C(═O)NH—,
—(CH₂)₆—C(═O)NH—,
—CH₂CH₂O—CH₂O—C(═O)—NH—,
—(CH₂CH₂O)₂—CH₂O—C(═O)—NH—,
—(CH₂CH₂O)₃—CH2O—C(═O)—NH—,
—(CH₂CH₂O)₂—C(═O)—NH—,
—CH₂CH₂O—CH₂CH₂NH—C(═O)—NH—,
—(CH₂CH₂O)₂—CH₂CH₂NH—C(═O)—NH—,
—CH₂—O—CH₂CH₂O—CH₂CH₂NH—C(═O)—NH—,
—CH₂—O—(CH₂CH₂O)₂—CH₂CH₂NH—C(═O)—NH—,
—CH₂—O—CH₂CH₂O—CH₂C(═O)—NH—,
—CH₂—O—(CH₂CH₂O)₂—CH₂C(═O)—NH—,
—(CH₂CH₂O)₂—, —CH₂CH₂O—CH₂O—,
—(CH₂CH₂O)₂—CH₂CH₂NH—,
—(CH₂CH₂O)₃—CH₂CH₂NH—,
—CH₂CH₂O—CH₂CH₂NH—,
—(CH₂CH₂O)₂—CH₂CH₂NH—,
—CH₂—O—CH₂CH₂O—CH₂CH₂NH—,
—CH₂—O—(CH₂CH₂O)₂—CH₂CH₂NH—,
—CH₂—O—CH₂CH₂O—,
—CH₂—O—(CH₂CH₂O)₂—, 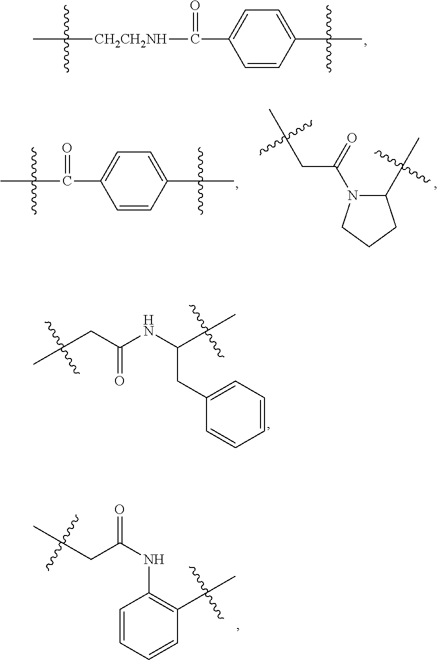
—C(═O)NH(CH₂)₂—, —CH₂C(═O)NH(CH₂)₂—,
—C(═O)NH(CH₂)₃—, —CH₂C(═O)NH(CH₂)₃—,
—C(═O)NH(CH₂)₄—, —CH₂C(═O)NH(CH₂)₄—,
—C(═O)NH(CH₂)₅—, —CH₂C(═O)NH(CH₂)₅—,
—C(═O)NH(CH₂)₆—, —CH₂C(═O)NH(CH₂)₆—,
—C(═O)O(CH₂)₂—, —CH₂C(═O)O(CH₂)₂—,
—C(═O)O(CH₂)₃—, —CH₂C(═O)O(CH₂)₃—,
—C(═O)O(CH₂)₄—, —CH₂C(═O)O(CH₂)₄—,
—C(═O)O(CH₂)₅—, —CH₂C(═O)O(CH₂)₅—,
—C(═O)O(CH₂)₆—, —CH₂C(═O)O(CH₂)₆—,
—(CH₂CH₂)₂NHC(═O)NH(CH₂)₂—,
—(CH₂CH₂)₂NHC(═O)NH(CH₂)₃—,
—(CH₂CH₂)₂NHC(═O)NH(CH₂)₄—,
—(CH₂CH₂)₂NHC(═O)NH(CH₂)₅—,
—(CH₂CH₂)₂NHC(═O)NH(CH₂)₆—,
—(CH₂CH₂)₂NHC(═O)O(CH₂)₂—,
—(CH₂CH₂)₂NHC(═O)O(CH₂)₃—,
—(CH₂CH₂)₂NHC(═O)O(CH₂)₄—,
—(CH₂CH₂)₂NHC(═O)O(CH₂)₅—,
—(CH₂CH₂)₂NHC(═O)O(CH₂)₆—,
—(CH₂CH₂)₂NHC(═O)(CH₂)₂—,
—(CH₂CH₂)₂NHC(═O)(CH₂)₃—,
—(CH₂CH₂)₂NHC(═O)(CH₂)₄—,
—(CH₂CH₂)₂NHC(═O)(CH₂)₅—, and
—(CH₂CH₂)₂NHC(═O)(CH₂)₆—,
wherein:
Y₁₆ is O, NR₂₈, or S;
Y₁₄₋₁₅ and Y₁₇₋₁₉ are independently O, NR₂₉, or S;
R₂₁₋₂₇ are independently selected from the group consisting of hydrogen, hydroxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy; and
R₂₈₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t1), (t2), (t3) and (t4) are independently zero or positive integers; and
(a2) and (a3) are independently zero or 1.

12. (canceled)

13. The compound of claim 1, wherein L₂ is selected from the group consisting of:
—(CR′₂₁R′₂₂)_{t′1}—[C(═Y′₁₆)]_{a′3}(CR′₂₇CR′₂₈)_{t′2}—,
—(CR′₂₁R′₂₂)_{t′1}Y′₁₄—(CR′₂₃R′₂₄)_{t′2}—(Y′₁₅)_{a′2}—[C(═Y′₁₆)]_{n′3}(CR′₂₇CR′₂₈)_{t′3}—,
—(CR′₂₁R′₂₂CR′₂₃R′₂₄Y′₁₄)ₜ₊₁—[C(═Y′₁₆)]_{a′3}(CR′₂₇CR′₂₈)_{t′2}—,
—(CR′₂₁R′₂₂CR′₂₃R′₂₄Y′₁₄)_{t′1}(CR′₂₅R′₂₆)_{t′2}—(Y′₁₅)_{a′2}—[C(═Y′₁₆)]_{a′3}(CR′₂₇CR′₂₈)_{t′3}—,
—[(CR′₂₁R′₂₂CR′₂₃R′₂₄)_{t′2}Y′₁₄]_{t′1}(CR′₂₅R′₂₆)_{t′2}—(Y′₁₅)_{a′2}—[C(═Y′₁₆)]_{a′3}(CR′₂₇CR′₂₈)_{t′3}—,
—(CR′₂₁R′₂₂)_{t′1}—[(CR′₂₃R′₂₄)_{t′2}Y′₁₄]_{t′2}(CR′₂₅R′₂₆)_{t′3}—(Y′₁₅)_{a′2}—[C(═Y′₁₆)]_{a′3}(CR′₂₇CR′₂₈)_{t′4}—
—(CR′₂₁R′₂₂)_{t′1}(Y′₁₄)_{a′2}[C(═Y′₁₆)]_{a′3}(CR′₂₃R′₂₄)_{t′2}—,
—(CR′₂₁R′₂₂)_{t′1}(Y′₁₄)_{a′2}[C(═Y′₁₆)]_{a′3}(CR′₂₃R′₂₄)_{t′2}—,
—(CR′₂₁R′₂₂)_{t′1}(Y′₁₄)_{a′2}[C(═Y′₁₆)]_{a′3}(CR′₂₃R′₂₄)_{t′2}—Y′₁₅—(CR′₂₃R′₂₄)_{t′3}—,
—(CR′₂₁R′₂₂)_{t′1}(Y′₁₄)_{a′2}[C(═Y′₁₆)]_{a′3}(CR′₂₃R′₂₄)_{t′2}—Y′₁₅—(CR′₂₃R′₂₄)_{t′3}—,
—(CR′₂₁R′₂₂)_{t′1}(Y′₁₄)_{a′2}[C(═Y′₁₆)]_{a′3}(CR′₂₃R′₂₄CR′₂₅R′₂₆Y′₁₅)_{t′2}(CR′₂₇CR′₂₈)_{t′3}—,
—(CR′₂₁R′₂₂)_{t′1}(Y′₁₄)_{a′2}[C(═Y′₁₆)]_{a′3}(CR′₂₃R′₂₄CR′₂₅R′₂₆Y′₁₅)_{t′2}(CR′₂₇CR′₂₈)_{t′3}—, 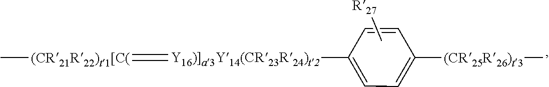
—CH₂—, —(CH₂)₂—, —(CH₂)₃—,—(CH₂)₄—, —(CH₂)₅—, —(CH₂)₆—, —NH(CH₂)—,
—CH(NH₂)CH₂—,
—O(CH₂)₂—, —C(═O)O(CH₂)₃—, —C(═O)NH(CH₂)₃—,
—C(═O)O(CH₂)₂, —C(═O)(CH₂)₃—,
—CH₂—C(═O)—O(CH₂)₃—,
—CH₂—C(═O)—NH(CH₂)₃—,
—CH₂—OC(═O)—O(CH₂)₃—,
—CH₂—OC(═O)—NH(CH₂)₃—,
—(CH₂)₂—C(═O)—O(CH₂)₃—,
—(CH₂)₂)—C(═O)—NH(CH₂)₃—,
—CH₂C(═O)O(CH₂)₂—O—(CH₂)₂—,
—CH₂C(═O)NH(CH₂)₂—O—(CH₂)₂—,
—(CH₂)₂C(═O)O(CH₂)₂—O—(CH₂)₂—,
—(CH₂)₂C(═O)NH(CH₂)₂—O—(CH₂)₂—,
—CH₂C(═O)O(CH₂CH₂O)₂CH₂CH₂—,
—(CH₂)₂C(═O)O(CH₂CH₂O)₂CH₂CH₂—,
—(CH₂CH₂O)₂—, —CH₂CH₂O—CH₂O—,
—(CH₂CH₂O)₂—CH₂CH₂NH—, —(CH₂CH₂O)₃—CH₂CH₂NH—,
—CH₂CH₂O—CH₂CH₂NH—,
—CH₂—O—CH₂CH₂O—CH₂CH₂NH—,
—CH₂—O—(CH₂CH₂O)₂—CH₂CH₂NH—,
—CH₂—O—CH₂CH₂O—, —CH₂—O—(CH₂CH₂O)₂—, 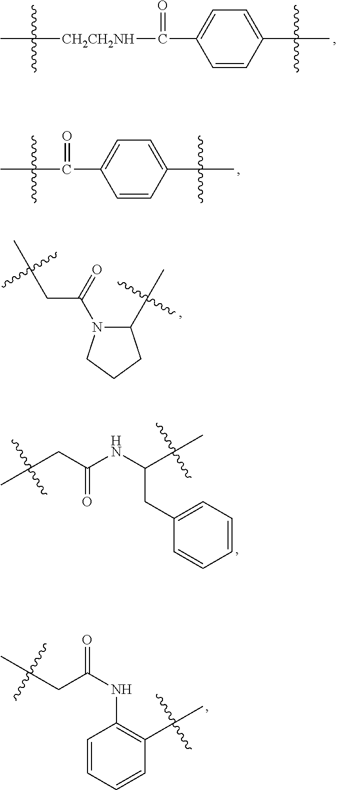
—(CH₂)₂NHC(═O)—(CH₂CH₂O)₂—,
—C(═O)NH(CH₂)₂—, —CH₂C(═O)NH(CH₂)₂—,
—C(═O)NH(CH₂)₃—, —CH₂C(═O)NH(CH₂)₃—,
—C(═O)NH(CH₂)₄—, —CH₂C(═O)NH(CH₂)₄—,
—C(═O)NH(CH₂)₅—, —CH₂C(═O)NH(CH₂)₅—,
—C(═O)NH(CH₂)₆—, —CH₂C(═O)NH(CH₂)₆—,
—C(═O)O(CH₂)₂—, —CH₂C(═O)O(CH₂)₂—,
—C(═O)O(CH₂)₃—, —CH₂C(═O)O(CH₂)₃—,
—C(═O)O(CH₂)₄—, —CH₂C(═O)O(CH₂)₄—,
—C(═O)O(CH₂)₅—, —CH₂C(═O)O(CH₂)₅—,
—C(═O)O(CH₂)₆—, —CH₂C(═O)O(CH₂)₆—,
—(CH₂CH₂)₂NHC(═O)NH(CH₂)₂—,
—(CH₂CH₂)₂NHC(═O)NH(CH₂)₃—,
—(CH₂CH₂)₂NHC(═O)NH(CH₂)₄—,
—(CH₂CH₂)₂NHC(═O)NH(CH₂)₅—,
—(CH₂CH₂)₂NHC(═O)NH(CH₂)₆—,
—(CH₂CH₂)₂NHC(═O)O(CH₂)₂—,
—(CH₂CH₂)₂NHC(═O)O(CH₂)₃—,
—(CH₂CH₂)₂NHC(═O)O(CH₂)₄—,
—(CH₂CH₂)₂NHC(═O)O(CH₂)₅—,
—(CH₂CH₂)₂NHC(═O)O(CH₂)₆—,
—(CH₂CH₂)₂NHC(═O)(CH₂)₂—,
—(CH₂CH₂)₂NHC(═O)(CH₂)₃—,
—(CH₂CH₂)₂NHC(═O)(CH₂)₄—,
—(CH₂CH₂)₂NHC(═O)(CH₂)₅—, and
—(CH₂CH₂)₂NHC(═O)(CH₂)₆—,
wherein:
Y′₁₆ is O, NR′₂₈, or S;
Y′₁₄₋₁₅ and Y′₁₇ are independently O, NR′₂₉, or S;
R′₂₁₋₂₇ are independently selected from the group consisting of hydrogen, hydroxyl, amine, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
R′₂₈₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t′1), (t′2), (t′3) and (t′4) are independently zero or positive integers; and
(a′2) and (a′3) are independently zero or 1.

14. (canceled)

15. The compound of claim 1, wherein Q is selected from the group consisting of: 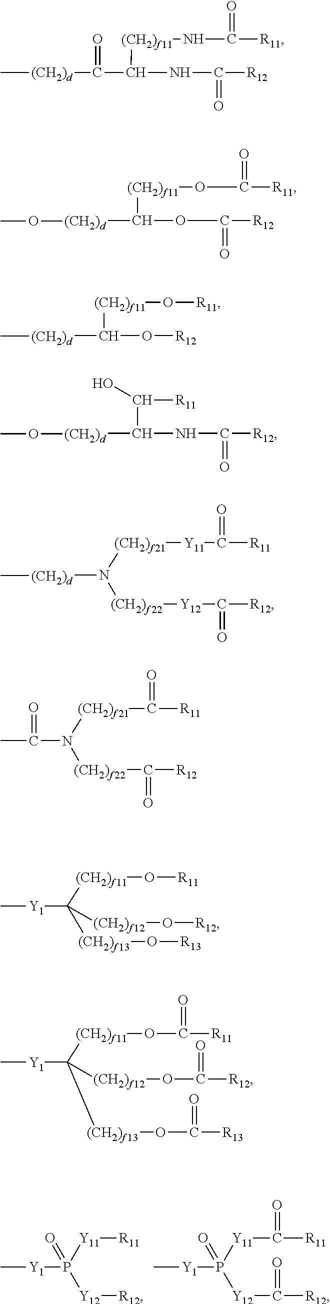 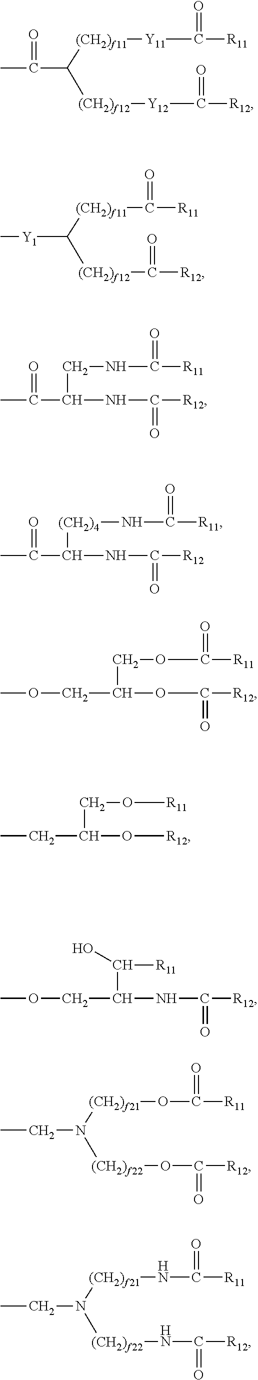 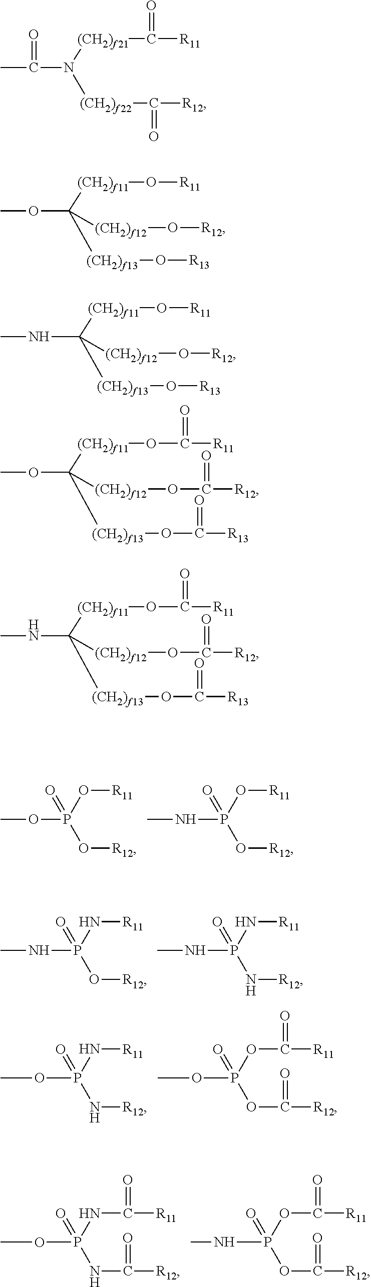 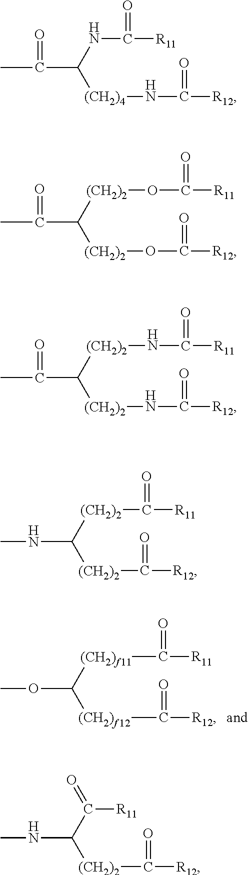
wherein
Y₁ is O, S, or NR₃₁;
R₁₁, R₁₂, and R₁₃ are independently selected from the group consisting of substituted or unsubstituted, saturated or unsaturated C₄₋₃₀, the same or different C12-22 saturated or unsaturated aliphatic hydrocarbons;
R₃₁ is hydrogen, methyl or ethyl;
(d) is 0 or a positive integer; and
(f11), (f12) and (f13) are independently 0, 1, 2, 3, or 4; and
(f21) and (f22) are independently 1, 2, 3 or 4.

16. (canceled)

17. The compound of claim 1 selected from the group consisting of:

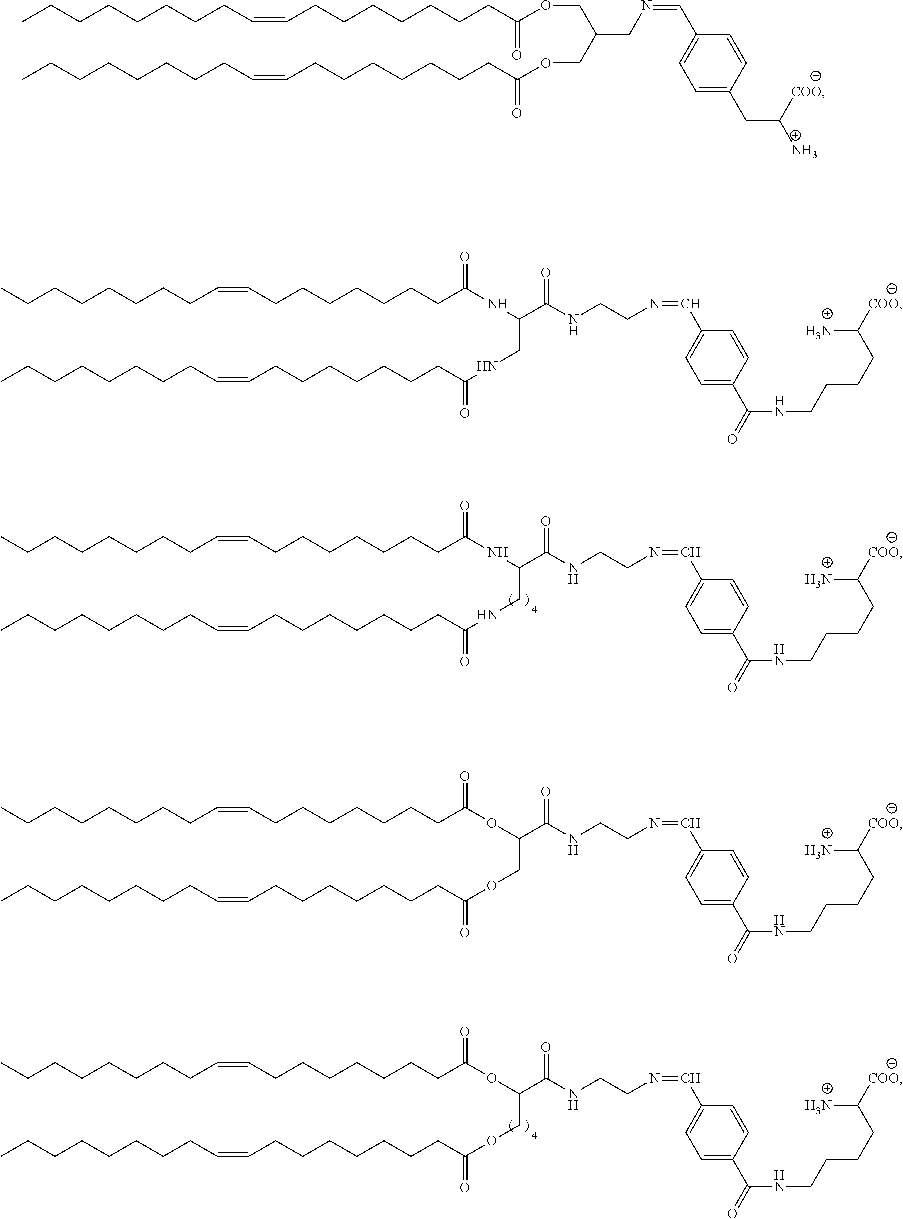

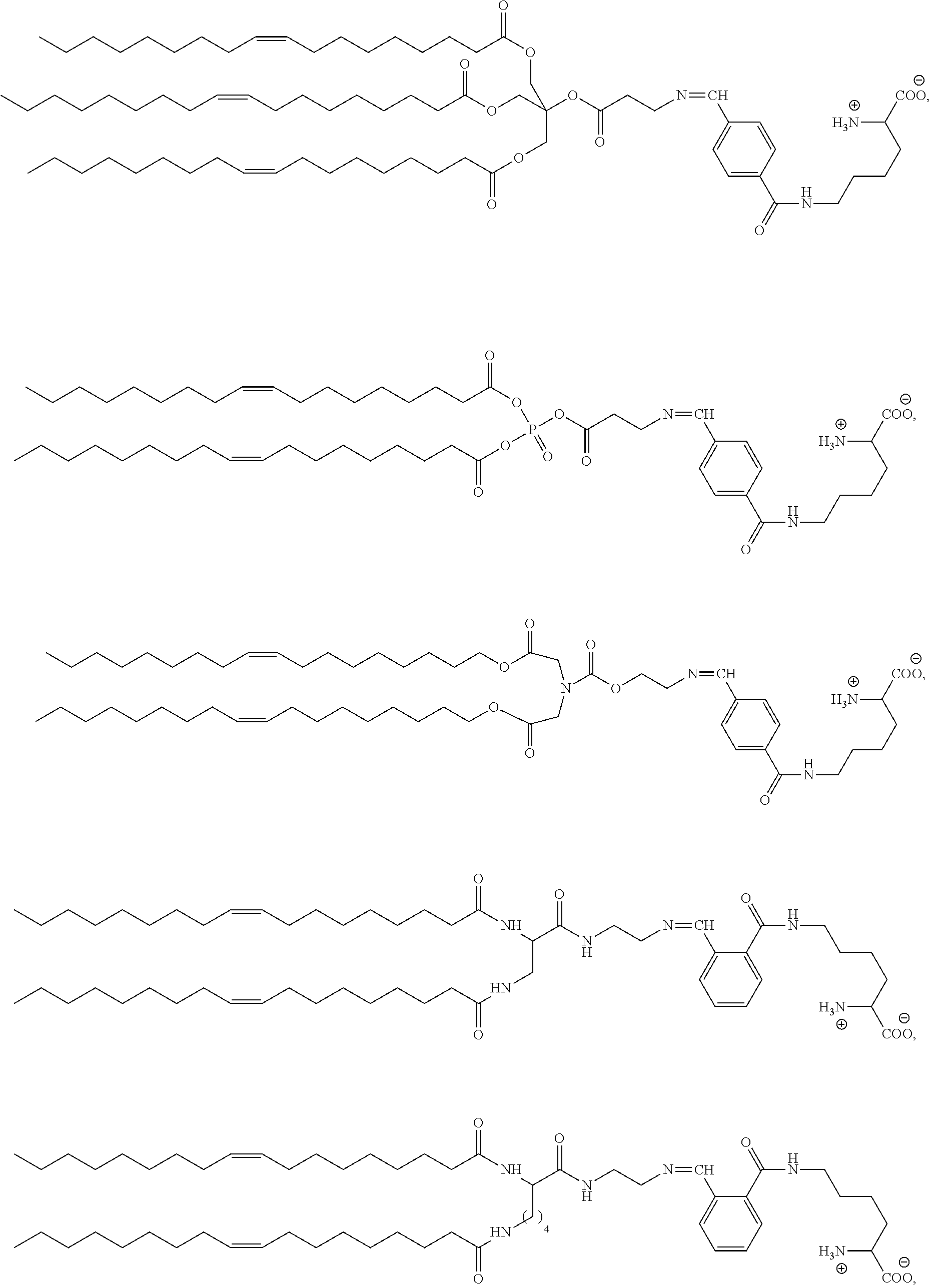

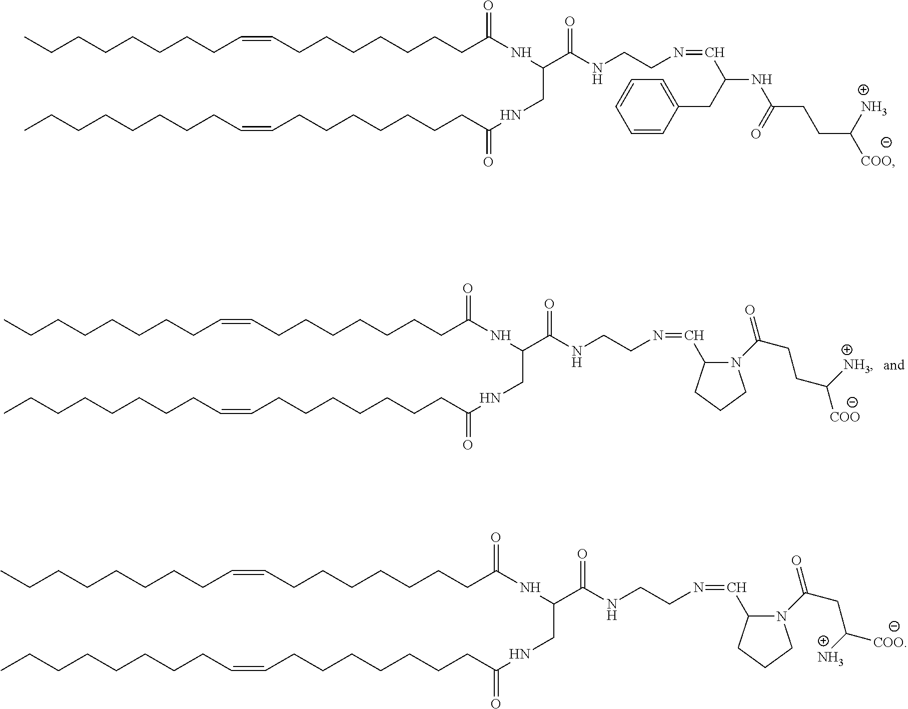

18. A nanoparticle composition comprising a compound of Formula (I) of claim 1.

19. The nanoparticle composition of claim 18, wherein the compound of Formula (I) is

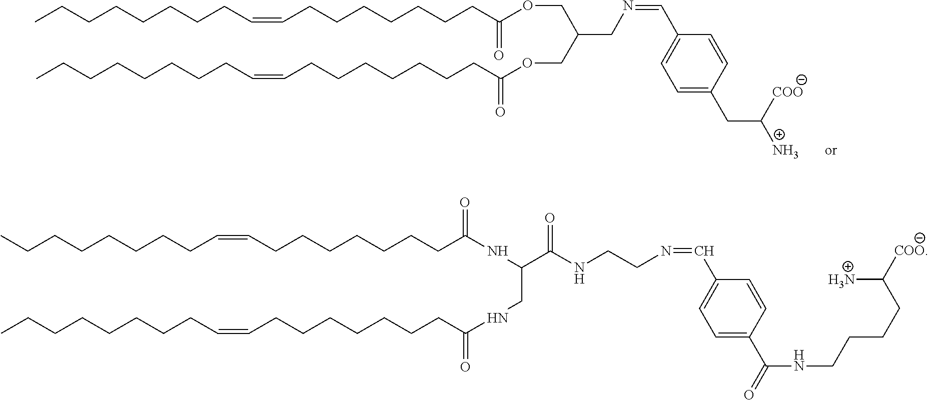

20. The nanoparticle composition of claim 18, further comprising a cationic lipid, and a PEG-lipid.

21. The nanoparticle composition of claim 20, wherein the cationic lipid is

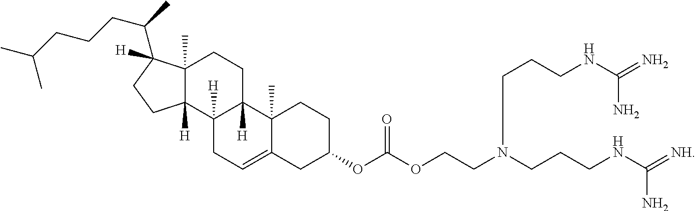

22. The nanoparticle composition of claim 20, wherein the PEG lipid is selected from the group consisting of PEG-DSPE, PEG-dipalmitoylglycamide, C16mPEG-ceramide and combinations thereof.

23. The nanoparticle composition of claim 20, further comprising cholesterol.

24. The nanoparticle composition of claim 20, wherein the cationic lipid has a molar ratio ranging from about 10% to about 99.9% of the total lipid present in the nanoparticle composition.

25. (canceled)

26. The nanoparticle composition of claim 24, wherein a molar ratio of a cationic lipid, a fusogenic lipid including a compound of Formula (I), a PEG-lipid, and cholesterol is about 15-25%:20-78%:0-50%:2-10%: of the total lipid present in the nanoparticle composition.

27. The nanoparticle composition of claim 18 comprising nucleic acids encapsulated with the nanoparticle composition.

28. The nanoparticle of claim 27, wherein the nucleic acids is a single stranded or double stranded oligonucleotide.

29. The nanoparticle of claim 27, wherein the nucleic acids are selected from the group consisting of deoxynucleotide, ribonucleotide, locked nucleic acids (LNA), short interfering RNA (siRNA), microRNA (miRNA), aptamers, peptide nucleic acid (PNA), phosphorodiamidate morpholino oligonucleotides (PMO), tricyclo-DNA, double stranded oligonucleotide (decoy ODN), catalytic RNA (RNAi), aptamers, spiegelmers, CpG oligomers and combinations thereof.

30.-33. (canceled)

34. The nanoparticle of claim 28, wherein the oligonucleotide inhibits expression of oncogenes, pro-angiogenesis pathway genes, pro-cell proliferation pathway genes, viral infectious agent genes, and pro-inflammatory pathway genes.

35. The nanoparticle of claim 28, wherein the oligonucleotide is selected from the group consisting of antisense bcl-2 oligonucleotides, antisense HIF-1α oligonucleotides, antisense survivin oligonucleotides, antisense ErbB3 oligonucleotides, antisense PIK3CA oligonucleotides, antisense HSP27 oligonucleotides, antisense androgen receptor oligonucleotides, antisense Gli2 oligonucleotides, and antisense beta-catenin oligonucleotides.

36. The nanoparticle of claim 28, wherein the oligonucleotide comprises eight or more consecutive nucleotides set forth in SEQ ID NO: 1, SEQ ID NOs 2 and 3, SEQ ID NO:4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16, and each nucleic acid is a naturally occurring or modified nucleic acid.

37. The nanoparticle of claim 27, wherein the charge ratio of the nucleic acids and the compound of Formula (I) ranges from about 1:20 to about 20:1.

38. The nanoparticle of claim 27, wherein the nanoparticle has a size ranging from about 50 nm to about 150 nm.

39.-40. (canceled)

41. A method of inhibiting or downregulating a gene expression in human cells or tissues, comprising:
contacting human cells or tissues with a nanoparticle of claim 27.

42. The method of claim 41, wherein the cells or tissues are cancer cells or tissues.

43. (canceled)

44. A method of inhibiting the growth or proliferation of cancer cells comprising:
contacting a cancer cell with a nanoparticle of claim 27.

45. The method of claim 44, further comprising administering an anticancer agent.
